(19) United States
(12) Patent Application Publication
(10) Pub. No.: US 2023/0295738 A1
(43) Pub. Date: Sep. 21, 2023

(54) SYSTEMS AND METHODS FOR DETECTION OF RESIDUAL DISEASE

(71) Applicants: CORNELL UNIVERSITY, Ithaca, NY (US); NEW YORK GENOME CENTER, New York, NY (US); THE BROAD INSTITUTE, INC., Cambridge, MA (US)

(72) Inventors: Dan Avi LANDAU, Brooklyn, NY (US); Asaf Ari ZVIRAN, Cresskill, NJ (US); Viktor A. ADALSTEINSSON, Wakefield, MA (US)

(21) Appl. No.: 18/133,524

(22) Filed: Apr. 12, 2023

Related U.S. Application Data
(63) Continuation of application No. 16/976,036, filed on Aug. 26, 2020.
(60) Provisional application No. 62/636,150, filed on Feb. 27, 2018.

(51) Int. Cl. C12Q 1/6886; G16B 20/00; G16B 40/20; G16B 30/00

(52) U.S. Cl. CPC C12Q 1/6886; G16B 20/00; G16B 40/20; G16B 30/00

(57) ABSTRACT

The disclosure relates to systems, software, and methods for the detection of residual disease, e.g., residual tumor disease, in subjects, e.g., human cancer patients.

### CROSS-REFERENCE TO RELATED APPLICATIONS

This application is a continuation of U.S. patent application Ser. No. 16/976,036, filed Aug. 20, 2020, which is a national phase application under 35 U.S.C. § 371 of International Application No. PCT/US2019/019907, filed Feb. 27, 2019, which claims priority to and the benefit of Provisional Application No. 62/636,150, filed Feb. 27, 2018. Both applications are hereby incorporated by reference in their entirety as if fully set forth below and for all applicable purposes.

### TECHNICAL FIELD

Embodiments of the disclosure generally relate to the field of medical diagnostics. In particular, embodiments of the disclosure relate to compositions, methods, and systems for tumor detection and diagnosis.

### INTRODUCTION

Cell-free circulating DNA (cfDNA) released from dying cells enables surveys of the somatic genome and epigenome dynamically over time for clinical purposes. The ability to obtain a biopsy through a simple blood draw allows for dynamic genomic measurement in a non-invasive manner. It can overcome spatial limitations, such as inaccessibility of lung tissue.

Circulating tumor DNA (ctDNA), not to be confused with cell-free DNA (cfDNA), can be found and measured in the blood of cancer patients. ctDNA has been shown to correlate with tumor burden and change in response to treatment or surgery (Diehl et al., Nature medicine, 14(9):985-990, 2008). ctDNA can be detected even in early stage non-small cell lung cancer (NSCLC) and therefore has the potential to transform NSCLC diagnosis and treatment (Sozzi et al., Journal of Clinical Oncology, 21(21), 3902-3908, 2003; Tie et al., Science translational medicine, 8(346):346ra92-346ra92, 2016; Bettegowda et al., Science translational medicine, 6(224): 224ra24-224ra24, 2014; Wang et al., Clinical Cancer Research, 16(4): 1324-1330, 2010).

One of the major areas of future promise for cfDNA-based cancer studies is in detection of residual disease (RD) to guide clinical interventions. For example, detection of residual disease after surgical resection can assist clinicians and patients to make decisions about costly and toxic adjuvant therapy. However, in the context of tumors with low burden, e.g., minimal residual disease (MRD), tumor fraction (TF) is significantly low. To enable mutation detection of low TF cfDNA, the prevailing paradigm has been to increase the depth of sequencing of a limited high yield target set (e.g., common cancer drivers or patient-specific panels that are sequenced to a depth of about 10,000 to 100,000 reads/base). Additionally, molecular and analytic approaches have been integrated with ultra-deep sequencing to reduce sequencing error and improve sensitivity of detection at low tumor fraction (TF).

While these state-of-the-art methods provide detection with high accuracy in some instances, they are hindered by a fundamental limitation that reduces detection sensitivity—limited input material. In MRD, the tumor burden is low, typical plasma samples contain only 1-10 ng/ml of cfDNA. The low amount of cfDNA translates into only hundreds to few thousands of genomic equivalents. Thus, the prevailing technique relying on ultra-deep sequencing (e.g., 100,000×) may be rendered ineffective by the limited number of physical fragments that cover each site that are present in the sample (e.g., 1000 genomic equivalents in 6 ng of cfDNA). Even with ultra-deep sequencing and advanced molecular error suppression, the limited input material imposes a detection limit on tumor fraction (TF) frequencies lower than 0.1-1%. As such, although detection of cancer with low tumor burden is clinically beneficial to patients and clinicians, existing methods relying on the identification of somatic mutations face significant challenges due to the low frequency of tumor-derived cfDNA sample.

Accordingly, there is an urgent but unmet need for minimally invasive systems and methods that allow detection of tumors, particularly, in the context of diagnosis of minimal residual disease (MRD) with limited input material. Effective diagnosis of tumors in the residual disease setting (e.g., after surgery and/or therapy) is advantageous from both economic as well as clinical standpoints. This is especially true in the context of lung cancer, as most patients are diagnosed with advanced stage disease with dismal outcome (Herbst et al., N Engl J Med., 359(13):1367-80, 2008).

### SUMMARY

The disclosure relates to methods and systems for diagnosing residual tumor disease by analyzing tumor-specific markers in a subject's sample (e.g., plasma sample or blood sample). The methods of the disclosure utilize algorithms and/or statistical classifiers to discriminate between quality markers and artefactual noise based on a number of parameters. For instance, wherein the marker is a single nucleotide variation (SNV), the algorithms of the disclosure classify such SNVs in the subject's genetic compendium as signal or noise on the basis of qualitative features of the markers such as, e.g., base-quality (BQ) of the SNV and mapping-quality (MQ) of the SNV. Similarly, wherein the marker is a copy number variation (CNV), the algorithms classify the CNVs in the compendium as signal or noise on the basis of parameters such as centromeric proximity, overlap with cfDNA coverage mask, and/or association of the CNV with low mappability (mapping quality; MQ) reads. Thus, from the subject's genetic compendium, markers that are likely to be associated with artefactual noise are eliminated and high quality markers are processed through robust, integrative mathematical model(s) that permit estimation of tumor fractions in the sample. If the estimated tumor fraction is found to be above a certain threshold, then a positive diagnosis can be made with high confidence. In contrast, if the estimated tumor fraction is below the threshold value, then a positive diagnosis is not made at that time.

In this context, simulated testing of plasma somatic mutation calling using synthetic mixtures of tumor and normal whole genome-sequencing data from lung patients with variable fraction of tumor reads ranging from 1% to 0.001% ( 1/100,000) reveals the strength and accuracy of the present methods over existing techniques.

The disclosure also relates to a plurality of indicators that are capable of suggesting that a variant detected via sequencing is not a true somatic mutation but rather an artifact of sequencing or mapping technology. In this context, previous studies have demonstrated that sequencing errors are not random and are likely related to both DNA sequence context and technical factors consequential of the sequencing technologies. The fidelity of sequencing is also limited by the length of each sequencing-read, with an increase in error rate as the read length increases. Errors may be imposed when reads are mapped to a reference genome. The mapping process is computationally intensive and complicated by the fact that the genome has variable regions, motifs, and repeatable elements. Short nucleotide reads may map to more than one location or not map at all. These limitations with the existing methodologies for sequencing/mapping of genomic data may be rectified using the systems and methods of the disclosure. The indicators of the disclosure are capable of calling true mutations from errors by analyzing a plurality of factors such as (i) low base quality; and/or (ii) low mapping quality, (iii) mutation position in read, and (iv) read fragment size in the case of SNV markers and (1) genomic position score, (2) cfDNA coverage mask (blacklist), (3) low mapping quality, (4) correlation between Log 2 and read group fragment size in the case of CNV markers.

The present systems and methods for detecting biomarkers associated with tumors are especially adapted to detection of low abundance markers. First, the model takes into account both quality metrics associated with the type of marker and the systems/methods used in the detection thereof, as well as subject-specific parameters, to compute an estimated tumor fraction (eTF). For instance, wherein the marker is SNV, the integrative mathematical model takes into account process quality metrics such as estimated coverage and noise and also subject-specific parameters such as mutation load. In the case of CNVs, the integrative mathematical model takes into account index factor, along with subject-specific features such as CNV directionality (e.g., amplifications are positively factored; deletions are negatively factored) to compute an estimated tumor fraction (eTF). Accordingly, the analytic approach of the present disclosure integrates genome-wide mutational information to allow sensitive analysis of samples containing cfDNA such that residual diseases can be diagnosed precisely and non-invasively.

The disclosure accordingly relates to the following non-limiting embodiments:

In various embodiments, a method for detecting residual disease in a subject in need thereof is provided. The method can comprise receiving a first subject-specific genome wide compendium of reads associated with genetic markers from a first biological sample of a subject. The first biological sample can comprise a baseline sample. The first compendium of reads can each comprise reads of a single base pair length (e.g., SNV or Indel) and wherein the baseline sample comprises a tumor sample or a plasma sample. The method can further comprise filtering artefactual sites from the first compendium of reads. The filtering can comprise removing, from the first compendium of genetic markers, recurring sites generated over a cohort of reference healthy samples. Alternatively, or in addition, the filtering can comprise identifying germ line mutations in peripheral blood mononuclear cells of the normal cell sample and removing said germ line mutations from the from the first compendium of genetic markers. The method can further comprise detecting reads from a second subject-specific genome wide compendium of genetic markers in a second biological sample of the subject to generate a tumor-associated genome-wide representation of genetic markers in the second sample. The method can further comprise filtering noise from the first and second genome-wide compendium of reads. The noise filtering can comprise using at least one error suppression protocol to produce a first filtered read set for the first genome-wide compendium of reads and a second filtered read set for the second genome-wide compendium of reads. The at least one error suppression protocol can comprise calculating the probability that any single nucleotide variation in the first and second compendium is an artefactual mutation, and removing said mutation. The probability can be calculated as a function of features selected from the group consisting of mapping-quality (MQ), variant base-quality (MBQ), position-in-read (PIR), mean read base quality (MRBQ), and combinations thereof. Alternatively, or in combination, the at least one error suppression protocol can include removing artefactual mutations using discordance testing between independent replicates of the same DNA fragment generated from polymerase chain reaction or sequencing processing. In addition to or alternative to the discordance testing, duplication consensus can be included, wherein artefactual mutations are identified and removed when lacking concordance across a majority of a given duplication family. The method can further comprise computing an estimated tumor fraction (eTF) of the first and second biological sample using the first and second filtered read sets by applying a background noise model to one or more integrative mathematical models. The method can further include detecting a residual disease in the subject if the estimated tumor fraction in the second biological sample exceeds an empirical threshold.

In various embodiments, a method for detecting residual disease in a subject in need thereof is provided. The method can comprise receiving a first subject-specific genome wide compendium of reads associated with genetic markers from a first biological sample of a subject. The biological sample can comprise a baseline sample. The first compendium of reads can each comprise a copy number variation (CNV) and wherein the baseline sample comprises a tumor sample or a plasma sample. The method can further comprise receiving a second subject-specific genome wide compendium of reads associated with genetic markers from a second biological sample of a subject. The second biological sample can comprise a peripheral blood mononuclear cell sample (PBMC). The second compendium of genetic markers can each comprise a copy number variation (CNV). The method can further comprise filtering artefactual sites from the first and second compendium of reads. The filtering can comprise removing, from the first and second compendium of reads, recurring sites generated over a cohort of reference healthy samples. Alternatively, or in combination, the filtering can comprise identifying shared CNVs between the first and second compendium as germ line mutations and removing said mutations from the first and second compendium of reads. The method can further comprise detecting reads from a third subject-specific genome wide compendium of genetic markers in a third biological sample of the subject to generate a tumor-associated genome-wide representation of genetic markers in the third sample. The method can further comprise normalizing each of the first, second and third compendium of reads to produce a first filtered read set for the first genome-wide compendium of reads, a second filtered read set for the second genome-wide compendium of reads, and a third filtered read set for the third genome-wide compendium of reads. The method can further comprise computing an estimated tumor fraction (eTF) of the third biological samples, using the third filtered read set, by applying a background noise model to one or more integrative mathematical models. The one or more models can be configure to produce a first eTF using the first filtered read set, and/or the one or more models producing a second eTF using the second filtered read set. The method can further comprise detecting a residual disease in the subject if the estimated tumor fraction in the third biological sample exceeds an empirical threshold.

In some embodiments, the disclosure relates to methods for detecting residual disease in a subject in need thereof. Preferably, the residual disease detection comprises detection of minimal residual disease during therapy. Particularly, the disclosure relates to detection of residual disease in one or more of the following settings: (a) after resective surgery; (b) during or after therapy; (c) while monitoring the effectiveness of therapy; (d) while monitoring recurrent or relapse of tumor; or (e) any combination thereof. Especially, the disclosure relates to detection of residual disease during or after chemotherapy, immunotherapy, targeted therapy or a combination thereof, and/or during the course of monitoring the effectiveness of such therapy.

In some embodiments, the disclosure relates to methods for detecting residual disease in a subject in need thereof, comprising, (A) receiving a subject-specific genome wide compendium of genetic markers from a plurality of genetic markers from a biological sample of a subject, the biological sample comprising a tumor sample and optionally a normal cell sample, wherein the compendium of genetic markers is selected from the group consisting of single nucleotide variation (SNV), short insertions and deletions (Indels), copy number variation, structural variants (SV) and combinations thereof; (B) detecting the subject-specific genome wide compendium of genetic markers in a second biological sample of the subject to generate a tumor-associated genome-wide representation of genetic markers in the second sample; (C) filtering artefactual noise markers from the genome-wide compendium of markers by statistically classifying each SNV or Indel in the compendium as signal or noise on the basis of probability of detection of noise (P_{N}) as a function of 1) mapping-quality (MQ) of a read group comprising the SNV, 2) fragment size length of a read group comprising the SNV, 3) consensus test within read duplicate families that comprises the SNV or Indel, 4) base-quality (BQ) of the SNV or Indel; and/or by statistically classifying each CNV or SV window in the compendium as signal or noise on the basis of 1) position thereof relative to the centromere, 2) mapping-quality (MQ) of the read group comprising a CNV or SV window, 3) overlap with a cfDNA mask (blacklist); (D) computing an estimated tumor fraction (eTF) of the biological sample on the basis of one or more integrative mathematical models; and (E) diagnosing a residual disease in the subject based on the estimated tumor fraction and an empirical threshold calculated by background noise model. In some embodiments of the aforementioned methods, (1) for SNV markers, estimated TF (eTF[SNV]) is computed by integrating process-quality metrics comprising estimated genomic coverage and sequencing noise with patient specific parameters comprising mutation load (N); and (2) for CNV markers, estimated TF (eTF[CNV]) is computed by integrating directional depth of coverage skewed in concordance with tumor CNV directionality wherein amplification of copy number is skewed positively and deletion of copy number is skewed negatively. In some embodiments, the BQ, MQ and fragment size filters of the marker are optimized using an ROC curve. In some embodiments, the method comprises employing a combined base quality mapping quality (BQ MQ) filter.

In some embodiments, the residual disease detection method of the disclosure is carried out by receiving a subject-specific genome wide compendium of genetic markers from a plurality of genetic markers from a biological sample comprising a tumor sample of a subject and a normal sample comprising non-tumor sample. In some embodiments, the method includes generating a genome-wide compendium of markers using the subject's tumor sample and the subject's peripheral blood mononuclear cells (PMBC). Particularly, the genome-wide compendium of genetic markers is generated by whole-genome sequencing the subject's sample (e.g., tumor sample) and the control sample (e.g., PMBC). Preferably, the subject's tumor sample comprises a resected tumor, e.g., a solid tumor that is removed post-surgical procedure such as mastectomy; prostatectomy; skin lesion removal; small bowel resection; gastrectomy; thoracotomy; adrenalectomy; colectomy; oophorectomy; thyroidectomy; hysterectomy; glossectomy; or colon polypectomy, preferably thoracotomy.

In some embodiments, the disclosure relates to methods for detecting residual disease in a subject in need thereof, comprising, (A) receiving a subject-specific genome wide compendium of genetic markers from a plurality of genetic markers from a biological sample of a subject, the biological sample comprising a tumor sample and optionally a normal cell sample, wherein the compendium of genetic markers is selected from the group consisting of single nucleotide variation (SNV), short insertions and deletions (Indels), copy number variation, structural variants (SV) and combinations thereof; (B) detecting the subject-specific genome wide compendium of genetic markers in a second biological sample of the subject to generate a tumor-associated genome-wide representation of genetic markers in the second sample; (C) filtering artefactual noise markers from the genome-wide compendium of markers by statistically classifying each SNV or Indel in the compendium as signal or noise on the basis of probability of detection of noise (P_{N}) as a function of 1) mapping-quality (MQ) of a read group comprising the SNV, 2) fragment size length of a read group comprising the SNV, 3) consensus test within read duplicate families that comprises the SNV or Indel, 4) base-quality (BQ) of the SNV or Indel; and/or by statistically classifying each CNV or SV window in the compendium as signal or noise on the basis of 1) position thereof relative to the centromere, 2) mapping-quality (MQ) of the read group comprising a CNV or SV window, 3) overlap with a cfDNA mask (blacklist); (D) computing an estimated tumor fraction (eTF) of the biological sample on the basis of one or more integrative mathematical models; and (E) diagnosing a residual disease in the subject based on the estimated tumor fraction and an empirical threshold calculated by background noise model, wherein the read group comprises a set of reads that cover a specific SNV or indel site, or a set of reads that are included in a specific CNV or SV genomic window. In some embodiments, the normal cell sample comprises PMBC, saliva sample, hair sample, or skin sample. In some embodiments, the subject is a human and the subject's second biological sample comprises a biological material selected from blood, cerebral spinal fluid, pleural fluid, ocular fluid, stool, urine, or a combination thereof.

In some embodiments of the disclosure, the tumor sample comprises a resected tumor or fine-needle aspiration (FNA) sample, snap frozen tissue, optimal cutting temperature compound (OCT)-embedded tissue or formalin-fixed, paraffin-embedded (FFPE) tissue.

In some embodiments of the disclosure, the normal sample comprises peripheral blood mononuclear cells (PMBC), or saliva or skin sample.

In some embodiments of the disclosure, the plurality of genetic markers is received by whole-genome sequencing the subject's biological sample and the control sample.

In some embodiments of the disclosure, the tumor genetic marker compendium comprises high mutation rate and/or high number of SNPs, indels, CNVs or SVs, e.g., at least 1, at least 2, at least 3, at least 5, at least 7, at least 10 or more, e.g., about 15 SNPs or indel per mega base pair or CNV/SV which are at least 5 mega base pair (MBP) in cumulative size, at least 7 MBP, at least 10 MBP or more, e.g., about 15 MBP in cumulative size.

In some embodiments, the disclosure relates to methods for detecting residual disease in a subject in need thereof, comprising, (A) receiving a subject-specific genome wide compendium of genetic markers from a plurality of genetic markers from a biological sample of a subject, the biological sample comprising a tumor sample and optionally a normal cell sample, wherein the compendium of genetic markers is selected from the group consisting of single nucleotide variation (SNV), short insertions and deletions (Indels), copy number variation, structural variants (SV) and combinations thereof; (B) detecting the subject-specific genome wide compendium of genetic markers in a second biological sample of the subject to generate a tumor-associated genome-wide representation of genetic markers in the second sample; (C) filtering artefactual noise markers from the genome-wide compendium of markers by statistically classifying each SNV or Indel in the compendium as signal or noise on the basis of probability of detection of noise (P_{N}) as a function of 1) mapping-quality (MQ) of a read group comprising the SNV, 2) fragment size length of a read group comprising the SNV, 3) consensus test within read duplicate families that comprises the SNV or Indel, 4) base-quality (BQ) of the SNV or Indel; and/or by statistically classifying each CNV or SV window in the compendium as signal or noise on the basis of 1) position thereof relative to the centromere, 2) mapping-quality (MQ) of the read group comprising a CNV or SV window, 3) overlap with a cfDNA mask (blacklist); (D) computing an estimated tumor fraction (eTF) of the biological sample on the basis of one or more integrative mathematical models; and (E) diagnosing a residual disease in the subject based on the estimated tumor fraction and an empirical threshold calculated by background noise model, wherein the empirical noise model is defined by measuring the error rate of detection in normal healthy samples and translated to basal noise eTF estimation.

In some embodiments of the disclosure, the eTF estimation noise threshold is between 0.0001 (10⁻⁴) and 0.000001 (10⁻⁶).

In some embodiments, the disclosure relates to methods for detecting residual disease in a subject in need thereof, comprising, (A) receiving a subject-specific genome wide compendium of somatic genetic markers from a plurality of genetic markers from a biological sample of a subject, the biological sample comprising a tumor sample and a normal cell sample, wherein the compendium of genetic markers is selected from the group consisting of single nucleotide variation (SNV), short insertions and deletions (Indels), copy number variation, structural variants (SV) and combinations thereof, (B) subsequently detecting the subject-specific genome wide compendium of genetic markers in a second biological sample comprising a plasma sample of the subject to generate a tumor-associated genome-wide representation of genetic markers in the second sample; (C) filtering artefactual noise markers from the genome-wide compendium of markers by statistically classifying each SNV or Indel in the compendium as signal or noise on the basis of probability of detection of noise (P_{N}) as a function of 1) mapping-quality (MQ) of a read group comprising the SNV, 2) fragment size length of a read group comprising the SNV, 3) consensus test within read duplicate families that comprises the SNV or Indel, 4) base-quality (BQ) of the SNV or Indel; and/or by statistically classifying each CNV or SV window in the compendium as signal or noise on the basis of 1) position thereof relative to the centromere, 2) mapping-quality (MQ) of the read group comprising a CNV or SV window, 3) overlap with a cfDNA mask (blacklist); (D) computing an estimated tumor fraction (eTF) of the biological sample on the basis of one or more integrative mathematical models; and (E) diagnosing a residual disease in the subject based on the estimated tumor fraction and an empirical threshold calculated by background noise model. In some embodiments, the normal cell sample comprises PMBC, saliva sample, hair sample, or skin sample. In some embodiments, the subject is a human and the subject's second biological sample comprises a biological material selected from blood, cerebral spinal fluid, pleural fluid, ocular fluid, stool, urine, or a combination thereof. In some embodiments, the BQ, MQ and fragment size filters of the marker are optimized using an ROC curve. In some embodiments, the method comprises employing a combined base quality mapping quality (BQ MQ) filter.

In some embodiments, the residual disease detection comprises quantitative estimation of the patient minimal residual disease burden during patient therapy, observation or follow up period. Particularly, the minimal residual disease detection comprises detection of residual disease after resective surgery; detection of residual disease during or after therapy; detection of residual disease to monitor effectiveness of therapy; detection of residual disease to monitor recurrent or relapse of cancer; or a combination thereof. In some embodiments, the minimal residual disease detection comprises detection of residual disease after resective surgery comprising lymph node biopsy; head or neck surgery; uterus or endometrial biopsy; bladder biopsy; mastectomy; prostatectomy; skin lesion removal; small bowel resection; gastrectomy; thoracotomy; adrenalectomy; colectomy; oophorectomy; thyroidectomy; hysterectomy; glossectomy; or colon polypectomy. In some embodiments, the minimal residual disease detection comprises detection of residual disease after therapy comprising chemotherapy, immunotherapy, targeted therapy, radiation therapy or a combination thereof.

In some embodiments of the disclosure, the disease detection method further comprises receiving a plurality of genetic markers from a biological sample of a subject, the biological sample comprising a tumor sample and a normal cell sample, and generating a subject-specific genome wide compendium of genetic markers from the received plurality of genetic markers.

In some embodiments of the disclosure, the disease detection method further comprises detecting the subject-specific genome wide compendium of genetic markers in a second biological sample, e.g., a plasma sample. In some embodiments, the second biological sample is detected in the subject over a course (e.g., 2 days, 1 week, 2 weeks, 1 month, 2 months, 3 months, 4 months, 6 months, 1 year, 18 months, 2 years, 30 months, 3 years, 42 months, 4 years, 5 years 7 years, 10 years, or more, e.g., 15 years or 20 years) to generate a temporally updated representation of tumor genome-wide genetic markers in the patient plasma.

In some embodiments of the disclosure, the disease detection method comprises empirically determining a background noise threshold, wherein a tumor fraction above the background noise threshold provides a quantitative estimation of tumor burden. Particularly, a tumor fraction below the noise threshold is considered non-detected (N.D.).

In some embodiments of the disclosure, the disease detection method comprises quantitative monitoring of a tumor disease (e.g., tumor fraction) over time. In some embodiments, the tumor is brain cancer, lung cancer, skin cancer, nose cancer, throat cancer, liver cancer, bone cancer, lymphomas, pancreatic cancer, skin cancer, bowel cancer, rectal cancer, thyroid cancer, bladder cancer, kidney cancer, mouth cancer, stomach cancer, osteosarcoma or solid state tumor which is heterogeneous or homogeneous in nature. Preferably, the tumor is lung cancer, breast cancer, melanoma, bladder cancer, or osteosarcoma, e.g., lung adenocarcinoma, ductal adenocarcinoma, non-small-cell lung carcinoma lung adenocarcinoma (NSCLC LUAD), cutaneous melanoma, urothelial carcinoma or osteosarcoma.

In some embodiments, the residual disease detection method of the disclosure further comprises: computing an eTF for SNV or indel markers by integrating a probabilistic model including: 1) integrated signal of plasma SNV or indel detection, 2) process-quality metrics comprising estimated genomic coverage and sequencing noise model, 3) patient specific parameters comprising mutation load (N); and/or computing an eTF for CNV or SV markers by utilizing a probabilistic dilution model including: 1) integrating directional depth of coverage skewed between plasma and normal patient samples in concordance with tumor CNV or SV directionality wherein amplification of copy number is skewed positively and deletion of copy number is skewed negatively; 2) integrating the cumulative depth of coverage skewed between tumor and normal (PBMC) patient samples; and 3) finding the dilution ratio between the above signals.

In some embodiments, the residual disease detection method of the disclosure includes (A) receiving a plurality of genetic markers comprising single nucleotide variation (SNV) or copy number variation (CNV) or a combination thereof in a subject's biological sample and a normal cell sample of the subject to generate a subject-specific genome-wide compendium of genetic markers; (B) identifying and filtering artefactual noise markers from the genome-wide compendium of markers, wherein, (1) noise SNVs are identified by statistically classifying each SNV in the compendium as signal or noise on the basis of probability of detection of noise (P_{N}) as a function of base-quality (BQ) of the SNV and mapping-quality (MQ) of the SNV; and/or (2) noise CNVs are identified by statistically classifying each CNV in the compendium as signal or noise on the basis of position thereof relative to the centromere, overlapping a cfDNA mask blacklist thereof in a given depth of coverage and read mappability thereof, (C) computing an estimated tumor fraction (eTF) of the sample on the basis of one or more integrative mathematical models, wherein, for SNV markers, estimated TF (eTF[SNV]) is computed by the mathematical equation eTF[SNV]=1−[1−(M−E(σ)*R)/N]\hat{ }(1/cov), wherein M is the number of tumor-specific compendium detections in the patient sample, a is a measure of empirically-estimated noise, R is the total number of unique reads in a region of interest (ROI), N is tumor mutation load, and cov is the average number of unique reads per site in the ROI; and/or for CNV markers, eTF[CNV] is computed by the mathematical equation eTF[CNV]=(sum_{i}[(P(i)−N(i))*sign[T(i)−N(i)]]−E(sigma))/(sum_{i}[abs(T(i)−N(i))]−E(σ)), wherein P is a median depth value in a genomic window indexed by {i} representing plasma, T is a median depth value in a genomic window indexed by {i} representing tumor, and N is a median depth value in a genomic window indexed by {i} representing normal depth coverage. Particularly under these embodiments, the genomic window for estimating tumor fraction based on the detection of one or more CNV markers is about 500 base pairs (bp).

In some embodiments, the disclosure relates to methods for diagnosing a subject for minimal residual disease, comprising (A) receiving a genome-wide compendium of reads, in the genetic data sequenced from plurality of biological samples received from the subject, the biological samples comprising tumor sample, a normal sample and plasma sample; (B) performing mutation calling on tumor and PBMC samples from the subject comprising MUTECT, LOFREQ and/or STRELKA mutation calling to generate subject-specific reads of somatic SNV (sSNV) or indels as a personalized reference set; (C) collecting and filtering reads from the subject-specific mutation sites comprising (1) removing low mapping quality reads (e.g., <29, ROC optimized); (2) building duplication families (represent multiple PCR/sequencing copies of the same DNA fragment) and producing corrected read based on a consensus test; (3) removing low base quality reads (e.g., <21, ROC optimized); and (4) removing high fragment size reads (e.g., >160, ROC optimized); (D) computing the number of subject-specific mutation sites that have at least one supporting read (in the filtered set) with the exact same substitution as in the tumor; (F) estimating a tumor fraction for SNV based on the mathematical model eTF[SNV]=1−[1−(M−E(σ)*R)/N]\hat{ }(1/cov) . . . . (Equation 1), wherein M is the number of tumor-specific compendium detections in the patient sample, a is a measure of empirically-estimated noise, R is the total number of unique reads in a region of interest (ROI), N is tumor mutation load, and cov is the average number of unique reads per site in the ROI; (G) comparing eTF[SNV] against a detection threshold which comprises an empirically measured basal noise TF estimation from healthy samples, wherein an eTF[SNV] that is above a threshold level (e.g., 2 standard deviations of the noise TF distribution (FPR<2.5%)) is indicative of positive detection; and (K) diagnosing the residual disease in the subject based on the eTF.

In some embodiments, the disclosure relates to methods for diagnosing a subject for minimal residual disease, comprising (A) receiving a genome-wide compendium of reads, in the genetic data sequenced from plurality of biological samples received from the subject, the biological samples comprising tumor sample, a normal sample and plasma sample; (B) performing CNV or SV calling on tumor and PBMC samples from the subject and generating a reference segmentation of a plurality of CNV segments which exceed a threshold length (e.g., >2 Mbp, preferably >5 Mbp) along with annotation of directionality of the segment, wherein amplification is annotated positively and deletion is annotated negatively; (C) collecting single-bp depth coverage information for plasma, tumor and PBMC samples covering the patient specific CNV segmentation region of interest (ROI); (D) dividing the patient specific CNV or SV segmentation ROI to 500 bp windows and calculating the median value per window (artifact suppression) for all samples and window; (E) generating normalized depth coverage information for all 500 bp windows using (a) Robust zscore normalization per sample; and/or (2) Robust Principal Component Analysis (RPCA); (F) Filtering reads/windows from the patient-specific segmentation, wherein filtration comprises: (1) removing low mapping quality reads (e.g., <29, ROC optimized); and/or (2) removing centromere regions (e.g., removing windows with normalized normal value above 10); and/or (3) removing non-represented regions in cfDNA (e.g., removing windows that are not included in a cfDNA representation mask composed from multiple cfDNA samples); (G) Integrating directional depth of coverage skewed between plasma and normal (PBMC) patient samples using the mathematical model sumᵢ[(P(i)−N(i))*sign[T(i)−N(i)]]−E(σ) . . . . (Equation 2), wherein P is a median depth-coverage value in a genomic window indexed by {i} representing plasma depth coverage, normalized by either robust-zscore method or robust PCA compared to a cohort of normal samples; E(sigma) is a measure of empirically-estimated error-rate; T is a median depth value in a genomic window indexed by {i} representing tumor depth coverage, normalized by either robust-zscore method or robust PCA compared to a cohort of normal samples; and N is a median depth value in a genomic window indexed by {i} representing normal depth coverage, normalized by either robust-zscore method or robust PCA compared to a cohort of normal samples; (H) integrating the cumulative depth of coverage skewed between tumor and normal (PBMC) patient samples using the mathematical model sumᵢ[abs(T(i)−N(i))]−E(σ)) . . . . (Equation 3), wherein E(σ) is a measure of empirically-estimated error-rate; T is a median depth value in a genomic window indexed by {i} representing tumor depth coverage, normalized by either robust-zscore method or robust PCA compared to a cohort of normal samples; and N is a median depth value in a genomic window indexed by {i} representing normal depth coverage, normalized by either robust-zscore method or robust PCA compared to a cohort of normal samples; (I) calculating a dilution ratio between directional depth coverage of (G) and cumulative depth coverage (H) which corresponds to the estimated tumor fraction for CNV or SV (eTF[CNV])=(sumᵢ[(P(i)−N(i))*sign[T(i)−N(i)]]−E(σ))/(sumᵢ[abs(T(i)−N(i))]−E(σ)) . . . . (Equation 4); (J) comparing eTF[CNV] against a detection threshold which comprises an empirically measured basal noise TF estimation from healthy samples, wherein an eTF[CNV] that is above a threshold level (e.g., 2 standard deviations of the noise TF distribution (FPR<2.5%)) is indicative of positive detection; and (K) diagnosing the residual disease in the subject based on the eTF.

In some embodiments, the disclosure relates to systems for detecting residual disease in a subject in need thereof, comprising, (A) an analyzing unit configured and arranged to filter artefactual noise markers from a genome-wide compendium of markers, wherein the genome-wide compendium of markers is generated from a plurality of genetic markers from a biological sample of a subject, the biological sample comprising a tumor sample and a normal cell sample, wherein the compendium of genetic markers is selected from the group consisting of single nucleotide variation (SNV), indels, copy number variation, SV and combinations thereof, the analyzing unit further comprising detecting the subject-specific genome wide compendium of genetic markers in a second biological sample comprising a plasma sample of the subject to generate a representation of tumor genome-wide genetic markers in the patient plasma, the analyzing unit further comprising engines selected from the group consisting of an SNV and indel classification engine, a CNV and SV classification engine, and combinations thereof, wherein: the SNV and indel classification engine statistically classifies each SNV in the compendium as signal or noise on the basis of probability of detection of noise (P_{N}) as a function of 1) mapping-quality (MQ) of the read group comprises the SNV or Indel, 2) fragment size length of the read group comprises the SNV or Indel, 3) consensus test within read duplicate families that comprises the specific SNV, 4) base-quality (BQ) of the SNV or Indel, and the CNV and SV classification engine statistically classifies each CNV or SV window in the compendium as signal or noise on the basis of 1) position thereof relative to the centromere, 2) mapping-quality (MQ) of the read group comprises the CNV or SV window, 3) Representation of the CNV or SV window in cfDNA data; (B) an eTF unit configured and arranged to calculate estimated tumor fraction (eTF) of the sample on the basis of one or more integrative mathematical models; and (C) a display unit that outputs a residual disease profile of the subject based on the estimated tumor fraction.

In some embodiments of the aforementioned systems of the disclosure, the eTF unit is further configured and arranged to: compute an eTF for SNV or Indel markers by integrating a probabilistic model comprising: 1) integrated signal of plasma SNV or Indel detection, 2) process-quality metrics comprising estimated genomic coverage and sequencing noise model, 3) patient specific parameters comprising mutation load (N); and/or computing an eTF for CNV or SV markers by utilizing a probabilistic mixture model including: 1) integrating directional depth of coverage skewed between plasma and normal patient samples in concordance with tumor CNV or SV directionality wherein amplification of copy number is skewed positively and deletion of copy number is skewed negatively; 2) integrating the cumulative depth of coverage skewed between tumor and normal patient samples; and 3) finding a dilution ratio between the above signals.

In some embodiments of the aforementioned systems of the disclosure, the tumor fraction estimating unit (B) comprises a processor, the processor configured to execute the computer-readable instructions, which when executed, carry out the method for estimating tumor fraction (eTF) of the sample on the basis of one or more of the following integrative mathematical models (1) eTF[SNV]=1−[1−(M−E(σ)*R)/N]\hat{ }(1/cov), wherein M is the number of tumor-specific SNV compendium detections in the patient plasma sample, a is a measure of empirically-estimated error-rate, R is the total number of unique reads in the SNV compendium region of interest (ROI), N is tumor mutation load, and cov is the average number of unique reads per site in the SNV compendium ROI; and/or (2) eTF[CNV]=(sum_{i}[(P(i)−N(i))*sign[T(i)−N(i)]]−E(sigma))/(sum_{i}[abs(T(i)−N(i))]−E(σ)), wherein P is a median depth-coverage value in a genomic window indexed by {i} representing plasma depth coverage, normalized by either robust-zscore method or robust PCA compared to a cohort of normal samples; T is a median depth value in a genomic window indexed by {i} representing tumor depth coverage, normalized by either robust-zscore method or robust PCA compared to a cohort of normal samples; N is a median depth value in a genomic window indexed by {i} representing normal depth coverage, normalized by either robust-zscore method or robust PCA compared to a cohort of normal samples, wherein {i} is a discrete index counting all the genomic windows that cover the patient tumor-specific amplification and deletion genomic segments.

In some embodiments, the disclosure relates to computer readable media comprising computer-executable instructions, which, when executed by a processor, cause the processor to carry out a method or a set of steps for detection of residual disease, the method or steps comprising, (A) receiving a subject-specific genome wide compendium of genetic markers from a plurality of genetic markers from a biological sample of a subject, the biological sample comprising a tumor sample and optionally a normal cell sample, wherein the compendium of genetic markers is selected from the group consisting of single nucleotide variation (SNV), short insertions and deletions (Indels), copy number variation, structural variants (SV) and combinations thereof, (B) detecting the subject-specific genome wide compendium of genetic markers in a second biological sample of the subject to generate a tumor-associated genome-wide representation of genetic markers in the second sample; (C) filtering artefactual noise markers from the genome-wide compendium of markers by statistically classifying each SNV or Indel in the compendium as signal or noise on the basis of probability of detection of noise (P_{N}) as a function of 1) mapping-quality (MQ) of a read group comprising the SNV, 2) fragment size length of a read group comprising the SNV, 3) consensus test within read duplicate families that comprises the SNV or Indel, 4) base-quality (BQ) of the SNV or Indel; and/or by statistically classifying each CNV or SV window in the compendium as signal or noise on the basis of 1) position thereof relative to the centromere, 2) mapping-quality (MQ) of the read group comprising a CNV or SV window, 3) overlap with a cfDNA mask (blacklist); (D) computing an estimated tumor fraction (eTF) of the biological sample on the basis of one or more integrative mathematical models; and (E) diagnosing a residual disease in the subject based on the estimated tumor fraction and an empirical threshold calculated by background noise model.

The disclosure additionally relates to a method for cancer stratification comprising detection of minimal residual disease (MRD) in a cancer patient. The stratification method comprises identifying low-abundance MRD-specific markers in accordance with the aforementioned methods; and detecting the markers to diagnose MRD. The cancer stratification method may further include detection of tumor by methods such as RT-PCR of lung cancer specific markers and/or molecular imaging using probes.

### BRIEF DESCRIPTION OF THE DRAWINGS

The details of one or more embodiments of the disclosure are set forth in the accompanying drawings/tables and the description below. Other features, objects, and advantages of the disclosure will be apparent from the drawings/tables and detailed description, and from the claims. FIG. 1A shows a schematic representation of the diagnostic methods of the instant disclosure, e.g., for detecting minimal residual tumor disease, in accordance with various embodiments. FIG. 1B shows a representative workflow for detecting residual disease in a subject, in accordance with various embodiments. FIG. 1C shows a representative workflow for detecting residual disease in a subject, in accordance with various embodiments. FIG. 1D shows a representative workflow of the present disclosure for diagnosing minimal residual disease (MRD) in a subject based on measurement of single nucleotide polymorphisms or indels. FIG. 1E shows a representative workflow of the present disclosure for diagnosing minimal residual disease (MRD) in a subject based on measurement of copy number variations or structural variations.

FIG. 2A-2B shows charts of detection probabilities based on the extrinsic or intrinsic parameters. FIG. 2A shows detection probability for various tumor fraction and coverage (up to the genomic equivalent limitation: ˜1000 molecules) based on the Bernoulli model. FIG. 2B shows detection probability for genome wide SNV integration (Binomial model), assuming the integration of 20,000 point mutations.

FIG. 3A-3K shows the effect of applying a various filters, in accordance with various embodiments and the estimation of tumor fractions that are provided by the instant methods. FIG. 3A shows the effect of applying a Base-Quality (BQ) filter. FIG. 3B shows the effect of optimizing base-quality filtration by receiver operating curve (ROC). FIG. 3C shows the effect of applying a joint Base Quality (BQ) and Mapping-Quality (MQ) optimized filter in evaluating the error rate distribution across multiple replicates using control samples, which provides for about 7-fold change (FC) suppression in sequencing error. Pre-filter noise shows a rate of ˜2×10³ for both lung and melanoma cancer types, post filter noise rate decrease to ˜2×10⁴ for both cancer types. FIG. 3D shows the effect of applying a joint Base Quality (BQ) and Mapping-Quality (MQ) optimized filter with alleviated 35× coverage. The filter permits detection of markers in samples having a TF as low as 1/20,000. Red line represents theoretical (binomial model) expectation and empirical measurements are shown in black (mean & confidence interval for 5 independent replicates. Noise level is represented by the gray area according to TF=0 detection distribution. FIG. 3E shows in silico validation of TF estimation in melanoma samples. Input mixture TF (x-axis) versus TF estimated from the mutation pattern (y-axis) indicating a high correlation (R²=0.999). Accurate and specific estimations were obtained to all TF above 5×10⁻⁵. FIG. 3F and FIG. 3G show diagnostic methods, in accordance with various embodiments, which permit detection of signatures of genetic biomarkers in other types of solid tumors, e.g., lung tumor fraction (FIG. 3F) and breast cancer patients (FIG. 3G) even in tumor fractions (TF) as low as 1/10000. FIG. 3H shows reliable sSNV-based tumor fraction estimation with tumor fraction (TF) as low as 5×10⁻⁵. FIG. 3I shows reliable sCNV-based tumor fraction estimation with tumor fraction (TF) as low as 5×10⁻⁵, preferably at TF>10⁻⁴. FIG. 3J shows strong correlation between estimation of TF using SNV-based estimation (x-axis) and CNV-based estimation (y-axis). The grey quadrant shows weaker correlation between SNV-based estimation and SNV-based estimation at TF below a threshold value of 5×10⁻⁵. FIG. 3K shows a box plot showing comparison of the instant methods compared to ICHOR-CNA method.

FIG. 4 shows the SNV detection rate in background noise model (healthy PBMC and cfDNA samples) alongside of 2 cancer patient (BB1122, BB1125) cfDNA samples taken prior resective surgery (pre-op) and after resective surgery (post-op) and 2 healthy control cfDNA samples (BB600 and BB601), in accordance with various embodiments.

FIG. 5A and FIG. 5B show clinical assessment of patient samples using the systems and methods of the disclosure. FIG. 5A shows the exemplary evaluation of the systems and methods of the disclosure using clinical samples obtained from subjects with early-stage lung cancer and/or minimal residual disease (MRD) patients, in accordance with various embodiments. The data show tumor fraction (TF) estimation for pre-surgery and post-surgery plasma samples across all patients analyzed. Only two patients show post-surgery TF above the noise threshold of 5×10⁻⁵. However, all healthy control samples show TF below the detection threshold. N.D. denotes not detected. The data shows concordant results with the SNV method in terms of plasma detection and TF correlation. FIG. 5B shows calculation of zscores across 11 different samples obtained from patients with adenocarcinoma. The data show that the zscores of healthy controls are below the threshold level (e.g., zscore of 2, as indicated by the horizontal dotted line). FIG. 5C shows calculation of zscores across 11 different samples obtained from patients with adenocarcinoma, as compared to cross-patient negative controls. The data show that the zscores of healthy controls are below the threshold level (e.g., zscore of 2, as indicated by the horizontal dotted line). A concordance between sSNV-based and sCNV-based detection methods was observed (FIG. 5D).

FIG. 6A-6E shows analytic approach to integrate large number of directional depth coverage skews across large genomic CNV segments. FIG. 6A shows the integration of sparse CNV skew at TF=0.001, wherein the upper panel shows comparison of the single-bp depth-coverage between synthetic plasma (TF=10⁻³) and matched PBMC in a 10 Kbp segment of an amplification; the middle panel shows the residuals between the plasma and PBMC and the lower panel shows sum of residuals. In the middle panel, note the sparse but positive bias of the residual and in the lower panel, partly due to the amplification positive bias the sum of residuals, (signal) is accumulating when integrated over the genome. FIG. 6B shows a profile of the tumor read-depth (red), germline read-depth (pink) and pre-surgery plasma cfDNA read-depth (blue) in a representative amplified segment. Pre-surgery plasma shows read depth comparable to the germline DNA, but also shows amplified depth skew at the telomeric end of the amplified segment. The mathematical method integrates read depth skews across the genome as described. FIG. 6C shows signal-to-noise (SNR) for each TF, where all TFs above 10⁻⁶ show positive (>0) SNR detection (demonstrating high sensitivity). FIG. 6D shows CNV plasma SNR is linear to TF (dilution model), show similar dynamic for lung/melanoma/breast patients. FIG. 6E shows a chart of skew versus tumor fraction (TF) when taking neutral regions of the genome (e.g., regions that do not contain amplification and/or deletion). As can be seen, in these regions, the depth coverage skew between plasma and PBMC is not biased, and the probability for positive and negative skew is similar. Therefore there is no signal and the SNR=0, regardless of the TF (x-axis).

FIG. 7A-FIG. 7C provide schematic representations of systems of the present disclosure, in accordance with various embodiments.

FIG. 8 provides a representative flowchart outlining the identification and/or classification of post-surgery cancer subjects as candidates for adjuvant therapy, in accordance with various embodiments.

FIG. 9 shows illustrates a comparison between patient-specific sSNV integration of the various embodiments herein, versus ICHOR (Broad Institute). In particular, sensitivity of detection is increased by about 100-fold compared to MIT-Broad Institute's ICHOR detection method.

FIG. 10A-FIG. 10E show use of orthogonal features such as fragment size in the diagnostic methods of the disclosure and the concomitant effects of application of such orthogonal features in SNV-based methods. FIG. 10A shows fragment size distribution shown in healthy normal cfDNA sample. FIG. 10B shows a fragment size shift in breast tumor cfDNA (red and purple) show compared to normal cfDNA sample. FIG. 10C shows that in mouse xenograft (PDX) models, circulating DNA from the tumor origin is significantly shorter than circulating DNA that is from normal origin. FIG. 10D shows a line graph of the fragment DNA size (x-axis; number of bases) plotted against frequency of observing a fragment of said length across tumor and normal samples. FIG. 10E shows patient-specific mutation detections using orthogonal features such as correspondence of DNA fragments with tumor origin based on their fragment size distribution (x-axis) and the GMM joint log odds ratio (y-axis).

FIG. 11A-FIG. 11J show use of orthogonal features such as fragment size in the diagnostic methods of the disclosure and the concomitant effects of application of such orthogonal features in CNV-based methods. FIG. 11A shows a line graph of genomic region (bp) versus cumulative plasma depth coverage skew (bottom panel), plasma-vs-normal depth coverage skew (middle panel) and coverage (top panel). FIG. 11B shows relationship between the log 2 of the depth coverage (log 2>0.5=amplification, log 2<−0.5=deletion) and the local fragment size center-of-mass (COM) in that segment. FIG. 11C shows a relationship between depth coverage based CNV detection and fragment size center-of-mass (COM) based CNV detection in patient samples. FIG. 11D shows lack of a relationship between depth coverage based CNV detection and fragment size center-of-mass (COM) based CNV detection in normal (healthy) plasma samples. FIG. 11E and FIG. 11F show changes in COM, absolute slope value and R² in two patients undergoing therapy. Values are shown at baseline (day 0) and at 21-days and 42-days post-treatment. FIG. 11G shows a relationship between fragment size log 2 slopes and tumor fractions in patients. FIG. 11H shows results of a clinical study in cancer patients examining an association between relapse-free time and detection (zscore) of tumor DNA post-surgery (2 weeks after surgery). FIG. 11I shows bar charts of tumor fractions of four patients at baseline (day 0), midpoint (day 21) and end (day 42) of therapy. FIG. 11J shows bar charts of normalized CNV scores of four patients at baseline (day 0), midpoint (day 21) and end (day 42) of therapy.

### DETAILED DESCRIPTION

The following description of various embodiments is exemplary and explanatory only and is not to be construed as limiting or restrictive in any way. Other embodiments, features, objects, and advantages of the present teachings will be apparent from the description and accompanying drawings, and from the claims.

Unless otherwise defined, scientific and technical terms used in connection with the present teachings described herein shall have the meanings that are commonly understood by those of ordinary skill in the art. The terminology used in the description of the disclosure herein is for the purpose of describing particular embodiments only and is not intended to be limiting of the disclosure. Further, unless otherwise required by context, singular terms shall include pluralities and plural terms shall include the singular. Generally, nomenclatures utilized in connection with, and techniques of molecular biology, and protein and oligo- or polynucleotide chemistry and hybridization described herein are those well-known and commonly-used in the art. Standard techniques are used, for example, for nucleic acid purification and preparation, chemical analysis, recombinant nucleic acid, and oligonucleotide synthesis. Enzymatic reactions and purification techniques are performed according to manufacturer's specifications or as commonly accomplished in the art or as described herein. The techniques and procedures described herein are generally performed according to conventional methods well known in the art and as described in various general and more specific references that are cited and discussed throughout the instant specification. See, e.g., Sambrook et al., Molecular Cloning: A Laboratory Manual (Third ed., Cold Spring Harbor Laboratory Press, Cold Spring Harbor, N.Y. 2000). The nomenclatures utilized in connection with, and the laboratory procedures and techniques described herein are those well-known and commonly-used in the art.

The various embodiments of the present disclosure are further described in detail in the paragraphs below.

As used in the description of the disclosure and the appended claims, the singular forms “a,” “an,” and “the” are intended to include the plural forms as well, unless the context clearly indicates otherwise. Also as used herein, “and/or” refers to and encompasses any and all possible combinations of one or more of the associated listed items, as well as the lack of combinations when interpreted in the alternative (“or”).

The word “about” means a range of plus or minus 10% of that value, e.g., “about 5” means 4.5 to 5.5, “about 100” means 90 to 100, etc., unless the context of the disclosure indicates otherwise, or is inconsistent with such an interpretation. For example in a list of numerical values such as “about 49, about 50, about 55”, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

Where a range of values is provided in this disclosure, it is intended that each intervening value between the upper and lower limit of that range and any other stated or intervening value in that stated range is encompassed within the disclosure. For example, if a range of 1 μM to 8 μM is stated, it is intended that 2 μM, 3 μM, 4 μM, 5 μM, 6 μM, and 7 μM are also explicitly disclosed.

As used herein, the term “plurality” can be 2, 3, 4, 5, 6, 7, 8, 9, 10, or more.

As used herein, the term “detecting,” refers to the process of determining a value or set of values associated with a sample by measurement of one or more parameters in a sample, and may further comprise comparing a test sample against reference sample. In accordance with the present disclosure, the detection of tumors includes identification, assaying, measuring and/or quantifying one or more markers.

As used herein, the term “diagnosis” refers to methods by which a determination can be made as to whether a subject is likely to be suffering from a given disease or condition, including but not limited diseases or conditions characterized by genetic variations. The skilled artisan often makes a diagnosis on the basis of one or more diagnostic indicators, e.g., a marker, the presence, absence, amount, or change in amount of which is indicative of the presence, severity, or absence of the disease or condition. Other diagnostic indicators can include patient history; physical symptoms, e.g., unexplained weight loss, fever, fatigue, pains, or skin anomalies; phenotype; genotype; or environmental or heredity factors. A skilled artisan will understand that the term “diagnosis” refers to an increased probability that certain course or outcome will occur; that is, that a course or outcome is more likely to occur in a patient exhibiting a given characteristic, e.g., the presence or level of a diagnostic indicator, when compared to individuals not exhibiting the characteristic. Diagnostic methods of the disclosure can be used independently, or in combination with other diagnosing methods, to determine whether a course or outcome is more likely to occur in a patient exhibiting a given characteristic.

The term “normal” as used in the context of “normal cell,” is meant to refer to a cell of an untransformed phenotype or exhibiting a morphology of a non-transformed cell of the tissue type being examined (e.g., PBMC). In some embodiments, “normal sample” as used herein includes non-tumor sample, e.g., saliva sample, skin sample, hair sample or the like. It should be noted that the methods of the disclosure may be implemented without the use of normal samples.

The term “abnormal,” as used herein, generally refers to a state of a biological system that deviates in some degree from normal (e.g., wild-type). Abnormal states can occur at the physiological or molecular level. Representative examples include, e.g., physiological state (disease, pathology) or a genetic aberration (mutation, single nucleotide variant, copy number variant, gene fusion, indel, etc.). A disease state can be cancer or pre-cancer. An abnormal biological state may be associated with a degree of abnormality (e.g., a quantitative measure indicating a distance away from normal state).

The term “likelihood,” as used herein, generally refers to a probability, a relative probability, a presence or an absence, or a degree.

As used herein, the term “tumor” includes any cell or tissue that may have undergone transformation at the genetic, cellular, or physiological level compared to a normal or wild-type cell. The term usually denotes neoplastic growth which may be benign (e.g., a tumor which does not form metastases and destroy adjacent normal tissue) or malignant/cancer (e.g., a tumor that invades surrounding tissues, and is usually capable of producing metastases, may recur after attempted removal, and is likely to cause death of the host unless adequately treated). See Steadman's Medical Dictionary, 28^{th} Ed Williams & Wilkins, Baltimore, MD (2005).

The term “cancer” (used interchangeably with “tumor”) refers to human cancers and carcinomas, sarcomas, adenocarcinomas, lymphomas, leukemia, solid and lymphoid cancers, etc. Examples of different types of cancer include, but are not limited to, lung cancer, pancreatic cancer, breast cancer, gastric cancer, bladder cancer, oral cancer, ovarian cancer, thyroid cancer, prostate cancer, uterine cancer, testicular cancer, neuroblastoma, squamous cell carcinoma of the head, neck, cervix and vagina, multiple myeloma, soft tissue and osteogenic sarcoma, colorectal cancer, liver cancer, renal cancer (e.g., RCC), pleural cancer, cervical cancer, anal cancer, bile duct cancer, gastrointestinal carcinoid tumors, esophageal cancer, gall bladder cancer, small intestine cancer, cancer of the central nervous system, skin cancer, choriocarcinoma; osteogenic sarcoma, fibrosarcoma, glioma, melanoma, etc. In some embodiments, “liquid” cancers, e.g., blood cancers such as lymphoma and/or leukemia are excluded.

Exemplary cancers include, but are not limited to, adrenocortical carcinoma, AIDS-related cancers, AIDS-related lymphoma, anal cancer, anorectal cancer, cancer of the anal canal, appendix cancer, childhood cerebellar astrocytoma, childhood cerebral astrocytoma, basal cell carcinoma, skin cancer (non-melanoma), biliary cancer, extrahepatic bile duct cancer, intrahepatic bile duct cancer, bladder cancer, urinary bladder cancer, bone and joint cancer, osteosarcoma and malignant fibrous histiocytoma, brain cancer, brain tumor, brain stem glioma, cerebellar astrocytoma, cerebral astrocytoma/malignant glioma, ependymoma, medulloblastoma, supratentorial primitive neuroectodeimal tumors, visual pathway and hypothalamic glioma, breast cancer, bronchial adenomas/carcinoids, carcinoid tumor, gastrointestinal, nervous system cancer, nervous system lymphoma, central nervous system cancer, central nervous system lymphoma, cervical cancer, childhood cancers, chronic lymphocytic leukemia, chronic myelogenous leukemia, chronic myeloproliferative disorders, colon cancer, colorectal cancer, cutaneous T-cell lymphoma, lymphoid neoplasm, mycosis fungoides, Seziary Syndrome, endometrial cancer, esophageal cancer, extracranial germ cell tumor, extragonadal germ cell tumor, extrahepatic bile duct cancer, eye cancer, intraocular melanoma, retinoblastoma, gallbladder cancer, gastric (stomach) cancer, gastrointestinal carcinoid tumor, gastrointestinal stromal tumor (GIST), germ cell tumor, ovarian germ cell tumor, gestational trophoblastic tumor glioma, head and neck cancer, hepatocellular (liver) cancer, Hodgkin lymphoma, hypopharyngeal cancer, intraocular melanoma, ocular cancer, islet cell tumors (endocrine pancreas), Kaposi's sarcoma, kidney cancer, renal cancer, laryngeal cancer, acute lymphoblastic leukemia, acute myeloid leukemia, chronic lymphocytic leukemia, chronic myelogenous leukemia, hairy cell leukemia, lip and oral cavity cancer, liver cancer, lung cancer, non-small cell lung cancer, small cell lung cancer, AIDS-related lymphoma, non-Hodgkin lymphoma, primary central nervous system lymphoma, Waldenstram macroglobulinemia, medulloblastoma, melanoma, intraocular (eye) melanoma, Merkel cell carcinoma, mesothelioma malignant, mesothelioma, metastatic squamous neck cancer, mouth cancer, cancer of the tongue, multiple endocrine neoplasia syndrome, mycosis fungoides, myelodysplastic syndromes, myelodysplastic/myeloproliferative diseases, chronic myelogenous leukemia, acute myeloid leukemia, multiple myeloma, chronic myeloproliferative disorders, nasopharyngeal cancer, neuroblastoma, oral cancer, oral cavity cancer, oropharyngeal cancer, ovarian cancer, ovarian epithelial cancer, ovarian low malignant potential tumor, pancreatic cancer, islet cell pancreatic cancer, paranasal sinus and nasal cavity cancer, parathyroid cancer, penile cancer, pharyngeal cancer, pheochromocytoma, pineoblastoma and supratentorial primitive neuroectodermal tumors, pituitary tumor, plasma cell neoplasm/multiple myeloma, pleuropulmonary blastoma, prostate cancer, rectal cancer, renal pelvis and ureter, transitional cell cancer, retinoblastoma, rhabdomyosarcoma, salivary gland cancer, Ewing family of sarcoma tumors, Kaposi Sarcoma, uterine cancer, uterine sarcoma, skin cancer (non-melanoma), skin cancer (melanoma), merkel cell skin carcinoma, small intestine cancer, soft tissue sarcoma, squamous cell carcinoma, stomach (gastric) cancer, supratentorial primitive neuroectodermal tumors, testicular cancer, throat cancer, thymoma, thymoma and thymic carcinoma, thyroid cancer, transitional cell cancer of the renal pelvis and ureter and other urinary organs, gestational trophoblastic tumor, urethral cancer, endometrial uterine cancer, uterine sarcoma, uterine corpus cancer, vaginal cancer, vulvar cancer, and Wilm's Tumor.

As used herein, the term “non-small cell lung carcinoma” or NSCLC as used herein refers to all lung cancers that are not small cell lung cancer and includes several sub-types including but not limited to large cell carcinoma, squamous cell carcinoma and adenocarcinoma. All stages and metastasis are included. Accounting for 25% of lung cancers, squamous cell carcinoma usually starts near a central bronchus. A hollow cavity and associated necrosis are commonly found at the center of the tumor. Well-differentiated squamous cell cancers often grow more slowly than other cancer types. Adenocarcinoma accounts for 40% of non-small cell lung cancers. It usually originates in peripheral lung tissue. Most cases of adenocarcinoma are associated with smoking; however, among people who have never smoked, adenocarcinoma is the most common form of lung cancer. See, Rosell et al., Lung Cancer, 46(2), 135-48, 2004; Coate et al., Lancet Oncol, 10, 1001-10, 2009.

As used herein, the term “residual disease” refers to the persistence of residual neoplastic cells even after intervention, e.g., surgical intervention, radiological ablation, chemotherapy, or the like. The term “minimal residual disease” (MRD) describes the situation in which, after therapy (e.g., chemotherapy, immunotherapy or targeted therapy) for a tumor, a morphologically normal tissue (e.g., lung tissue) can still harbor a relevant amount of residual malignant cells. Detection of minimal residual disease (MRD) is a new practical tool for a more exact measurement of remission induction during therapy. In the context of liquid tumors (e.g., lymphoma or myeloma), the term MRD may relate to a limit of detection below 10⁻⁴, e.g., 10⁻⁵, or even 10⁻⁶. In the context of solid tumors, the term “minimal residual disease” may relate to situations in which tumor markers are below what is detectable using traditional means of detection, e.g., ctDNA detection or plasma DNA analysis. In some embodiments, MRD relates to situations wherein fewer than 100 copies, preferably fewer than 40 copies, and particularly fewer than 10 copies of ctDNA are detected per 5 ml of plasma (Bettegowda et al., Sci Transl Med., 6(224), 224ra24, 2014).

As used herein, the term “subject” means a mammalian animal, including a human, a veterinary or farm animal, a domestic animal or pet, and animals normally used for clinical research. Particularly, the subject is a human subject, e.g., a human patient diagnosed with a tumor or suspected of having a tumor. A subject may have, potentially have, or be suspected of having one or more characteristics selected from cancer, a symptom(s) associated with cancer, asymptomatic with respect to cancer or undiagnosed (e.g., not diagnosed for cancer). The subject may have cancer, the subject may show a symptom(s) associated with cancer, the subject may be free from symptoms associated with cancer, or the subject may not be diagnosed with cancer. In some embodiments, the subject is a human.

As used herein, the term “single nucleotide polymorphism” or “single nucleotide variation” (“SNP” or “SNV”) in reference to a mutation refers to a difference of at least one nucleotide in a sequence in comparison to another sequence.

The term “copy number variation” or “CNV” refers to a comparative numerical change in the presence or absence/gain or loss, of gene fragments having the same nucleotide sequence. In human genomes, copy number variants can involve homozygous or heterozygous duplications or multiplications of one or more sections of DNA, or homozygous or heterozygous deletions of one or more sections of DNA. Directionality of CNV is usually denoted positively for duplications/multiplications of CNVs and negatively for deletions of CNVs.

As used herein, the term “indel” refers to a location on a genome where one or more bases are present in one allele, with no bases present in another allele. Insertions or deletions are distinct from an evolutionary point of view, but during analysis such as described herein, they are often not distinguished as an insertion in one allele is equivalent to a deletion in the other allele. Thus the term indel is to refer to the location of the insertion/deletion between two alleles.

As used herein, the term “structural variant” refers to changes in some parts of the chromosomes instead of changes in the number of chromosomes or sets of chromosomes in the genome. There are four common types of mutations which result in structural variants: deletions and insertions, for example duplications (involving a change in the amount of DNA in a chromosome, loss and gain of genetic material, respectively), inversions (involving a change in the arrangement of a chromosomal segment) and translocations (involving a change in the location of a chromosomal segment which can give rise to gene fusions). In the present invention, the term “structural variant” includes loss of genetic material, a gain of genetic material, a translocation, a gene fusion and combinations thereof.

The term “sample” as used herein refers to a composition that is obtained or derived from a subject of interest that contains a cellular and/or other molecular entity that is to be characterized and/or identified, for example based on physical, biochemical, chemical and/or physiological characteristics. Preferably, the sample is a “biological sample,” which means a sample that is derived from a living entity, e.g., cells, tissues, organs and the like. In some embodiments, the source of the tissue sample may be blood or any blood constituents; bodily fluids; solid tissue as from a fresh, frozen and/or preserved organ or tissue sample or biopsy or aspirate; and cells from any time in gestation or development of the subject or plasma. Samples include, but not limited to, primary or cultured cells or cell lines, cell supernatants, cell lysates, platelets, serum, plasma, vitreous fluid, ocular fluid, lymph fluid, synovial fluid, follicular fluid, seminal fluid, amniotic fluid, milk, whole blood, urine, cerebrospinal fluid (CSF), saliva, sputum, tears, perspiration, mucus, tumor lysates, and tissue culture medium, as well as tissue extracts such as homogenized tissue, tumor tissue, and cellular extracts. Samples further include biological samples that have been manipulated in any way after their procurement, such as by treatment with reagents, solubilized, or enriched for certain components, such as proteins or nucleic acids, or embedded in a semi-solid or solid matrix for sectioning purposes, e.g., a thin slice of tissue or cells in a histological sample. Samples may contain environmental components, such as, e.g., water, soil, mud, air, resins, minerals, etc. In certain embodiments, a sample may comprise biological sample containing DNA (e.g., gDNA), RNA (e.g., mRNA, tRNA), protein, or combinations thereof, obtained from a subject (e.g., human or other mammalian subject).

As used herein, the term “cell” is used interchangeably with the term “biological cell.” Non-limiting examples of biological cells include eukaryotic cells, plant cells, animal cells, such as mammalian cells, reptilian cells, avian cells, fish cells, or the like, prokaryotic cells, bacterial cells, fungal cells, protozoan cells, or the like, cells dissociated from a tissue, such as muscle, cartilage, fat, skin, liver, lung, neural tissue, and the like, immunological cells, such as T cells, B cells, natural killer cells, macrophages, and the like, embryos (e.g., zygotes), oocytes, ova, sperm cells, hybridomas, cultured cells, cells from a cell line, cancer cells, infected cells, transfected and/or transformed cells, reporter cells, and the like. A mammalian cell can be, for example, from a human, a mouse, a rat, a horse, a goat, a sheep, a cow, a primate, or the like.

As used herein, the term “marker” refers to a characteristic that can be objectively measured as an indicator of normal biological processes, pathogenic processes or a pharmacological response to a therapeutic intervention, e.g., treatment with an anti-cancer agent. Representative types of markers include, for example, molecular changes in the structure (e.g., sequence) or number of the marker, comprising, e.g., gene mutations, gene duplications, or a plurality of differences, such as somatic alterations in cfDNA, copy number variations, tandem repeats, or a combination thereof.

As used herein the term “genetic marker” refers to a sequence of DNA that has a specific location on a chromosome that can be measured in a laboratory. The term “genetic marker” can also be used to refer to, e.g., a cDNA and/or an mRNA encoded by a genomic sequence, as well as to that genomic sequence itself. Genetic markers may include two or more alleles or variants. Genetic markers may be direct (e.g., located within the gene or locus of interest (e.g., candidate gene)), indirect (e.g., closely linked with the gene or locus of interest, e.g., due to proximity to but not within the gene or locus of interest). Moreover, genetic markers may also be unrelated to the genes or loci, e.g., SNVs, CNVs, indels, SVs, or tandem repeats, which are present in non-coding segments of the genome. Genetic markers include nucleic acid sequences which either do or do not code for a gene product (e.g., a protein). Particularly, the genetic markers include single nucleotide polymorphisms/variations (SNPs/SNVs) or copy number variations (CNVs) or a combination thereof. Preferably, the genetic marker includes somatic variations in the DNA, e.g., sSNV or sCNV, indels, SVs, or a combination thereof compared to a reference sample.

As used herein, the term “cell free DNA” or “cfDNA” refers to strands of deoxyribose nucleic acids (DNA) found free of cells, for example, as extracted or isolated from plasma/serum of circulating blood, extracted from lymph, cerebrospinal fluid (CSF), urine or other bodily fluids. The term “cfDNA” is contrasted with “circulating tumor DNA” or “ctDNA.” Cell-free DNA (cfDNA) is a broader term which describes DNA that is freely circulating in the bloodstream, but is not necessarily of tumor origin.

As used herein, the term “germline DNA” or “gDNA” refers to DNA isolated or extracted from a patient's peripheral mononuclear blood cells, including lymphocytes that are in turn obtained from circulating blood.

As used herein, the term “variation” refers to a change or deviation. In reference to nucleic acid, a variation refers to a difference(s) or a change(s) between DNA nucleotide sequences, including differences in copy number (CNVs). This actual difference in nucleotides between DNA sequences may be an SNP, and/or a change in a DNA sequence, e.g., fusion, deletion, addition, repeats, etc., observed when a sequence is compared to a reference, such as, e.g., germline DNA (gDNA) or a reference human genome HG38 sequence. Preferably, the variation refers to difference between cfDNA sequence and a control DNA sequence that is not from a tumor cell, such as when cfDNA is compared to reference HG38 sequence; when cfDNA is compared to gDNA. Differences identified in both gDNA and cfDNA are considered “constitutional” and may be ignored.

The term “control,” as used herein, refers to a reference for a test sample, such as control DNA isolated from peripheral mononuclear blood cells and lymphocytes, where these cells are not cancer cells, and the like. A “reference sample,” as used herein, refers to a sample of tissue or cells that may or may not have cancer that are used for comparisons. Thus a “reference” sample thereby provides a basis to which another sample, for example plasma sample containing cfDNA can be compared. In contrast, a “test sample” refers to a sample compared to a reference sample or control sample. The reference sample need not be cancer free, such as when a reference sample and a test sample are obtained from the same patient separated by time.

In some embodiments, the reference sample or control may comprise a reference assembly. The term “reference assembly” refers to a digital nucleic acid sequence database, such as the human genome (HG38) database containing HG38 assembly sequences (assembled: December 2013). The gateway can be accessed through the Human (Homo sapiens) University of California Santa Cruz (UCSC) Genome Browser Gateway at the world-wide-web URL GENOME(dot)UCSC(dot)EDU. Alternately, the reference assembly may refer to the Genome Reference Consortium's Human Genomic Assembly (Build #38; Assembled: June, 2017), which is accessible on the internet via the U.S. National Center for Biotechnology Information's (NCBI) website.

As used herein, the term “sequencing” or “sequence” as a verb refers to a process whereby the nucleotide sequence of DNA, or order of nucleotides, is determined, such as a nucleotide order AGTCC, etc. The term “sequence” as a noun refers to the actual nucleotide sequence obtained from sequencing; for example, DNA having the sequence AGTCC. Wherein the “sequence” is provided and/or received in digital form, e.g., in a disk or remotely via a server, “sequencing” may refer to a collection of DNA that is propagated, manipulated and/or analyzed using the methods and/or systems of the disclosure.

The term “DNA sequence,” as used herein, generally refers to refers to “raw sequence reads” and/or “consensus sequences.” Raw sequence reads are the output of a DNA sequencer, and typically include redundant sequences of the same parent molecule, for example after amplification. “Consensus sequences” are sequences derived from redundant sequences of a parent molecule intended to represent the sequence of the original parent molecule. Consensus sequences can be produced by voting (wherein each majority nucleotide, e.g., the most commonly observed nucleotide at a given base position, among the sequences is the consensus nucleotide) or other approaches such as comparing to a reference genome. Consensus sequences can be produced by tagging original parent molecules with unique or non-unique molecular tags (e.g., barcode), which allow tracking of the progeny sequences (e.g., after PCR).

The sequencing method can be a first-generation sequencing method, such as Maxam-Gilbert or Sanger sequencing, or a high-throughput sequencing (e.g., next-generation sequencing or NGS) method. A high-throughput sequencing method may sequence simultaneously (or substantially simultaneously) at least 10,000, 100,000, 1 million, 10 million, 100 million, 1 billion, or more polynucleotide molecules. Sequencing methods may include, but are not limited to: pyrosequencing, sequencing-by-synthesis, single-molecule sequencing, nanopore sequencing, semiconductor sequencing, sequencing-by-ligation, sequencing-by-hybridization, Digital Gene Expression (Helicos), massively parallel sequencing, e.g., Helicos, Clonal Single Molecule Array (Solexa/Illumina), sequencing using PACBIO, SOLID, Ion Torrent, or NANOPORE platforms.

The term “whole genome sequencing” refers to a laboratory process that determines the DNA sequence of each DNA strand in a sample. The resulting sequences may be referred to as “raw sequencing data” or “read.” As used herein, a read is a “mappable” read when the sequence has similarity to a region of a reference chromosomal DNA sequence. The term “mappable” may refer to areas that show similarity to and thus “mapped” to a reference sequence, for example, a segment of cfDNA showing similarity to reference sequence in a database, for example, cfDNA having a high percentage of similarity to human chromosomal region 8q248q24.3 in the human genome (HG38) database, is a “mappable read.”

“Deep sequencing” refers to the general concept of aiming for high number of replicate reads of each region of a sequence.

The term “mapping,” as used herein, generally refers to aligning a DNA sequence with a reference sequence based on sequence homology. Alignment can be performed using an alignment algorithm, for example, Needleman-Wunsch algorithm, BLAST, or EMBOSS.

In addition to “WGS,” the genomic compendiums may be obtained using targeted sequencing. In contrast to WGS, the term “targeted sequencing,” as used herein, refers to a laboratory process that determines the DNA sequence of chosen DNA loci or genes in a sample, for example sequencing a chosen group of cancer-related genes or markers (e.g., a target). In this context, the term “target sequence” herein refers to a selected target polynucleotide, e.g., a sequence present in a cfDNA molecule, whose presence, amount, and/or nucleotide sequence, or changes therein, are desired to be determined. Target sequences are interrogated for the presence or absence of a somatic mutation. The target polynucleotide can be a region of gene associated with a disease, e.g., cancer. In some embodiments, the region is an exon.

As used herein, the term “low abundance” in reference to cfDNA refers to an amount of cfDNA in a sample that is less than about 20 ng/mL, e.g., about 15 ng/mL, about 10 ng/mL, or less, e.g., about 9 ng/mL, 8 ng/mL, 7 ng/mL, 6 ng/mL, 5 ng/mL, 4 ng/mL, 3 ng/mL, 2 ng/mL, 1 ng/mL, 0.7 ng/mL, 0.5 ng/mL, 0.3 ng/mL, or less, e.g., 0.1 ng/mL or even 0.05 ng/mL. In some embodiments, the term “low abundance” may be understood in the context of the uniqueness of the marker, e.g., length or base composition. For instance, although a subject's sample may comprise abundant amounts of cfDNA (e.g., >20 ng/mL), the actual number of unique genetic markers (e.g., sSNV, sCNV, indels, SVs) contained in the cfDNA may be very low. Typically, this parameter is expressed as genomic equivalence (GE) or coverage, as described below. In some embodiments, the term “low abundance” may be understood in the context of tumor-specificity of the marker. For example, although a subject's sample may comprise abundant amounts of cfDNA (e.g., >20 ng/mL), a vast majority of the genetic markers (e.g., sSNV, sCNV, indels, SVs) contained in the cfDNA may be redundant and/or associated with the reference (e.g., PBMC gDNA) as well. Typically, this parameter is expressed as tumor fraction (TF), as described below.

As used herein, the terms “tumor-specific” or “tumor-related” in reference to cfDNA refers to differences in DNA sequences of cfDNA in a subject whose cancer formed a tumor, such as a lung cancer patient, when compared to reference DNA, such as when cfDNA is compared to control DNA (gDNA) from a cell that is not a tumor, as described herein. Alternatively, “tumor-specific” may relate to pre-treatment cfDNA when compared to cfDNA collected during or after treatment.

As used herein, the term “read duplicate families” include PCR and sequencing duplicates. Generally, these are independent replicates of the same unique fragment so can be used in statistical test (consensus test) to correct low frequency PCR and sequencing errors.

The term “coverage” or “read depth” relates to the sequencing effort. For instance, coverage of 20× signifies a modest sequencing effort, while a coverage of 35× or more signifies a high sequencing effort and coverage of 5× signifies a low sequencing effort. In embodiments of the present disclosure, the coverage is typically between about 5× to about 100×, particularly between 15× to about 40×, e.g., 20×, 30×, 35×, 40×, 50×, 70×, or more.

As used herein, “depth coverage” refers to the number of unique reads that their mappings overlap at or on specific genomic coordinate.

As used herein, the term “cfDNA coverage mask” refers to mask which represents the genomic territory that is covered by cfDNA reads in a normal cfDNA cohort. As is known in the art, cfDNA coverage is not completely uniform (accessible chromatin genomic regions are less represented), so to eliminate biases a blacklist or a mask may be implemented to permit selective analysis of well-covered regions.

As used herein, the term “read mappability” relates to a numerical value (e.g., percentage identity) or a statistical measure (e.g., confidence estimate) of the accuracy of the mapping of the read with the genome.

As used herein, the term “mutation load” or “N” refers to a level, e.g., number, of an alteration (e.g., one or more genetic alterations, esp., one or more somatic alterations) per a preselected unit (e.g., per mega base pair) in a predetermined genomic window. Mutation load can be measured, e.g., on a whole genome or exome basis, or on the basis of a subset of genome or exome. In certain embodiments, the mutation load measured on the basis of a subset of genome or exome can be extrapolated to determine a whole genome or exome mutation load. In certain embodiments, the mutation load is measured in a sample, e.g., a tumor sample (e.g., a lung tumor sample or a sample acquired or derived from a lung tumor), from a subject, e.g., a subject described herein. Preferably, the mutational load is a measure of the number of mutations per mega base-pairs (1,000,000 bp or MBP) of cfDNA. As is known in the art, the mutation load may vary depending on the type of tumor, genetic lineage, and other subject-specific characteristics such as age, sex, tobacco consumption, etc. In the context of tumor diagnosis, the mutation load may be between about 1000 to about 10000 mutations per MBP, e.g., about 1000, 2000, 4000, 6000, 8000, 10000, 12000, 15000, 20000, 25000, 30000, 40000, 50000, 60000, 70000, 80000, 90000, 10000, or more e.g., about 200000, per MBP. Typically, the mutation load is about 8,000 per MBP in a non-smoker to over 40,000 per MBP in a subject having melanoma.

The term “genomic window,” as used herein, refers to a region of DNA within chosen nucleotide sequence boundaries. Windows may be separate from one another or overlap with one another.

As used herein, the term “tumor fraction” or “TF” relates to a level, e.g., amount, of tumor DNA molecules in relation to normal DNA molecules. In some embodiments, “tumor fraction” refers to the proportion of circulating cell free tumor DNA (ctDNA) relative to the total amount of cell free DNA (cfDNA). Tumor fraction is believed to be indicative of the size of the tumor. Typically, the tumor fraction (TF) is between about 0.001% to about 1%, e.g., about 0.001%, 0.05%, 0.1%, 0.2%, 03%, 0.4%, 0.5%, 0.6%, 0.7%, 0.8%, 0.9%, 1%, or more, e.g., 2%.

The term “abundance” can refer to binary (e.g., absent/present), qualitative (e.g., absent/low/medium/high), or quantitative information (e.g., a value proportional to number, frequency, or concentration) indicating the presence of a particular molecular species. In this context, mutations that are present in higher relative concentrations are associated with a greater number of malignant cells, e.g., with cells that have transformed earlier during the tumorigenic process relative to other malignant cells in the body (Welch et al., Cell, 150: 264-278, 2012). Such mutations, due to their higher relative abundance, are expected to exhibit a higher diagnostic sensitivity for detecting cancer DNA than those with lower relative abundance.

As used herein, “sequencing noise” refers to the noise that is introduced by sequencing instrument, software, or other artefacts during a “run.” There are at least two sources of noise in the sequencing pipeline. First, the DNA mixtures that are produced from input pellets (DNA or cell pellets) are complicated mixtures of cells and therefore any useful signal is diluted by DNA that has no informational content. A second source of noise is due to the specific sequencing technology employed. For example, sequencing noise or “machine” noise can be derived from an ion-to-bases sequencing process, for example with the IONTORENT PGM™ platform. For example, ion detection sequencing that reads bases on pH detection is sensitive to homopolymers and will sometimes read a homopolymer chain as being one base too long or too short.

As used herein, “sequencing error rate” relates to the proportion of sequenced nucleotide being incorrect. For example, in the context of whole genome sequencing, sequencing error rates of about 1 per 1000 bases have been reported in literature (range: error rates are on the order of 0.1-1% per base-call; Wu et al., Bioinformatics, 33(15):2322-2329, 2017).

As used herein, the term “sequencing depth” relates to the number of times the sequenced region is covered by the sequence reads. For example, an average sequencing depth of 10-fold means that each nucleotide within the sequenced region is covered on average by 10 sequence-reads. The chance of detecting a cancer-associated mutation would be expected to increase when the sequencing depth is increased. However, in reality, the odds of detection do not increase linearly with the sequencing depth, as evidenced by the fact that even at a median depth of 42,000×, the fundamental limitation of cfDNA abundance resulted in positive detection of only about 19% of early lung adenocarcinomas (Abbosh et al., Nature, 545(7655):446-451, 2017).

As used herein, the term “noise” in its broadest sense refers to any undesired disturbances (e.g., signal not directly associated with the true event) which may nonetheless be processed or received as true events. Noise is the summation of unwanted or disturbing energy introduced into a system from man-made and natural sources. Noise may distort a signal such that the information carried by the signal becomes degraded or less reliable. The term is contrasted with “signal,” which is a function that conveys information about the behavior or attributes of some phenomenon, e.g., probabilistic association between a marker (SNV, CNV, indel, SV) and a tumor.

As used herein, the term “signal-to-noise ratio” refers the ability to resolve true signal from the noise of a system. Signal-to-noise ratio is computed by taking the ratio of levels of the desired signal to the level of noise present with the signal. Phenomena affecting signal-to-noise ratio include, e.g., detector noise, system noise, and background artifacts. As used herein, the term “detector noise” refers to undesired disturbances (i.e., signal not directly resulting from the intended detected energy) that originate within the detector. Detector noise includes dark current noise and shot noise. Dark current noise in an optical detector system such as a sequencer may result from the various thermal emissions from the photodetector. Shot noise in an optical system is the product of the fundamental particle nature (i.e., Poisson-distributed energy fluctuations) of incident photons as they pass through the photodetector.

The term “filter” is used by those skilled in the art in a number of ways, to mean the discarding or removal of unwanted data, the keeping of wanted data, or both. In the present disclosure, the term “filter” is principally used to imply the keeping of wanted data, e.g., a signal.

The term “base quality” (BQ) score relates to a confidence of the sequencing quality at each nucleobase in a polynucleotide. In some embodiments, the base quality (BQ) includes variable base quality (VBQ) or mean read base quality (MRBQ), both of which are variants of the base quality metric.

The term “mapping-quality” (MQ) score relates to a confidence estimate regarding the accuracy of the mapping of the marker with the genome.

The terms “read position” or “position in read (PIR)” relate to location on a read (e.g., marker) in a nucleotide sequence. As is understood in genomics, many sequencing protocols are prone to various types of amplification induced biases and errors, which may be reduced with the implementation of filters such as “read direction” and “read position” filters. Read direction filter removes variants that are almost exclusively present in either forward or reverse reads. For many sequencing protocols such variants are most likely to be the result of amplification induced errors. Read position filters are implemented to remove systematic errors in a similar fashion as the “read direction filter”, but that is also suitable for hybridization-based data. It removes variants that are located differently in the reads carrying it, than would be expected given the general location of the reads covering the variant site. This is done by categorizing each sequenced nucleotide (or gap) according to the mapping direction of the read and also where in the read the nucleotide is found; each read is divided in parts (e.g., 5 parts) along its length and the part number of the nucleotide is recorded. This gives a total of ten categories for each sequenced nucleotide and a given site will have a distribution between these ten categories for the reads covering the site. If a variant is present in the site, one would expect the variant nucleotides to follow the same distribution. The read position filter carries out a test for measuring significance of the read position, e.g., measuring whether the read position distribution of the variant carrying reads is different from that of the total set of reads covering the site.

As used herein, the term “positional attribute” of a marker (e.g., CNV) relates to a spatial location of the marker in the chromosomal or gene sequence. For instance, the positional attribute of a marker may be measured based on whether it is at least 1000 kilo bases (kb), at least 400 kb, at least 100 kb, at least 20 kb or fewer kb, e.g., 1 kb from a telomere, centromere, or heterochromatin region of a chromosome. CNVs mapped to subtelomeric or pericentromeric regions, which are characterized by chromosomal rearrangement hotspots, may be disfavored. As used herein, the term “representative” in relation to a marker (e.g., CNV) relates to its association with a phenotype or a disease. For instance, previous research has found that CNV calls in immunoglobulin regions are not representative of gDNA and tend to depend substantially on DNA source—e.g., saliva versus blood or lymphoblastoid cell lines versus blood (Need et al., 2009; Wang et al., 2007; Sebat et al., 2004).

As used herein, the term “coverage” or “depth” in DNA sequencing refers to the number of reads that include a given nucleotide in the reconstructed sequence. Coverage histograms are commonly used to depict the range and uniformity of sequencing coverage for an entire data set. They illustrate the overall coverage distribution by displaying the number of reference bases that are covered by mapped sequencing reads at various depths. Mapped “read depth” refers to the total number of bases sequenced and aligned at a given reference base position. Typically, in a sequencing coverage histogram, the read depths are binned and displayed on the x-axis, while the total numbers of reference bases that occupy each read depth bin are displayed on the y-axis. These can also be written as percentages of reference bases.

As used herein “depth coverage” refers to refers to the number of unique reads that their mapping overlap a specific genomic coordinate.

As used herein, the term “read mappability” in relation to CNV refers to the confidence estimate regarding the accuracy of the mapping of the reads related to this CNV with the genome

As used herein, the term “unique read” refers to a read that has a distinctive characteristic, e.g., a unique occurrence in the reference genome. In contrast, a “non-unique read” refers to a read having no or very few distinctive characteristic, e.g., occurring more than once (i.e., repeats) in a read.

As used herein, a genomic “region of interest” or ROI can be any genomic region from which genetic information is desired. The genomic region of interest can comprise a region of a chromosome. The genomic region of interest can comprise a whole chromosome. The chromosome can be a diploid chromosome. In a human genome, for example, the diploid chromosome can be any of chromosomes 1, 2, 3, 4, 5, 6, 7, 8, 9, 10, 11, 12, 13, 14, 15, 16, 17, 18, 19, 20, 21, 22, 23. In some cases, the chromosome can be an X or Y chromosome. In some cases, the genomic region of interest comprises a portion of a chromosome. A genomic region of interest can be of any length. The genomic region of interest can have a length that is between, e.g., about 1 to about 10 bases, about 5 to about 50 bases, about 10 to about 100 bases, about 70 to about 300 bases, about 200 bases to about 1000 bases (1 kb), about 700 bases to about 2000 bases, about 1 kb to about 10 kb, about 5 kb to about 50 kb, about 20 kb to about 100 kb, about 50 kb to about 500 kb, about 100 kb to about 2000 kb (2 Mb), about 1 Mb to about 50 Mb, about 10 Mb to about 100 Mb, about 50 Mb to about 300 Mb. For example, a genomic region of interest can be over 1 base, over 10 bases, over 20 bases, over 50 bases, over 100 bases, over 200 bases, over 400 bases, over 600 bases, over 800 bases, over 1000 bases (1 kb), over 1.5 kb, over 2 kb, over 3 kb, over 4 kb, over 5 kb, over 10 kb, over 20 kb, over 30 kb, over 40 kb, over 50 kb, over 60 kb, over 70 kb, over 80 kb, over 90 kb, over 100 kb, over 200 kb, over 300 kb, over 400 kb, over 500 kb, over 600 kb, over 700 kb, over 800 kb, over 900 kb, over 1000 kb (1 Mb), over 2 Mb, over 3 Mb, over 4 Mb, over 5 Mb, over 6 Mb, over 7 Mb, over 8 Mb, over 9 Mb, over 10 Mb, over 20 Mb, over 30 Mb, over 40 Mb, over 50 Mb, over 60 Mb, over 70 Mb, over 80 Mb, over 90 Mb, over 100 Mb, or over 200 Mb. A genomic region of interest can comprise one or more informative loci. An informative locus can be a polymorphic locus, e.g., comprising two or more alleles. In some cases, the two or more alleles comprise a minor allele.

As used herein, the term “directional” in relation to a read refers to the orientation or manner in which a read is conducted. For instance, in single-end reading, the sequencer reads a fragment from only one end to the other, generating the sequence of base pairs. In paired-end reading it starts at one read, finishes this direction at the specified read length, and then starts another round of reading from the opposite end of the fragment. Paired-end reading improves the ability to identify the relative positions of various reads in the genome, making it much more effective than single-end reading in resolving structural rearrangements such as gene insertions, deletions, or inversions. It can also improve the assembly of repetitive regions. However, and paired-end reads are more expensive and time-consuming to perform than single-end reads.

As used herein, the term “CNV directionality” refers to the direction of change in copy number. For instance, increases in copy number (e.g., augmentations or multiplications) are attributed positively, while reductions (e.g., loss or fragmentation) are attributed negatively.

As used herein, the term “bin” refers to a group of DNA sequences grouped together, such as in a “genomic bin.” In a particular case, the bin may comprise a group of DNA sequences that are binned based on a “genomic bin window,” which includes grouping DNA sequences using genomic windows.

As used herein, the term “estimate” in the context of marker levels is used in a broad sense. As such, the term “estimate” may refer to an actual value (e.g., 1/mbp), a range of values, a statistical value (e.g., mean, median, etc.) or other means of estimation (e.g., probabilistically).

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent.

As used herein, the term “substantially purified” refers to cfDNA molecules that are removed from their natural environment, isolated or separated or extracted, and are at least 60% free, preferably 75% free, more preferably 90% free, and most preferably 99% free from other components with which they are naturally associated.

All publications mentioned herein are incorporated herein by reference for the purpose of describing and disclosing devices, compositions, formulations and methodologies which are described in the publication and which might be used in connection with the present disclosure.

As used herein, the terms “comprise”, “comprises”, “comprising”, “contain”, “contains”, “containing”, “have”, “having” “include”, “includes”, and “including” and their variants are not intended to be limiting, are inclusive or open-ended and do not exclude additional, unrecited additives, components, integers, elements or method steps. For example, a process, method, system, composition, kit, or apparatus that comprises a list of features is not necessarily limited only to those features but may include other features not expressly listed or inherent to such process, method, system, composition, kit, or apparatus.

The practice of the present subject matter may employ, unless otherwise indicated, conventional techniques and descriptions of organic chemistry, molecular biology (including recombinant techniques), cell biology, and biochemistry, which are within the skill of the art.

### Methods

The disclosure relates to methods and systems for detection and/or diagnosis of residual tumors by analyzing markers present in cell-free DNA (cfDNA). The detection can be used alone or in combination with existing technologies to determine the presence or absence of residual tumor, prognosticate the likelihood of having such disease, and also develop therapeutic or prophylactic interventions for such diseases.

In some embodiments, the methods of the disclosure are carried out on a sample obtained from subjects. Preferably, the sample comprises blood (including whole blood), blood plasma, blood serum, hemolysate, lymph, synovial fluid, spinal fluid, urine, cerebrospinal fluid, stool, sputum, mucus, amniotic fluid, lacrimal fluid, cyst fluid, sweat gland secretion, bile, milk, tears, saliva, or earwax. The sample may be treated to remove particular cells using various methods such as such centrifugation, affinity chromatography (e.g., immunoabsorbent means), immuno selection and filtration. Thus, in an example, the sample can comprise a specific cell type or mixture of cell types isolated directly from the subject or purified from a sample obtained from the subject (e.g., purifying T-cells from whole blood). In an example, the biological sample is peripheral blood mononuclear cells (PBMC). In other examples, the sample may be selected from the group consisting of B cells, dendritic cells, granulocytes, innate lymphoid cells (ILCs), megakaryocytes, monocytes/macrophages, natural killer (NK) cells, platelets, red blood cells (RBCs), T cells, thymocytes. In some embodiments, the sample may comprise skin cells, hair follicle cells, sperm, etc.

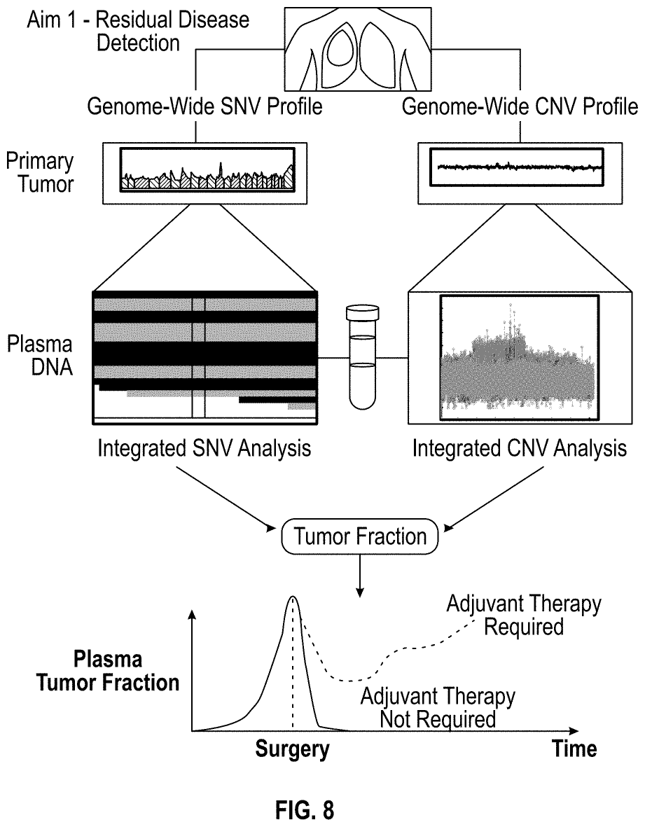

Representative, non-limiting, schematic outlines of the diagnostic methods are provided in FIG. 1 and FIG. 8.

Workflow

FIG. 1A is a flow chart illustrating a method 100 for detection of residual disease, e.g., tumor disease after surgery or post-therapeutic invention (e.g., post-chemotherapy, immunotherapy, targeted therapy, radiation therapy), in accordance with the various embodiments of the present disclosure. Method 100 is illustrative only and embodiments can use variations of method 100. Method 100 can include steps for receiving a compendium of markers; filtering noise associated with the markers based on a number of features; eliminating artefactual noise markers from the compendium to generate subject-specific markers, which are then used to estimate tumor fraction (eTF), which is then used to diagnose the residual disease. It should be noted that TF refers to the fraction of tumor DNA (ctDNA) out of the total plasma DNA (cfDNA). Accordingly, in the present disclosure and elsewhere, the term “ctDNA abundance” may be used interchangeably with the term tumor fraction.

In step 110 of method 100 of FIG. 1A, a compendium of subject-specific genome-wide compendium of reads associated with a plurality of genetic markers (e.g., SNV, CNV, SV, indel) in a biological sample (tumor sample and optionally normal sample) is received from a subject. In some embodiments, the compendium of genetic markers is received in a variant call format (VCF) file. As is understood in the art, VCF files are used in bioinformatics for storing gene sequence variations. The VCF format has been developed with the advent of large-scale genotyping and DNA sequencing projects, such as the 1000 Genomes Project. Alternately, the compendium may be provided in a general feature format (GFF) containing all of the genetic data. Generally, GFF provides features that are redundant because they are shared across the genomes. In contrast, with VCF, only the variations need to be stored along with a reference genome. In some embodiments, the subject's sample is sequenced, e.g., using whole genome sequencing (WGS), and the sequence file is processed, e.g., using a tool such as, for example, genome VCF (gVCF).

In step 120 of method 100 of FIG. 1A, the subject-specific genome wide compendium of genetic markers in a second sample (e.g., plasma or blood) of the subject is detected to generate a representation of tumor-associated genome-wide genetic markers in the patient sample (e.g., plasma or blood sample).

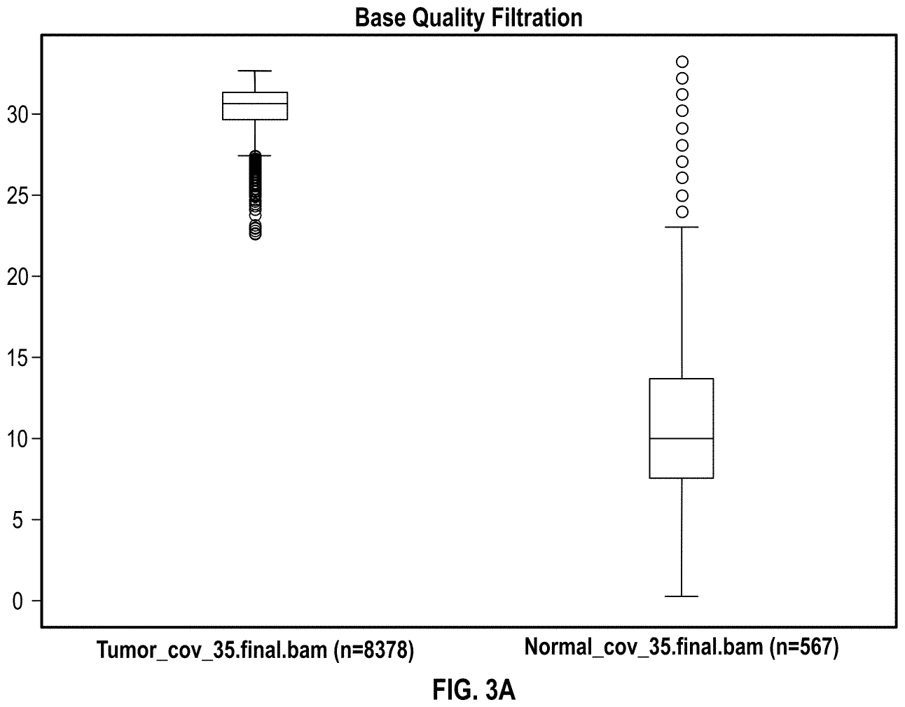

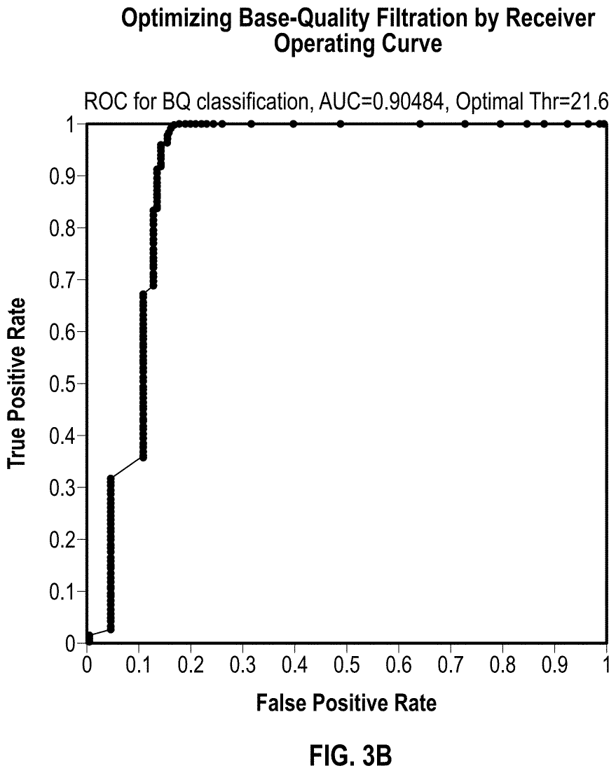

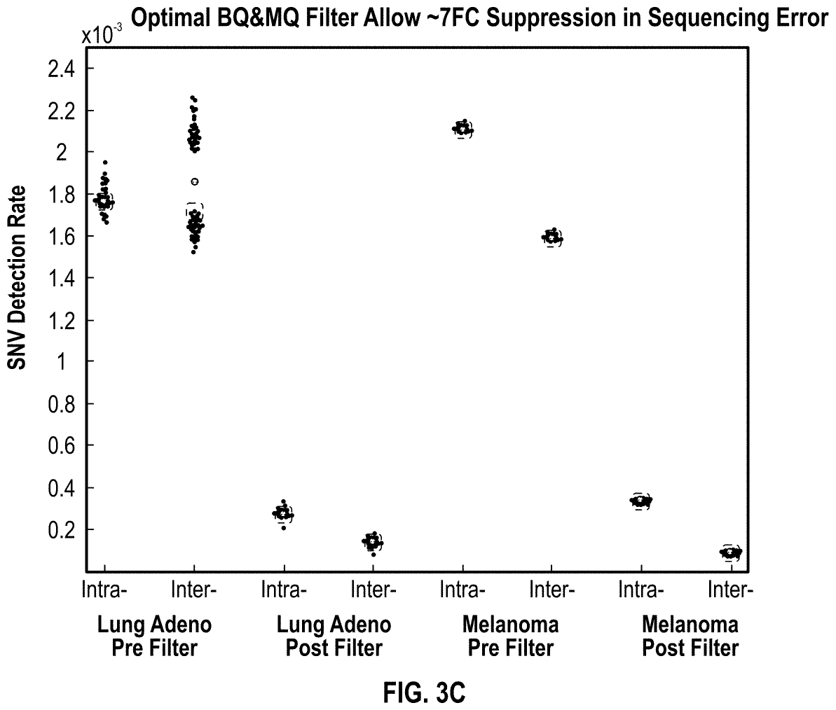

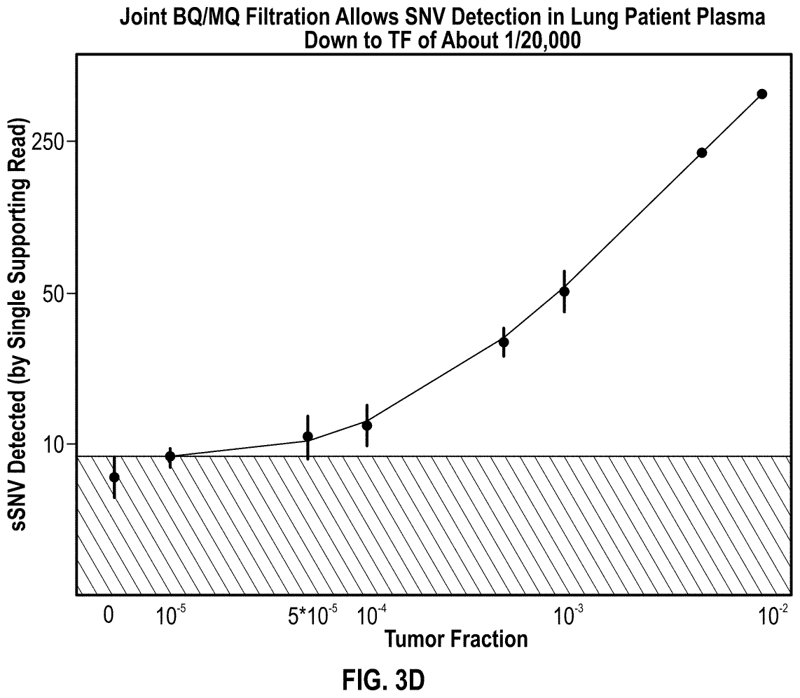

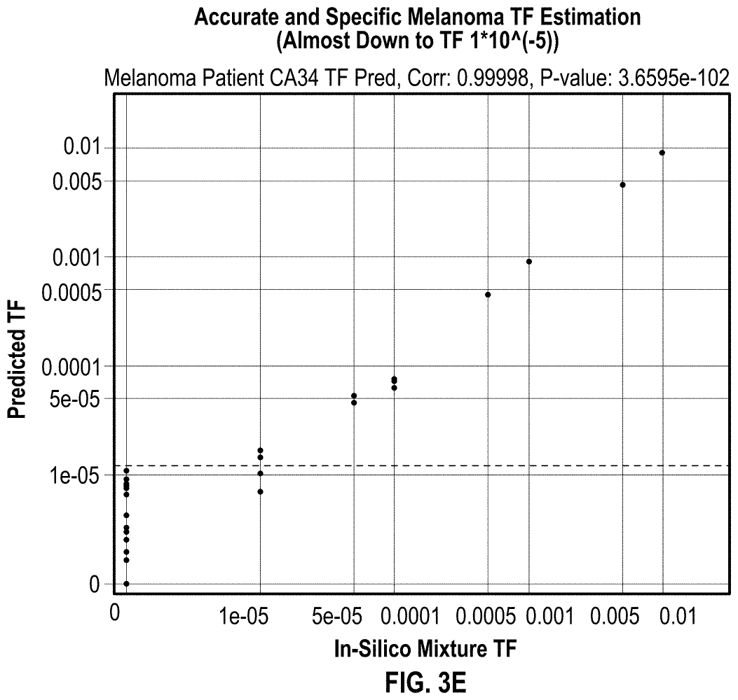

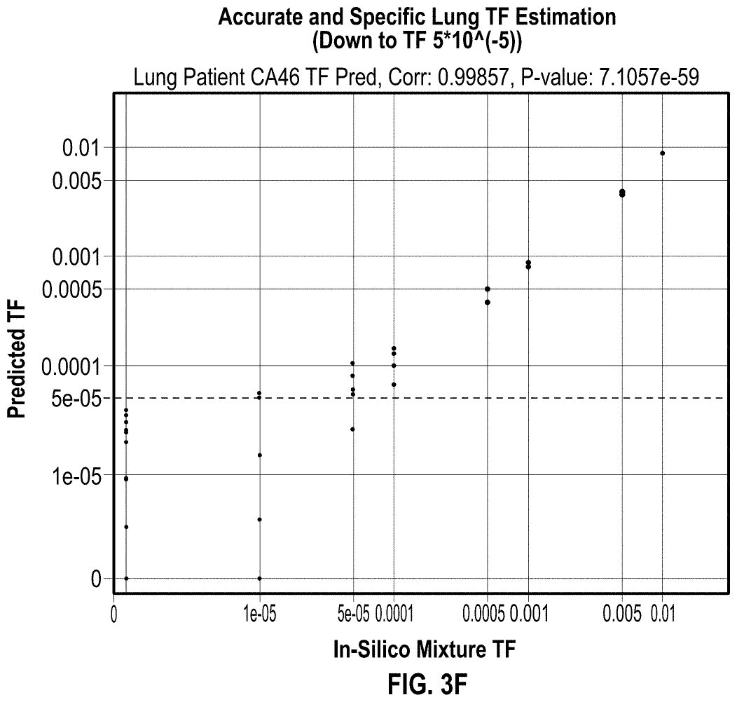

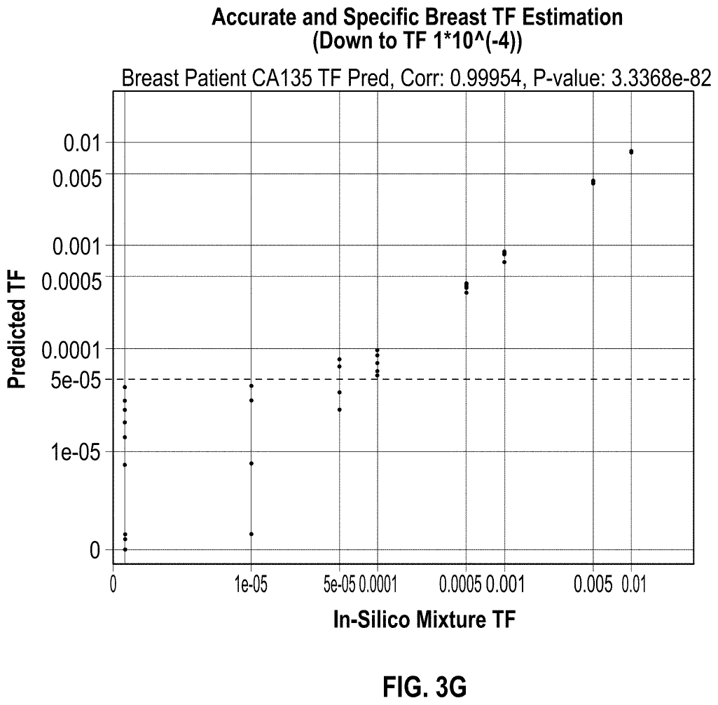

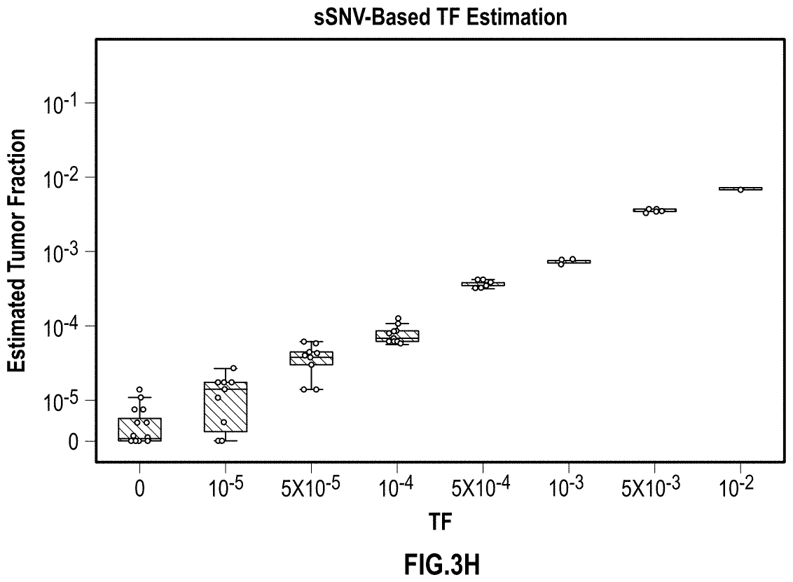

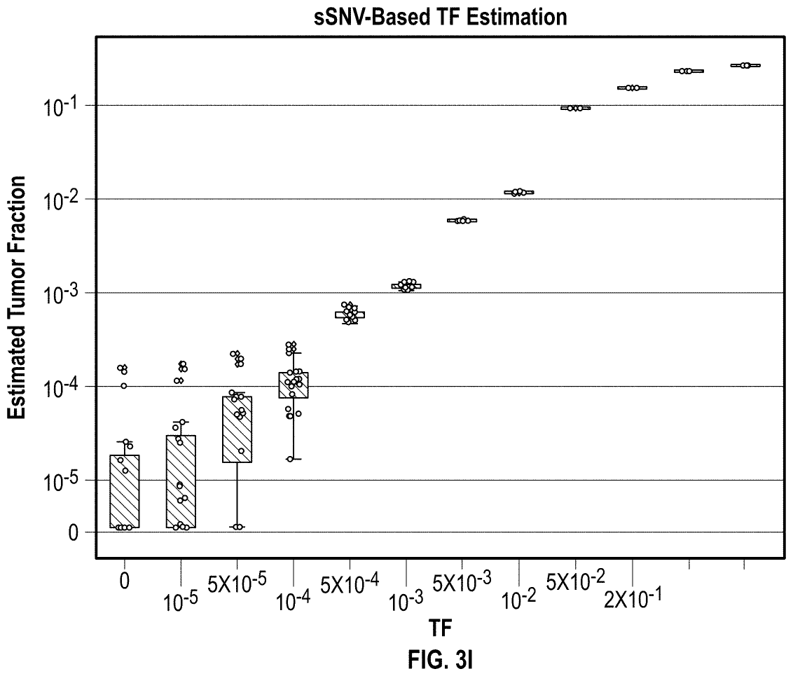

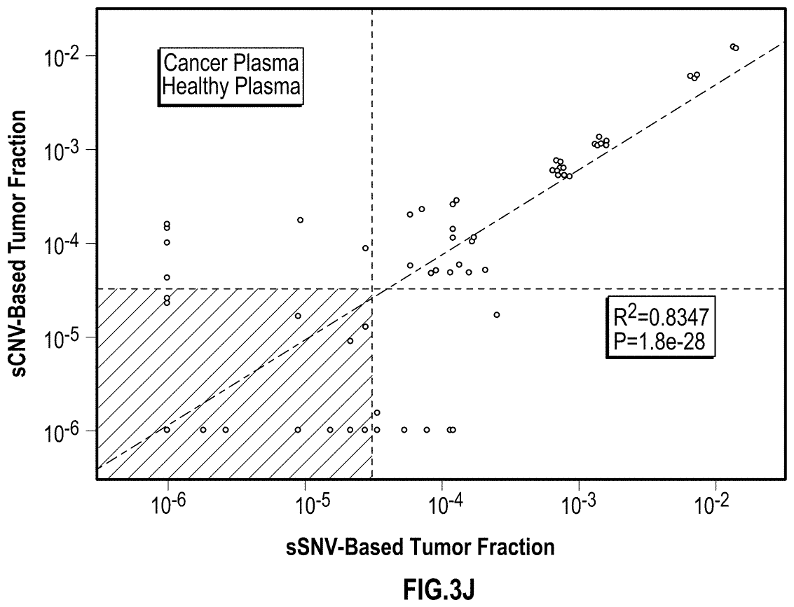

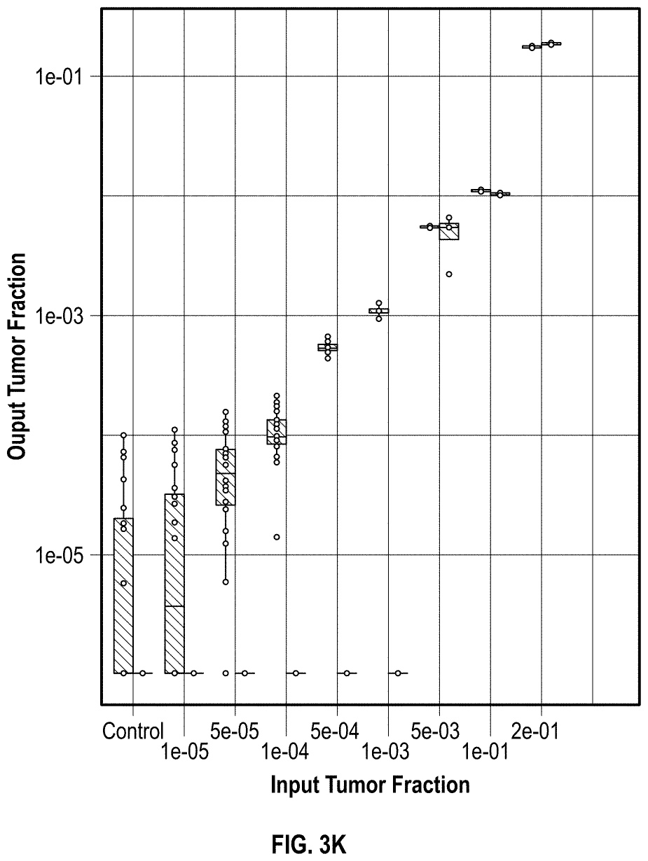

In step 130 of method 100 of FIG. 1A, noise probability (P_{N}) of each marker is analyzed. For instance, wherein the marker is an SNV or indel, the P_{N} may be analyzed as a function of 1) MQ of SNV/indel; 2) fragment length of a read containing SNV/indel; 3) consensus test within read duplicate families that comprises the SNV or Indel, and/or 4) BQ of SNV/indel. Likewise, wherein the marker is a CNV or SV, the probability that the marker is noise-related may be analyzed by statistically classifying each CNV or SV window in the compendium as signal (S) or noise (N) based on: (1) position thereof relative to the centromere, 2) MQ of a read group containing CNV/SV; and/or 3) representation of the CNV window in cfDNA data the artefactual reads. The noise removal step 130 can comprise implementing an optimal receiver operating characteristic (ROC) curve which comprises a probabilistic classification of the genetic markers in the compendium based on a joint base-quality (BQ) and mapping-quality (MQ) score. Typically, the joint BQMQ score is provided as a matrix (x, y), wherein x is the BQ score and y is the MQ score. In exemplary embodiments, a joint BQMQ score between 10 and 50 (for each parameter) is typically employed, e.g., a BQMQ score of (10, 40), (15, 30), (20, 20), (20, 30), (30, 40). In some embodiments, classification of a marker comprises measurement of area under an ROC curve (AUC), which typically represents the probability that a candidate marker, randomly selected among potential markers, shows a value higher than a randomly-extracted control marker. For completely non-informative markers, the ROC curve will approach the rising diagonal (called “chance diagonal” or “chance line”) and AUC will tend to 0.5, i.e., the expected probability for a classification due to chance alone. On the contrary, in the case of a perfect classification the ROC curve will reach the point of the highest theoretical accuracy (sensitivity and specificity both 100%) and AUC will tend to one, i.e., the highest probability value. A representative ROC is provided in FIG. 3B. Pre-filtration error model and post-filtration effects of base quality filter are shown in FIG. 3A. FIG. 3C shows that application of a base quality (BQ) and mapping quality filter (MQ) suppresses sequencing error by about seven fold.

In step 140 of method 100 of FIG. 1A, an estimated tumor fraction (eTF) of the biological sample is computed on the basis of one or more integrative mathematical models. Depending on the marker (e.g., SNV/indels versus CNV/SV), the mathematical model integrates a plurality of process quality metrics, as well as patient-specific attributes, to estimate tumor fractions (TF). Recognizing fundamental differences between SNVs/indels and CNVs/SVs with regard to frequency and also associative properties with a trait (e.g., cancer), the systems and methods of the disclosure involve use of marker-specific mathematical algorithms to estimate tumor fractions. In each case, the mathematical inference model outputs the estimated fraction of tumor DNA in the biological sample (e.g., plasma) based on the number/frequency of the marker, estimated noise, reads, mutation load and/or coverage or depth.

In some embodiments, the methods of the disclosure include estimation of TF based on detection of a plurality of SNV/indel markers. Herein, estimated TF (eTF[SNV]) is computed by integrating process-quality metrics comprising estimated genomic coverage and sequencing noise with patient specific parameters comprising mutation load (N). Preferably, the method comprises computing an estimated tumor fraction (eTF) for SNV/indel markers, wherein eTF[SNV]=1−[1−(M−E(σ)^{R})/N]\hat{ }(1/cov), wherein M is the number of tumor-specific compendium detections in the patient sample, a is a measure of empirically-estimated noise, R is the total number of unique reads in a region of interest (ROI), N is tumor mutation load, and cov is the average number of unique reads per site in the ROI.

In some embodiments, the methods of the disclosure include estimation of TF based on detection of a plurality of CNV/SV markers. Herein, estimated TF (eTF[CNV]) is computed by integrating directional depth of coverage skewed in concordance with tumor CNV/SV directionality wherein amplification of copy number is skewed positively and deletion of copy number is skewed negatively. Preferably, the method comprises computing an estimated tumor fraction (eTF) for CNV markers, wherein eTF[CNV]=(sum_{i}[(P(i)−N(i))*sign[T(i)−N(i)]]−E(sigma))/(sum_{i}[abs(T(i)−N(i))]−E(σ)), wherein P is a median depth value in a genomic window indexed by {i} representing plasma depth coverage, T is a median depth value in a genomic window indexed by {i} representing tumor depth coverage, and N is a median depth value in a genomic window indexed by {i} representing normal depth coverage.

In step 150 of method 100 of FIG. 1A, a residual disease is diagnosed in the subject based on the eTF (computed in step 140) and an empirical threshold calculated by background noise model. In some embodiments, the detection threshold includes empirically measured basal noise TF estimations from healthy samples. In such embodiments, the any eTF that is above a threshold (e.g., at least 2 standard deviations of the noise TF distribution (FPR<2.5%); preferably greater than 3 STD or greater than 5 STD) is defined as positive detection.

As further provided by example workflow 100 illustrated in FIG. 1B, a method is provided for detecting residual disease in a subject in need thereof, in accordance with various embodiments. As provided in step 110 of method 100 in FIG. 1B, the workflow can comprise receiving a first subject-specific genome wide compendium of reads associated with genetic markers from a first biological sample of a subject. The first biological sample can comprising a baseline sample. The first compendium of reads can each comprise reads of a single base pair length. The baseline sample can comprises a tumor sample or a plasma sample. The first biological sample can also include a normal cell sample.

As provided in step 120 of method 100 in FIG. 1B, the workflow can comprise filtering artefactual sites from the first compendium of reads, wherein the filtering comprises removing, from the first compendium of genetic markers, recurring sites generated over a cohort of reference healthy samples. Alternatively or in combination, the filtering can comprise identifying germ line mutations in peripheral blood mononuclear cells of the normal cell sample and removing said germ line mutations from the from the first compendium of genetic markers.

As provided in step 130 of method 100 in FIG. 1B, the workflow can comprise detecting reads from a second subject-specific genome wide compendium of genetic markers in a second biological sample of the subject to generate a tumor-associated genome-wide representation of genetic markers in the second sample.

As provided in step 140 of method 100 in FIG. 1B, the workflow can comprise filtering noise from the first and second genome-wide compendium of reads using at least one error suppression protocol to produce a first filtered read set for the first genome-wide compendium of reads and a second filtered read set for the second genome-wide compendium of reads. The at least one error suppression protocol can comprises calculating the probability that any single nucleotide variation in the first and second compendium is an artefactual mutation, and removing said mutation. The probability can be calculated as a function of features selected from the group consisting of mapping-quality (MQ), variant base-quality (MBQ), position-in-read (PIR), mean read base quality (MRBQ), and combinations thereof. Alternatively, or in combination, the at least one error suppression protocol can include removing artefactual mutations using discordance testing between independent replicates of the same DNA fragment generated from polymerase chain reaction or sequencing processing. Alternatively, or in combination with discordance testing, removing artefactual mutations can include duplication consensus testing wherein artefactual mutations are identified and removed when lacking concordance across a majority of a given duplication family.

As provided in step 150 of method 100 in FIG. 1B, the workflow can comprise computing an estimated tumor fraction (eTF) of the first and second biological sample using the first and second filtered read sets by applying a background noise model to one or more integrative mathematical models.

As provided in step 160 of method 100 in FIG. 1B, the workflow can comprise detecting a residual disease in the subject if the estimated tumor fraction in the second biological sample exceeds an empirical threshold.

As additionally provided by example workflow 100 illustrated in FIG. 1C, a method is provided for detecting residual disease in a subject in need thereof, in accordance with various embodiments. As provided in step 110 of method 100 in FIG. 1C, the workflow can comprise receiving a first subject-specific genome wide compendium of reads associated with genetic markers from a first biological sample of a subject. The first biological sample can comprise a baseline sample. The first compendium of reads can each comprise a copy number variation (CNV). The baseline sample can comprises a tumor sample or a plasma sample.

As provided in step 120 of method 100 in FIG. 1C, the workflow can comprise receiving a second subject-specific genome wide compendium of reads associated with genetic markers from a second biological sample of a subject. The second biological sample can comprise a peripheral blood mononuclear cell sample (PBMC). The second compendium of genetic markers can each comprise a copy number variation (CNV).

As provided in step 130 of method 100 in FIG. 1C, the workflow can comprise filtering artefactual sites from the first and second compendium of reads, wherein the filtering comprises removing, from the first and second compendium of reads, recurring sites generated over a cohort of reference healthy samples. Alternative or in combination, the filtering can comprise identifying shared CNVs between the first and second compendium as germ line mutations and removing said mutations from the first and second compendium of reads.

As provided in step 140 of method 100 in FIG. 1C, the workflow can comprise detecting reads from a third subject-specific genome wide compendium of genetic markers in a third biological sample of the subject to generate a tumor-associated genome-wide representation of genetic markers in the third sample.

As provided in step 150 of method 100 in FIG. 1C, the workflow can comprise normalizing each of the first, second and third compendium of reads to produce a first filtered read set for the first genome-wide compendium of reads, a second filtered read set for the second genome-wide compendium of reads, and a third filtered read set for the third genome-wide compendium of reads.

As provided in step 160 of method 100 in FIG. 1C, the workflow can comprise computing an estimated tumor fraction (eTF) of the third biological samples, using the third filtered read set, by applying a background noise model to one or more integrative mathematical models, the one or more models producing a first eTF using the first filtered read set, and/or the one or more models producing a second eTF using the second filtered read set.

As provided in step 170 of method 100 in FIG. 1C, the workflow can comprise detecting a residual disease in the subject if the estimated tumor fraction in the third biological sample exceeds an empirical threshold.

Schemes

FIG. 1D and FIG. 1E show schematic workflows for practicing the methods of the disclosure. FIG. 1D outlines a workflow that is typically used in cases where the markers of interest comprise SNV/indels; FIG. 1E outlines a workflow that is typically used in cases where the markers of interest comprise CNV/CV. It should be noted that although separate workflows are provided for the purpose of illustration, it is not necessary that they are carried out separately to implement the methods of the disclosure. For example, certain features/elements of the workflows may be utilized in combination to generate an output (e.g., combined estimated tumor fraction based on SNV/indel and CNV/SV) which output is associated with the outcome of interest (e.g., whether the subject with MRD is responding to chemotherapy).

As shown in FIG. 1D, MRD detection based on SNV/indel markers typically utilizes steps for receiving the data; generating patient-specific signatures of SNV/indel; removing/filtering artefactual sites; detection of reads/sites in follow-up samples; suppression of errors using specific algorithms, including, machine learning; correction of reads; detection of sites that provide estimation of tumor fraction; and optionally, orthogonally integrating analysis of secondary features in the genomic data (e.g., analysis of fragment size shifts) to improve sensitivity, specificity and/or reliability of detection.

In the first step of FIG. 1D, genetic data from a baseline sample (typically a tumor sample but also could include pre-treatment plasma, either solely or together with the tumor sample) and a normal sample (typically PBMC but could also include adjacent normal tissue or buccal swab) are received to generate a patient-specific marker signature (e.g., comprising SNVs/indels). Next, a reference list of somatic mutations is called from a baseline sample by filtering artefact sites. Here, germ-line mutations are removed from the sample. Also, somatic mutation calling is performed independently using multiple callers (e.g., MUTECT, STRELKA) using the callers' intersection to generate a list of high confidence mutations. Successively or in parallel, recurrent artefactual sites are generated over a cohort of healthy plasma samples (panel of normal (PON) blacklist or mask), which are removed from the patient detected mutations in order to remove common sequencing or alignment artifacts. The filtered high confidence patient specific dataset of mutations are then used to detect mutations in a follow-up plasma sample. Typically, the follow-up plasma is obtained after surgery, during or after therapy (e.g., chemotherapy), or at follow-up (e.g., checking for recurrence or relapse).

Next, a highly sensitive method that is capable of detecting a single mutated fragment is employed. This step employs one or more error suppression steps. In a first error suppression step, a filtration scheme is used to analyze on a single read basis and quantify the probability for the read to be representing an artefactual mutation. A representative method includes multidimensional classification framework using support vector machine (SVM) classification with a linear kernel. This classification Engine was trained on germline SNP and compared to low variant-allele-fraction (VAF) sequencing artifacts in normal PBMC samples. The classification decision boundary was defined over a multidimensional space including-variant base-quality (VBQ), mapping-quality (MQ), position-in-read (PIR), mean read base quality (MRBQ). To evaluate the classification scheme, validation metrics of the SVM after 10-fold cross validation were compared to Random Forest under the same protocol. SVM classification showed high classification performance, moderately outperforming the random-forest model. The SVM achieved a mean 90.7% sensitivity and 83.9% specificity across all patients (N=10 samples, F1=87.7%, PPV=84.9%).

In a second error suppression step, artefactual mutations generated by PCR or sequencing were corrected using the comparison of independent replicates of the same original DNA fragment. In cfDNA samples, typically paired-end 150 bp sequencing were applied, resulting in overlapping paired reads (overlapping R1 and R2 sequence) given the short size of the typical cfDNA fragment (˜165 bp). Therefore, any discordance between R1 and R2 pairs are regarded as potential sequencing artifacts, which are corrected back to the corresponding reference genome. In addition, recognizing the potential for the creation of independent duplications with any DNA molecule copied multiple times during sequencing and PCR, the duplication families were recognized by 5′ and 3′ similarity as well as alignment position. Each duplication family is then used to check the consensus of a specific mutation across independent replicates, correcting artefactual mutations that do not show concordance in a majority of the duplication family.

Next, the fraction of the patient specific mutations that appear in the plasma is estimated. This parameter obeys a binomial distribution over N independent Bernoulli experiments, where N is the patient mutation load. Each such experiment includes multiple rounds of random samples that depends on the local coverage where the probability of sampling a mutated fragment in each round is the tumor fraction. Therefore, there is a mathematical relationship between the coverage, mutation load, number of detected mutations and the tumor fraction which corresponds to the following equation M=N(1−(1−TF)^{cov})+μ*R, where M denotes the number of mutations detected in the follow-up plasma sample, N denotes the mutation load in the patient-specific mutation pattern, TF denotes the tumor fraction, cov denotes the local coverage in the patient mutation sites, p denotes the noise rate that correspond with the specific patient mutation sites. This relationship allows the calculation of the patient tumor fraction from the mutation detection rate, even in extremely low allele fraction where the mutation allele fraction itself is not informative (mainly represent random sampling between 0 and 1 over the effective coverage-only one supporting read).

In order to address variation in noise between patients with different mutation patterns patient specific mutation signatures are used to calculate the expected noise distribution over a cohort of healthy plasma samples (panel-of-normal, PON). Mainly the same process described above is performed for the detection of the patient specific pattern in healthy samples (PON) or other patients (cross-patient analysis). These detections represent the background noise model for which we calculate the mean and standard-deviation (μ,σ) of artefactual mutation detection rate. Confidence tumor detection and tumor fraction estimation achieved if the patient detected tumor fraction is higher than the artefactual tumor fraction that correspond to 1.5*σ in error rate above the mean.

Next, optionally, the workflow may include orthogonal integrations of calculations based on fragment size shifts. Here, to make the prognostic/diagnostic method more robust, accurate, and/or sensitive, read-based features, e.g., shifts in fragment sizes of DNA, may be orthogonally integrated into the model. The significance of the orthogonal features (in determination of MRD) may be determined using statistical approaches or probabilistic mixture model (e.g., Gaussian model). See Example 3A for detailed overview.

In the exemplified method, high confident tumor-specific detections in the plasma sample are aggregated and converted to an estimation of the fraction of tumor DNA (TF) based on a probabilistic dilution model. The entire detection protocol (detection, error suppression and tumor fraction estimation) is also done on a panel of healthy plasma samples (PON) using the patient specific mutation compendium, calculating the distribution of noisy TF values in healthy samples using the same signature. Following that, tumor detection and estimation is performed only for samples that show tumor fraction that is significantly higher than the PON noisy TF values, using a statistical significance framework (z-score) that insure a low false positive rate (high specificity). An orthogonal confirmation of the presence of tumor DNA in the plasma mutation detections is done using statistical methods (significance tests or GMM) that quantify the intra-patient fragment size shift between the tumor-specific detection list and other random mutation detection list.

Alternately or in combination with the above workflow, the disclosure also relates to detection of residual disease (or monitoring therapy) using CNV/SV markers. As shown in FIG. 1E, MRD detection based on CNV/SV markers typically utilizes steps for receiving the data; generating baseline sample-specific and/or normal sample-specific signatures of CNV/SV; removing germ-line CNV events; filtering artefact windows; detection of window-based median depth coverage in follow-up samples; normalization using, e.g., guanine-cytosine (GC) normalization and/or zscore normalization; detection of tumor CNV signal that provide estimation of tumor fraction; and optionally, orthogonally integrating analysis of secondary features in the genomic data (e.g., analysis of fragment size shifts), so as to improve sensitivity, specificity and/or reliability of detection.

In the first step of FIG. 1E, genetic data from a baseline sample (typically a tumor sample but also could include pre-treatment plasma, either solely or together with the tumor sample) and a normal sample (typically PBMC but could also include adjacent normal tissue or buccal swab) are received to generate a tumor-specific marker signature and also a normal marker signature (e.g., signatures comprising CNVs/SVs). Next, tumor copy-number-variations (T_CNV) are called using the baseline against a panel-of-normal (PON). PBMC copy-number-variations (P_CNV) are called using the PBMC sample against a panel-of-normal (PON). Shared copy-number-variation events are considered as germ-line. Tumor somatic events (sT_CNV, detected only in tumor tissue) and PBMC somatic events (sP_CNV, detected only in PBMC tissue) can be used for tumor fraction detection and estimation.

Next, germ-line variations (e.g., CNV/SV events) are removed from the CNV/SV reference list to generate baseline sCNVs/SVs and/or normal-sCNV/SV. Also, windows with low mappability and/or coverage are filtered. Successively or in parallel, recurrent artefactual sites are generated over a cohort of healthy plasma samples (panel of normal (PON) blacklist or mask), which are removed from windows in order to filter artefactual windows. The filtered high confidence reference CNV/SV segments are used to detect mutations in a follow-up plasma sample. Typically, the follow-up plasma is obtained after surgery, during or after therapy (e.g., chemotherapy), or at follow-up (e.g., checking for recurrence or relapse).

Recurrently artefactual CNV sites are generated over a cohort of healthy plasma samples (panel of normal-PON Blacklist) and are removed from the patient detected mutations in order to remove common sequencing or alignment artifacts such as centromere and repeat regions.

The region-of-interest (ROI) that contains all the genomic segments of the sT_CNV and sP_CNV is then binned to windows (500 bp or more). The depth coverage (read count) in each window is estimated from a follow-up plasma sample (after surgery, during treatment, at follow-up for recurrence). Median depth coverage per window is calculated and divided by the average sample coverage.

Next, depth coverage values are then normalized to correct for GC-content and mappability biases by performing two LOESS regression curve-fitting on the bin-wise GC-fraction and mappability score.

Further batch-effect correction is done using a robust-zscore normalization, which is applied to each sample separately. Briefly, median and median-absolute-deviation (MAD) are calculated based on the neutral regions of each sample and then all CNV bins are normalized by (B(i)−Median)/MAD.

For each bin the depth coverage skew and fragment size center-of-mass (COM) skew is calculated in comparison to a panel of normal (PON) healthy plasma samples. Herein, low tumor fraction samples show a sparse depth coverage skew that is biased by the directionality of the CNV segment-amplification segment will show a bias towards positive depth coverage skew while deletion show a bias towards negative depth coverage skew. On the other end, neutral regions show a random skew without a preferred directionality, so multiplying the differential (plasma −PON) depth coverage skew by the directionality of the CNV segment (amplification multiplied by +1, Deletion multiplied by −1) will sum up the CNV signal across the genome while neutral region noise will be canceled due to random directionality.

This step is done by the following equation [Σᵢ₌₁^{M}(P(i)−N(i))*sign(T(i)−N(i))] where M is the number of windows covering the ROI. P(i) and N(i) are the depth coverage values in window I for the plasma sample and PON, respectively. Sign(T(i)−N(i)) represent the direction of the tumor CNV segment (Amplification multiplied by +1, Deletion multiplied by −1).

Then tumor fraction can be calculated by checking the linear dilution ratio between the cumulative signal detected at the plasma sample in compare to the cumulative signal detected in the tumor. This step is done by the following equation:

$$TF = ( \frac{{\sum }_{i = 1}^{N} [ ( P ( i ) - N ( i ) ) * sign ( T ( i ) - N ( i ) ) ]}{{\sum }_{i = 1}^{N} [ abs ( T ( i ) - N ( i ) ) ]} )$$

wherein N(i), P(i), T(i) represent the patient PBMC, plasma and tumor depth coverage in window I, respectively.

To address variation in noise between patients with different CNV patterns, patient specific CNV signature are used to calculate the expected noise distribution over a cohort of healthy plasma samples (panel-of-normal, PON). Mainly the same process described above in the case of analysis of SNV markers may be performed to detect the patient specific pattern in healthy plasma samples (PON) or other patients (cross-patient analysis). These detections represent the background noise model for which we calculate the mean and standard-deviation (μ,σ) of artefactual mutation detection rate. Confidence tumor detection and tumor fraction estimation achieved if the patient detected tumor fraction is higher than the artefactual tumor fraction that correspond to 1.5*σ in error rate above the mean.

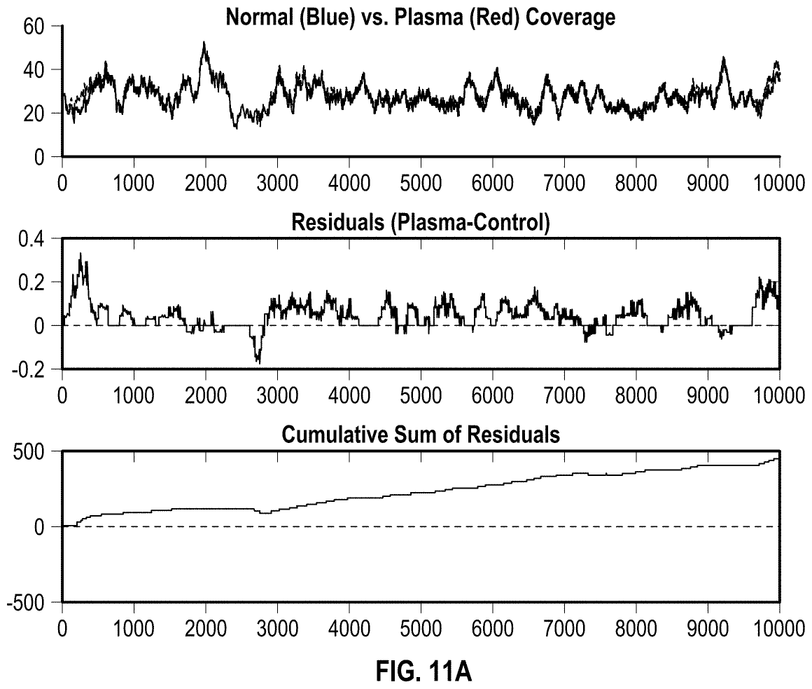

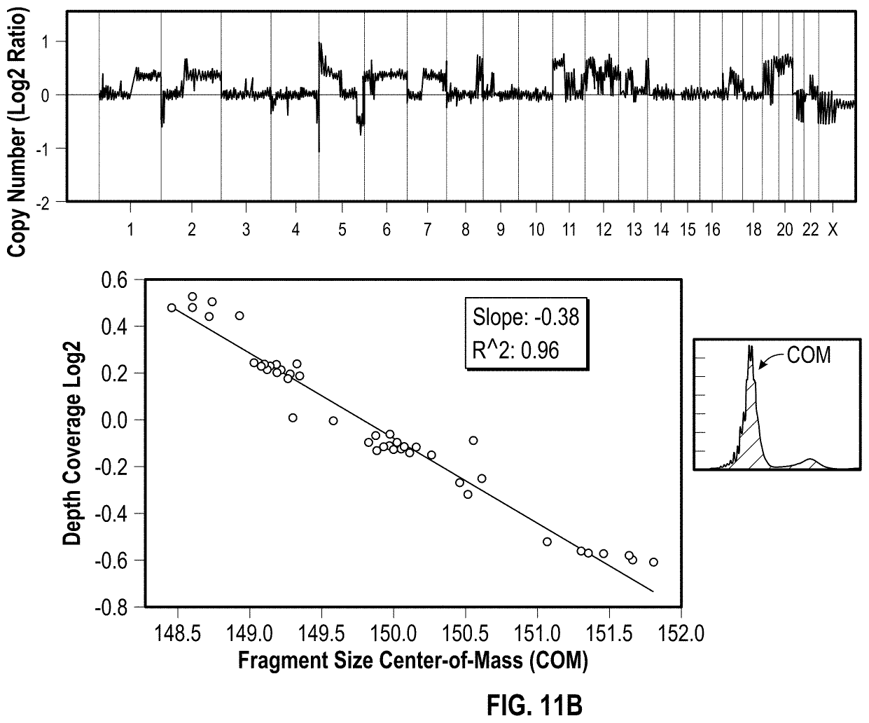

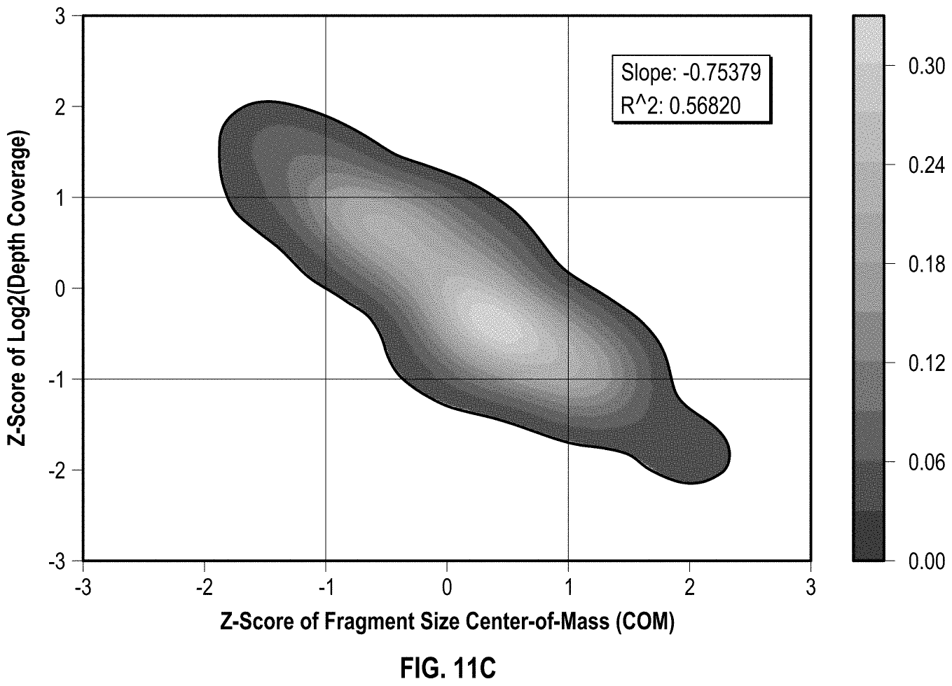

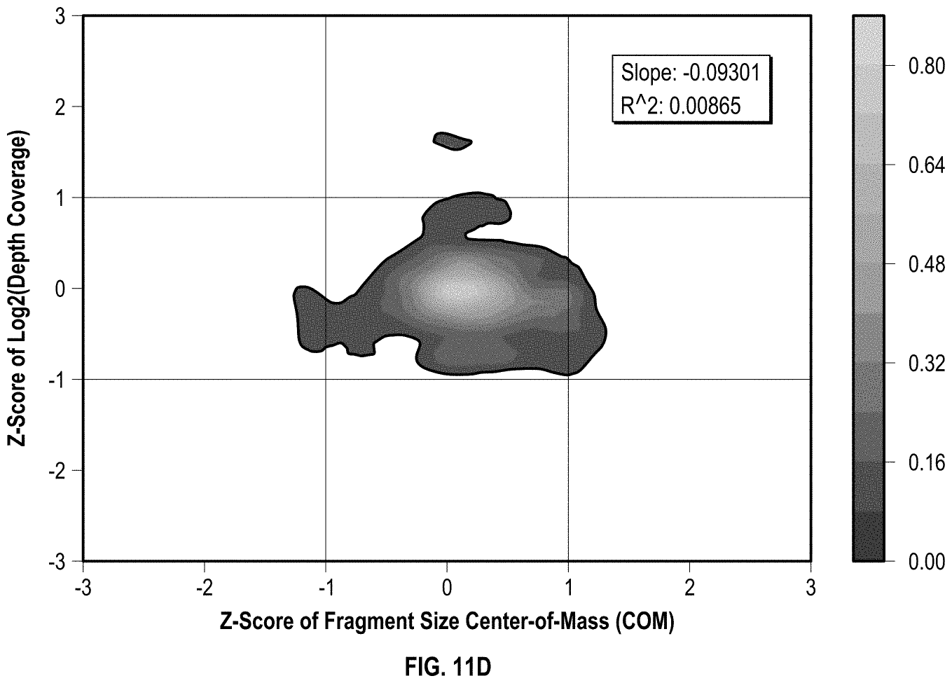

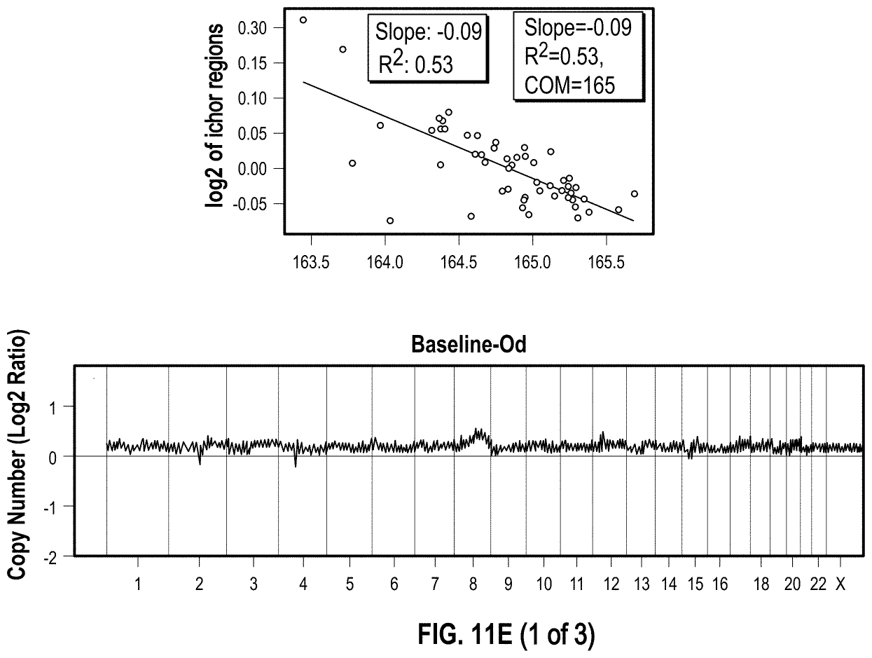

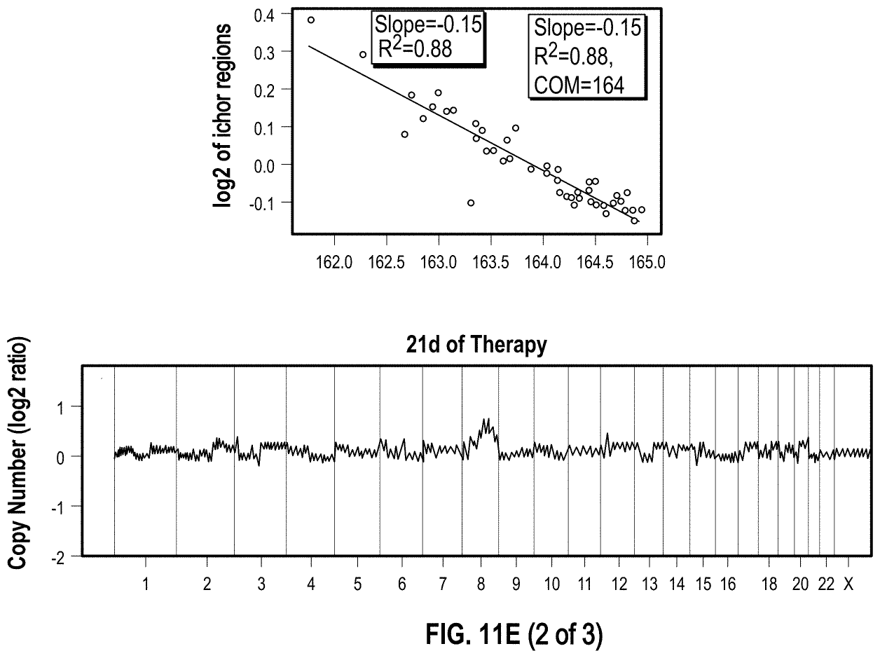

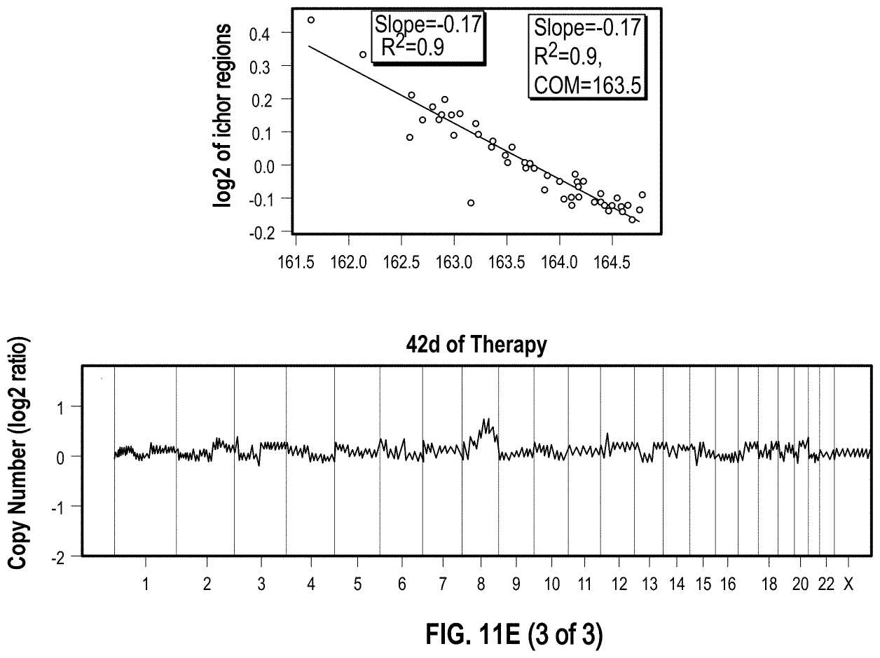

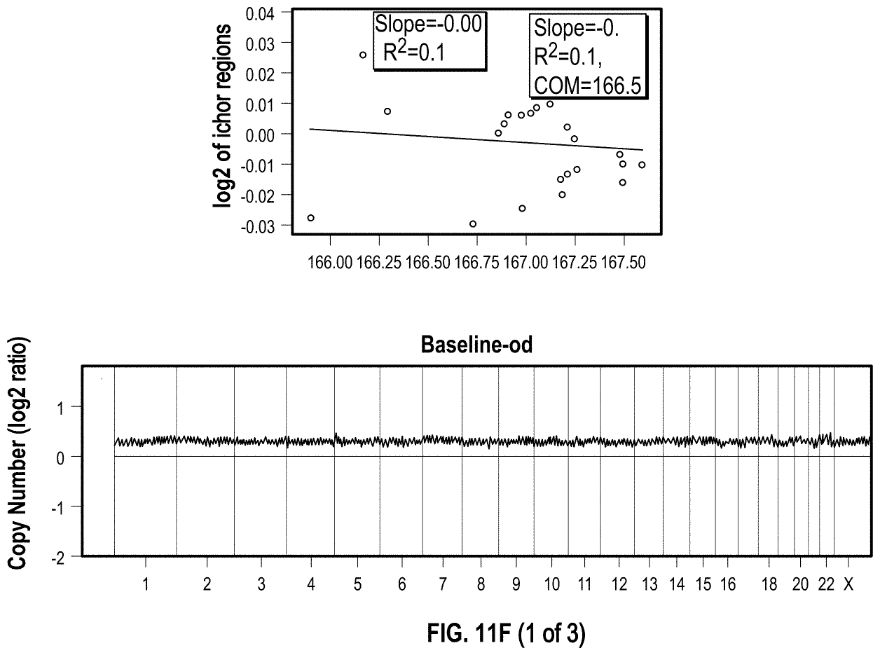

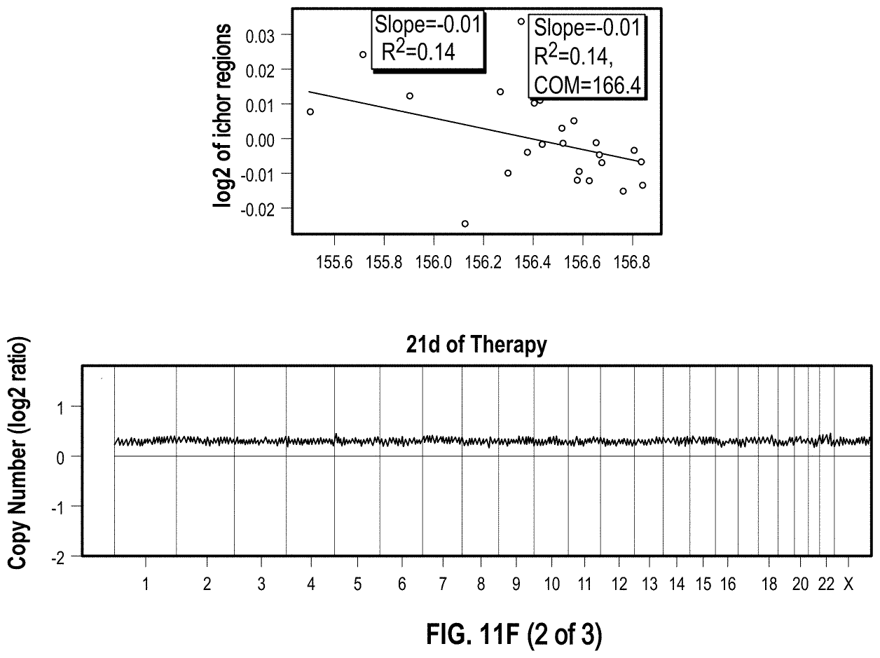

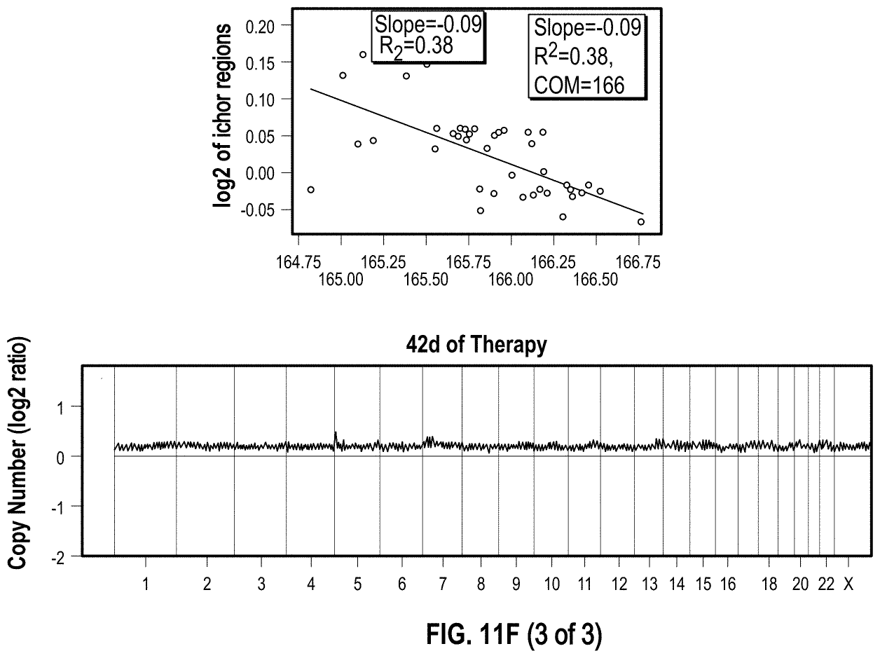

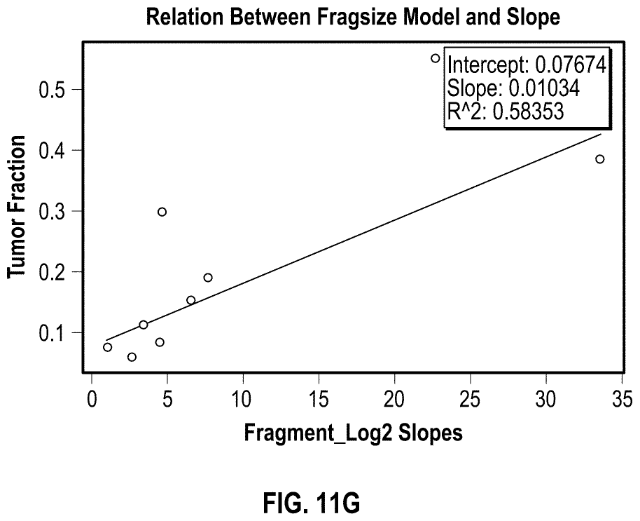

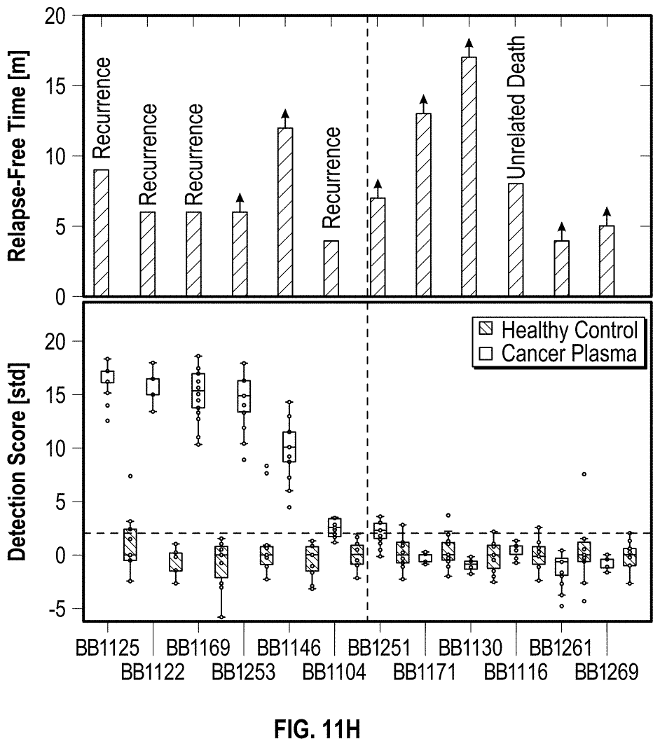

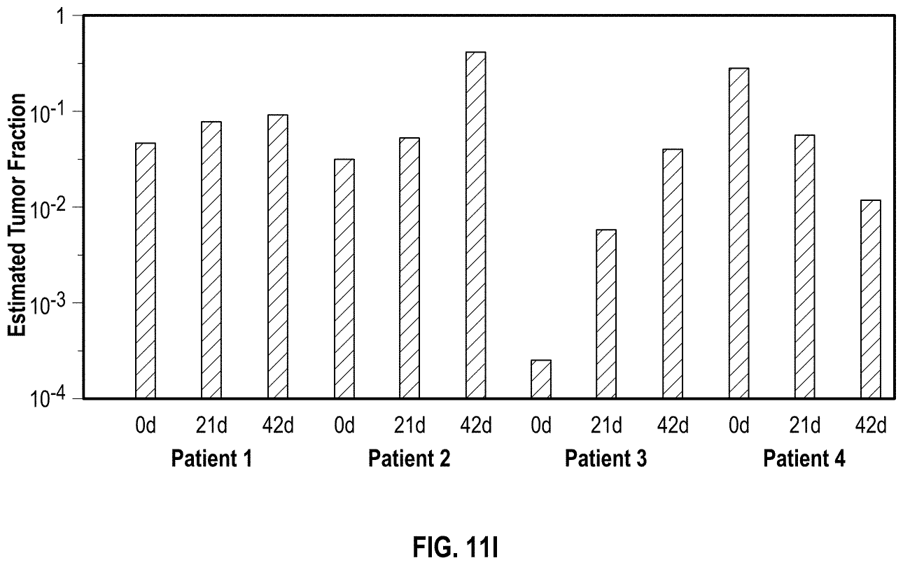

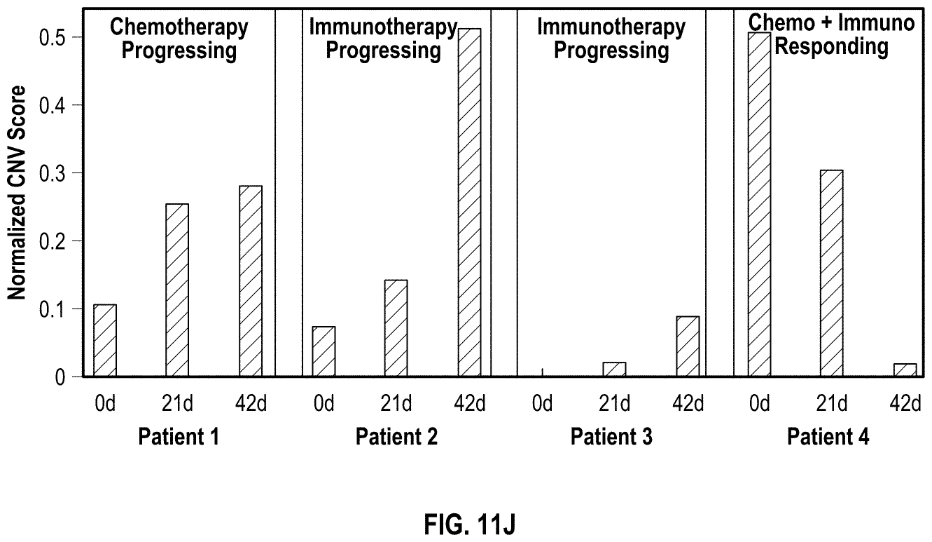

It may also be possible to infer tumor fraction from directional genome-wide depth coverage skew in sP_CNV. Herein, PBMC specific CNV event is expected to decrease its signal due to an increase in tumor DNA fraction (since the tumor DNA do not include this CNV events). Hence, a negative correlation is expected between tumor fraction and sP_CNV detected signal in plasma. Accordingly, multiplying the differential (PBMC-plasma) depth coverage skew by the directionality of the PBMC CNV segment (amplification multiplied by +1, Deletion multiplied by −1) will sum up the PBMC CNV signal across the genome (FIG. 11A).

Then tumor fraction may be calculated by checking the proportion of loss of PBMC CNV signal, e.g., with the equation:

$$TF = ( \frac{{\sum }_{i = 1}^{N} [ ( N ( i ) - P ( i ) ) * sign ( N ( i ) - T ( i ) ) ]}{{\sum }_{i = 1}^{N} [ abs ( N ( i ) - T ( i ) ) ]} )$$

As in the case of MRD estimation using SNV/indel markers, it is possible to orthogonally integrate secondary features into the final calculations. Here, to improve robustness, accuracy, and/or sensitivity/specificity of the detection methods, read-based features, e.g., shifts in fragment sizes of DNA, may be orthogonally integrated into the model. The significance of the orthogonal features (in determination of MRD) may be determined using a generalized linear model (GLM) to orthogonally determine the tumor fraction based on the relationship between CNV depth coverage and fragment size shift. See Example 3B for detailed overview.

It should be appreciated that, with some modifications, the workflows disclosed herein, can also be broadly used for detection of residual disease during or after chemotherapy, immunotherapy, targeted therapy, or a combination thereof, and/or in the course of monitoring the effectiveness of such therapy.

The exemplified method is partly based on the recognition that genome-wide CNV signals in the plasma sample is accumulating only in the case where the coverage skews in the plasma obey the same directionality as the copy-number variations (amplification and deletion) in the baseline tissue (e.g., tumor). Accordingly, tumor DNA ratio can be calculated from the gain-of-signal in the plasma sample from CNV events that are specific to the patient tumor, e.g., using a linear dilution ratio between the cumulative CNV signal in the plasma divided by the cumulative CNV signal in the tumor. Tumor fraction can be orthogonally estimated based on the loss-of-signal from CNV events that are specific only to the patient PBMC (hematopoietic somatic CNV events), with similar mixture dilution model. The entire CNV detection protocol is also done on a panel of healthy plasma samples (PON) using the patient specific copy-number variation compendium, calculating the distribution of noisy TF values in healthy samples using the same CNV signature. Following that, tumor detection and estimation is performed only for samples that show tumor fraction that is significantly higher than the PON noisy TF values, using a statistical significance framework (z-score) that insure a low false positive rate (high specificity). An orthogonal confirmation of the presence of tumor DNA in the plasma is done by checking the relationship (negative correlation) between CNV log 2 values and fragment-size center-of-mass (COM) values across the patient-specific CNV segments, this relationship can then be converted to an orthogonal estimation of the CNV-based TF estimation based on generalized linear model (GLM).

### Machine Learning

Not being bound to a single embodiment and purely for the purpose of illustration, a machine-learning (ML) algorithm was integrated into the existing methodology at an individual, or combination of individual steps, in accordance with various embodiments herein. ML can be incorporated to optimize the results coming out of the algorithm (e.g., neural network, ML algorithm, etc.), by utilization of inputted training data sets, cross reference of output to known answers, backpropagation, and adjustment of weighting factors and parameters associated with the given ML algorithm in a repeating loop to arrive at a threshold quality of data output. In subsequent steps, the prediction power of the model on the test dataset may be validated, e.g., using a probability model such as logistic regression (e.g., optimized or trained in conjunction or in the alternative). Optionally, a resampling may be performed to obtain an unbiased appraisal of the model's likely future performance. Features of ROC curve, such as, area-under-the curve (also called c-index) or concordance probability from a statistical test such as the Wilcoxon-Mann-Whitney test, may provide a good summary measure of pure predictive discrimination.

Preferably, the ML algorithm adaptively and/or systemically filters sequencing noise associated with each read in the compendium on the basis of one or more quality filters or read features. In some embodiments, the ML algorithm implements base quality (BQ) filters (more specifically, variable base quality (VBQ) or mean read base quality (MRBQ)) for filtering noise. In some embodiments, the ML algorithm implements mapping quality (MQ) filters for filtering noise. In some embodiments, the ML algorithm implements position in read (PIR) filters for filtering noise. In some embodiments, the ML algorithm implements a combination of filters.

In some embodiments, the machine learning (ML) method used in the systems and/or methods of the disclosure comprises deep convolutional neural network (CNN), recurrent neural network (RNN), random forest (RF), support vector machine (SVM), discriminant analysis, nearest neighbor analysis (KNN), ensemble classifier, or a combination thereof, preferably, support vector machine (SVM). In some embodiments, the ML has been trained to distinguish between cancer altered sequencing reads and reads altered by sequencing or PCR errors. In some embodiments, the ML has been trained on a large whole-genome sequenced (WGS) cancer dataset comprising billions of reads across tumor mutations and normal sequencing errors. In some embodiments, the ML is capable of (a) identifying, with high precision, sequencing or PCR artifacts and (b) integrating sequence context and read specific features.

The disclosure further relates to systems and programs that utilize ML, e.g., Engine, to adaptively and/or systemically filter sequencing noise. The disclosure also relates to computer-readable storage medium containing a program for detecting tumor markers comprising somatic mutations in a genomic read, the program utilizing ML, e.g., a support vector machine (SVM).

As is known in the art, a convolutional neural network (CNN) generally accomplishes an advanced form of processing and classification/detection by first looking for low level features such as, for example, repeat sequences in a read, and then advancing to more abstract (e.g., unique to the type of reads being classified) concepts through a series of convolutional layers. A CNN can do this by passing data through a series of convolutional, nonlinear, pooling (or downsampling, discussed below), and fully connected layers, and get an output. Again, the output can be a single class or a probability of classes that best describes the data or detects objects on the data.

Regarding layers in a CNN, the first layer is generally a convolutional layer (conv). This first layer will process the read's representative array using a series of parameters. Rather than processing the data as a whole, a CNN will analyze a collection of data sub-sets using a filter (or neuron or kernel). The sub-sets will include a focal point in the array as well as surrounding points. For example, a filter can examine a series of 5×5 areas (or regions) in a 32×32 representation. These regions can be referred to as receptive fields. Since the filter generally will possess the same depth as the input, a representation with dimensions of 32×32×3 would have a filter of the same depth (e.g., 5×5×3). The actual step of convolving, using the exemplary dimensions above, would involve sliding the filter along the input data, multiplying filter values with the original representation values of the data to compute element wise multiplications, and summing these values to arrive at a single number for that examined region of the representation.

After completion of this convolving step, using a 5×5×3 filter, an activation map (or filter map) having dimensions of 28×28×1 will result. For each additional layer used, spatial dimensions are better preserved such that using two filters will result in an activation map of 28×28×2. Each filter will generally have a unique feature it represents that, together, represent the feature identifiers required for the final data output. These filters, when used in combination, allow the CNN to process data input to detect those features present at each representation. Therefore, if a filter serves as a curve detector, the convolving of the filter along the data input will produce an array of numbers in the activation map that correspond to high likelihood of a curve (high summed element wise multiplications), low likelihood of a curve (low summed element wise multiplications) or a zero value where the input volume at certain points provided nothing that would activate the curve detector filter. As such, the greater number of filters (also referred to as channels) in the Cony, the more depth (or data) that is provided on the activation map, and therefore more information about the input that will lead to a more accurate output.

Balanced with accuracy of the CNN is the processing time and power needed to produce a result. In other words, the more filters (or channels) used, the more time and processing power needed to execute the Conv. Therefore, the choice and number of filters (or channels) to meet the needs of the CNN method should be specifically chosen to produce as accurate an output as possible while considering the time and power available.

To further enable a CNN to detect more complex features, additional Convs can be added to analyze what outputs from the previous Conv (e.g., activation maps). For example, if a first Conv looks for a basic feature such as a curve or an edge, a second Conv can look for a more complex feature such as shapes, which can be a combination of individual features detected in an earlier Conv layer. By providing a series of Convs, the CNN can detect increasingly higher level features to eventually arrive at a probability of detecting the specific desired object. Moreover, as the Convs stack on top of each other, analyzing the previous activation map output, each Conv in the stack is naturally going to analyze a larger and larger receptive field by virtue of the scaling down that occurs at each Conv level, thereby allowing the CNN to respond to a growing region of representation space in detecting the object of interest.

A CNN architecture generally consists of a group of processing blocks, including at least one processing block for convoluting an input volume (data) and at least one for deconvolution (or transpose convolution). Additionally, the processing blocks can include at least one pooling block and unpooling block. Pooling blocks can be used to scale down data in resolution to produce an output available for Conv. This can provide computational efficiency (efficient time and power), which can in turn improve actual performance of the CNN. Those these pooling, or subsampling, blocks keep filters small and computational requirements reasonable, these blocks can coarsen the output (can result in lost spatial information within a receptive field), reducing it from the size of the input by a specific factor.

Unpooling blocks can be used to reconstruct these coarse outputs to produce an output volume with the same dimensions as the input volume. An unpooling block can be considered a reverse operation of a convoluting block to return an activation output to the original input volume dimension. However, the unpooling process generally just simply enlarges the coarse outputs into a sparse activation map. To avoid this result, the deconvolution block densifies this sparse activation map to produce both and enlarged and dense activation map that eventually, after any further necessary processing, a final output volume with size and density much closer to the input volume. As a reverse operation of the convolution block, rather than reducing multiple array points in the receptive field to a single number, the deconvolution block associate a single activation output point with a multiple outputs to enlarge and densify the resulting activation output.

It should be noted that while pooling blocks can be used to scale down data and unpooling blocks can be used to enlarge these scaled down activation maps, convolution and deconvolution blocks can be structured to both convolve/deconvolve and scale down/enlarge without the need for separate pooling and unpooling blocks.

The pooling and unpooling process can have drawbacks depending on the objects of interest being detected in data input. Since pooling generally scales down data by looking at sub-data windows without overlap of windows, there is a clear loss of spatial info as scale down occurs.

A processing block can include other layers that are packaged with a convolutional or deconvolutional layer. These can include, for example, a rectified linear unit layer (ReLU) or exponential linear unit layer (ELU), which are activation functions that examine the output from a Conv in its processing block. The ReLU or ELU layer acts as a gating function to advance only those values corresponding to positive detection of the feature of interest unique to the Conv.

Given a basic architecture, the CNN is then prepared for a training process to hone its accuracy in data classification/detection (of objects of interest). This involves a process called backpropagation (backprop), which uses training data sets, or sample data used to train the CNN so that it updates its parameters in reaching an optimal, or threshold, accuracy. Backpropagation involves a series of repeated steps (training iterations) that, depending on the parameters of the backprop, will either slowly or quickly train the CNN. Backprop steps generally include a forward pass, loss function, backward pass, and parameter (weight) update according to a given learning rate. The forward pass involves passing a training data through the CNN. The loss function is a measure of error in the output. The backward pass determines the contributing factors to the loss function. The weight update involves updating the parameters of the filters to move the CNN towards optimal. The learning rate determines the extent of weight update per iteration to arrive at optimal. If the learning rate is too low, the training may take too long and involve too much processing capacity. If the learning rate is too fast, each weight update may be too large to allow for precise achievement of a given optimum or threshold.

The backprop process can cause complications in training, thus leading to the need for lower learning rates and more specific and carefully determined initial parameters upon start of training. One such complication is that, as weight updates occur at the conclusion of each iteration, the changes to the parameters of the Convs amplify the deeper the network goes. For example, if a CNN has a plurality of Convs that, as discussed above, allows for higher level feature analysis, the parameter update to the first Conv is multiplied at each subsequent Conv. The net effect is that the smallest changes to parameters can have large impact depending on the depth of a given CNN. This phenomenon is referred to as internal covariate shift.

In general, the CNN of the disclosure can adaptively and/or systemically filter sequencing noise. In some embodiments, the CNN architecture was designed based on the inventors' recognition that tri-nucleotide contexts contain distinct signatures involved in mutagenesis. Accordingly, the CNN convolves over all features (columns) at a position using a perceptive field of size three. After two successive convolutional layers, down sampling is applied by maxpooling with a receptive field of two and a stride of two, forcing the model in the Engine to retain only the most important features in small spatial areas. The resulting architecture maintains spatial invariance when convolving over trinucleotide windows and captures a “quality map” by collapsing the read fragment into 25 segments, each representing approximately an eight-nucleotide region. The final classification is made by applying the output of the last convolutional layer directly to a sigmoid fully-connected layer. The CNN employs a simple logistic regression layer instead of a multi-layer perceptron or global average pooling in order to retain the features associated with position in the genomic read.

To train Engine, a variety of lung cancer patients and their matching systemic error profiles are first sampled. The goal of the training exercise is to use a training scheme that allows detection of true somatic mutations with high sensitivity and also reject candidate mutations caused by systemic errors. A mixture of samples, e.g., a complete tumor sample and a healthy tissue sample from a subject, e.g., who has or is suspected of having cancer, may be used in the training.

### Upstream Steps:

### Receiving Genetic Data

In some embodiments, genetic data is received in situ from a subject's biological sample (e.g., tumor sample or a normal cell sample comprising PBMC). This is primarily accomplished by sequencing. In some embodiments, the sample may be purified using conventional methods to obtain sub-populations of cells. For example, PBMC can be purified from whole blood using various known Ficoll based centrifugation methods (e.g., Ficoll-Hypaque density gradient centrifugation). Other cells such as T-cells can also be purified by selecting for the appropriate phenotype using techniques such as immunomagnetic cell sorting (e.g., DYNABEADS, Invitrogen, Carlsbad, CA, USA). For example, T-cells can be purified using a two-step selection process that firstly removes CD8+ cells and then selects CD4+ cells. Cell population purity can be confirmed by assessing the appropriate markers such as CD19-FITC, CD3-PE, CD8-PerCP, CD11c-PE Cy7, CD4-APC and CD14-APC Cy7 using commercially available antibodies (e.g., BD Biosciences).

After sample preparation, DNA is extracted from the sample for marker analysis. In an example, the DNA is genomic DNA. Various methods of isolating DNA, in particular genomic DNA are known to those of skill in the art. In general, known methods involve disruption and lysis of the starting material followed by the removal of proteins and other contaminants and finally recovery of the DNA. For example, techniques involving alcohol precipitation; organic phenol/chloroform extraction and salting out have been used for many years to extract and isolate DNA. One example of DNA isolation is exemplified below (e.g. Qiagen ALL-PREP kit). However, there are various other commercially available kits for genomic DNA extraction (Thermo-Fisher, Waltham, MA; Sigma-Aldrich, St. Louis, MO). Purity and concentration of DNA can be assessed by various methods, for example, spectrophotometry.

In some embodiments, the genetic data comprises a compendium of genetic markers, which are compiled in a variant call format (VCF) file. As is understood in the art, VCF files are used in bioinformatics for storing gene sequence variations. The VCF format has been developed with the advent of large-scale genotyping and DNA sequencing projects, such as the 1000 Genomes Project. Alternately, the compendium may be provided in a general feature format (GFF) containing all of the genetic data. Generally, GFF provides features that are redundant because they are shared across the genomes. In contrast, with VCF, only the variations need to be stored along with a reference genome.

Microarray technologies are widely used in the detection of markers of the disclosure, such as SNVs/indels and CNVs/SVs. For instance, array comparative genomic hybridization (array CGH) and single nucleotide polymorphisms (SNP) microarrays may be used. In traditional array CGH, reference and test DNAs are fluorescence-labeled and hybridized to arrays, and the signal ratio is used as an estimate of the copy number (CN) ratio. SNP microarrays are also based on hybridization, but a single sample is processed on each microarray, and intensity ratios are formed by comparing the intensity of the sample under investigation to a collection of reference samples or to all other samples that are studied. While microarray/genotyping arrays are efficient for large CNV detection, they are less sensitive for detecting CNVs of short genes or DNA sequences (e.g., with a length of less than about 50 kilobases (kb)).

In some embodiments, markers of the disclosure may be detected using next generation sequencing (NGS). By providing a base-by-base view of the genome, NGS allows detection of small or novel CNVs that may remain undetected by arrays. Examples of suitable NGS methods may include whole-genome (WGS), whole-exome sequencing (WES), or targeted exome sequencing (TES). Preferably, the sequencing method employs WGS.

In some embodiments, a subject's sample is sequenced, e.g., using whole genome sequencing (WGS), and called (for SNV/indel and/or CNV/CV markers) using standard methods. For example, SNV calling from NGS data utilizes computational methods for identifying the existence of single nucleotide variants (SNVs) from the results of next generation sequencing (NGS) experiments. Due to the increasing abundance of NGS data, these techniques are becoming increasingly popular for performing SNP genotyping, with a wide variety of algorithms designed for specific experimental designs and applications. Similarly, several bioinformatics approaches to detect CNVs from next-generation sequencing data (Pirooznia et al., Front Genet., 6: 138, 2015). In some embodiments, the sample is processed and sequenced to obtain a sequence file, and the sequence file is processed, e.g., using a tool such as, for example, genome VCF or exome VCF (eVCF).

In some embodiments, the methods of the disclosure may involve generating a compendium of genetic markers. A typical compendium comprises genetic data of whole genome sequenced tumor sample as well as a control (e.g., PMBC). The tumor sample preferably includes resected tumors or FNA, e.g., adenocarcinoma of the lung or melanoma of the skin. The control sample comprises preferably comprises PMBCs that are obtained using Ficoll separation, as provided above. Admixtures are then created and markers therein are analyzed using the computational methods of the disclosure.

In some embodiments, the methods of the disclosure may include classifying the genetic data into distinct components on the basis of markers contained therein, e.g., SNVs, CNVs, indels, SVs, mutations, deletions, fusions, etc. In preferred embodiments, the classification step may include separate binning of somatic SNVs (sSNV) and somatic CNVs (sCNV) markers which are noise-filtered and analyzed separately on the basis of computational methods of the disclosure. Herein, computational methods for analyzing SNV markers for noise and uniqueness may differ from the methods for analyzing CNVs. In some embodiments, the computational analysis of SNVs or indels may be performed sequentially with the computational analysis of CNVs or SVs. In some embodiments, the analyses may be performed together.

The present disclosure provides the use of mathematical algorithms and computational methods to (a) filter artefactual noise; and (b) to screen true markers.

With regard to noise cancellation, wherein the marker is an SNV or indel, artefactual noise is cancelled on the basis of a plurality of parameters comprising base quality and/or mapping quality. Typically, the BQ score is a measure of the quality of the identification of the nucleobases generated by automated DNA sequencing. It may be determined using routine methods, e.g., Phred quality scores, which are assigned to each nucleotide base call in automated sequencer traces. Phred quality scores (Q) are defined as a property which is logarithmically related to the base-calling error probabilities (P). For example, if Phred assigns a quality score of 30 to a base, the chances that this base is called incorrectly are 1 in 1000. Typically, the BQ of a sequencing read is between 10 and 50, e.g., a BQ score of 10, 15, 20, 25, 30 35 or 40.

Also in the context of sSNV or indel markers, the mapping quality (MQ) score is a measure of the confidence that a read actually comes from the position it is aligned to by the mapping algorithm. It may be determined using routine methods, e.g., mapping quality scores (see, Li et al., Genome Research 18:1851-8, 2008). Typically, the MQ of a read is between 10 and 50, e.g., a MQ score of about 10, 15, 20, 25, 30, 35, or 40.

In some embodiments, noise is eliminated by implementing an optimal receiver operating characteristic (ROC) curve which comprises a probabilistic classification of the genetic markers in the compendium based on a joint base-quality (BQ) and mapping-quality (MQ) score. Typically, the joint BQMQ score is provided as a matrix (x, y), wherein x is the BQ score and y is the MQ score. In exemplary embodiments, a joint BQMQ score between 10 and 50 (for each parameter) is typically employed, e.g., BQMQ score of (10, 40), (15, 30), (20, 20), (20, 30), etc.

Although not bound by any specific theory, in some embodiments, the elimination step filters “noise” markers having low base quality and/or mapping quality from the compendium of markers that are initially identified to be strongly associated with a disease. In some embodiments, the elimination step may comprise taking each marker that meets the threshold probability of detection (P_{D}), classifying said marker as signal or noise based on an ROC curve of the marker; and eliminating the marker from the compendium if it is classified as noise. Alternately, a scoring system comprising, for example, a ratio of probability of detection (P_{D}) to probability of noise (P_{N}) may be used to eliminate markers that do not meet a threshold score.

In addition to BQ and MQ, the read position (RP) may also affect the quality of the signal. In the context of sSNV or indel markers, RP may be mapped, for example, by mapping the position of the initial base of the sequencing read. Other factors that influence marker quality include, e.g., specific sequence contexts that are associated with higher probability of sequencing errors (Chen et al., Science, 355(6326):752-756, 2017). In this regard, true mutations are frequently mappable to their own specific sequence contexts, while errors are not. For example, tobacco related mutations tend to occur at CC contexts, and mutations related to the activity of APOBEC enzyme prefer the TpC context for inserting somatic mutations (see, Greenman et al., Nature, 446(7132): 153-158, 2007). Thus, sequence context may be used to help identify changes that are more likely to result from sequencing artifacts as well as changes more likely to result from prevalent mutational processes.

With regard to noise cancellation, wherein the marker is a CNV, artefactual noise is cancelled on the basis of a plurality of parameters that are specific to CNVs. In some embodiments, the CNV-specific noise parameter includes “positional attribute” of CNV. Typically, centromere, telomere and/or heterochromatin regions of the chromosome have wide variabilities due to their involvement in rearrangements. CNVs that are located in these regions or proximity thereto (detected via in situ methods as well via computer software), may be disfavored. In some embodiments, the positional attribute of a CNV may be measured based on whether it is at least 1000 kilo bases (kb), at least 400 kb, at least 100 kb, at least 20 kb or fewer kb, e.g., 1 kb from a telomere, centromere, or heterochromatin region of a chromosome. In some embodiments, CNVs located in the subtelomeric region or pericentromeric region, which are characterized by chromosomal rearrangement hotspots, are disfavored. One further feature that may be employed in the methods of the disclosure includes position in read (PTR) or read position. Read position information may be obtained by various techniques using different position measurements, e.g., genomic coordinates of the reads, positions on a reference sequence, or chromosomal positions. In further implementations, unique molecular indices (UMIs) and read positions may be combined to collapse reads.

In some embodiments, the CNV-specific noise parameter includes evaluation of “representativeness” of the CNV with a disease. For instance, previous research has found that CNV calls in immunoglobulin regions are not representative of gDNA and tend to depend substantially on DNA source—e.g., saliva versus blood or lymphoblastoid cell lines versus blood (Need et al., 2009; Wang et al., 2007; Sebat et al., 2004). Such non-representative CNVs may be disfavored.

In some embodiments, the CNV-specific noise parameter includes evaluation of “depth coverage” of the CNV, which refers to the number of unique reads that their mapping overlap a specific genomic coordinate in the CNV genomic segment.

Once noise-markers are filtered, the next step in the diagnostic method comprises integrating genome-wide compendium signal from plasma sample into a mathematical inference model that outputs the estimated fraction of tumor DNA in the biological sample (e.g., plasma). Depending on the marker, the mathematical model integrates a plurality of process quality metrics, as well as patient-specific attributes, to estimate tumor fractions (TF). Recognizing fundamental differences between SNVs (or indels) and CNVs (SVs) with regard to frequency and also associative properties with a trait (e.g., cancer), the systems and methods of the disclosure involve use of marker-specific mathematical algorithms to estimate tumor fractions.

From a workflow perspective, CNV-based detection methods may implement a variation to the SNV-based detection method described previously. In some embodiments, baseline samples (e.g., plasma sample and/or tumor sample) and normal cell sample (e.g., PBMC) are processed separately and also analyzed separately. In the final analytical step, tumor signals are binned separately from PBMC signals, e.g., based on directional coverage skew and local fragment size skew. If the signal is identified as coming from tumor (tumor CNV/SV), then the mathematical model used in estimating tumor fraction has forward directionality; conversely, if the signal is identified as coming from a PBMC, then the mathematical model used in estimating tumor fraction has reverse directionality. Although the tumor fractions may be estimated with tumor samples alone (i.e., without using PBMC samples), the method preferably integrates bi-directionality (i.e., both tumor-based and PBMC-based tumor fraction estimations are integrated).

As in the case of SNV-based detection methods, CNV-based detection methods also allow orthogonal integration of secondary features, e.g., fragment size shifts. Here, the principal method of determining estimated tumor fraction (eTF) using mathematical equations that incorporate directionality features is covered by the provisional application (esp., tumor-based eTF estimation using CNVs). However, to make the prognostic/diagnostic method more robust, accurate, and/or sensitive, read-based features, e.g., shifts in fragment sizes of DNA, may be orthogonally integrated into the model. The significance of the orthogonal features (in determination of MRD) may be determined using a generalized linear model (GLM) to orthogonally determine the tumor fraction based on the relationship between CNV depth coverage and fragment size shift.

In some embodiments, CNV-based methods are carried out as follows: germline markers are removed from baseline samples (typically tumor sample but could also include plasma samples optionally containing tumor samples) and normal samples (typically PBMC). Next, artifact CNV sites are generated over a cohort of healthy plasma samples (panel of normal-PON Blacklist) and are removed from the patient detected mutations in order to remove common sequencing or alignment artifacts such as centromere and repeat regions. Regions-of-interest (ROI) that contains all the genomic segments of the tumor (sT_CNV) and PMBC (sP_CNV) are then binned to discrete windows (500 bp or more) and the depth coverage (read count) in each window is estimated from a follow-up plasma sample (after surgery, during treatment, at follow-up for recurrence). Median depth coverage per window is calculated and divided by the average sample coverage.

Next, depth coverage values are then normalized to correct for GC-content and mappability biases by performing two LOESS regression curve-fitting on the bin-wise GC-fraction and mappability score. Further batch-effect correction is done using a robust-zscore normalization, which is applied to each sample separately. Briefly, median and median-absolute-deviation (MAD) are calculated based on the neutral regions of each sample and then all CNV bins are normalized by (B(i)−Median)/MAD. Next, for each bin the depth coverage skew and fragment size center-of-mass (COM) skew is calculated in comparison to a panel of normal (PON) healthy plasma samples. Herein, low tumor fraction samples show a sparse depth coverage skew that is biased by the directionality of the CNV segment-amplification segment will show a bias towards positive depth coverage skew while deletion show a bias towards negative depth coverage skew. On the other end, neutral regions show a random skew without a preferred directionality, so multiplying the differential (plasma −PON) depth coverage skew by the directionality of the CNV segment (amplification multiplied by +1, deletion multiplied by −1) will sum up the CNV signal across the genome while neutral region noise will be canceled due to random directionality.

This step is performed mathematically and tumor fraction is estimated by checking the linear dilution ratio between the cumulative signals detected at the plasma sample in compare to the cumulative signals detected in the tumor. To address variation in noise between patients with different CNV patterns, patient specific CNV signature are used to calculate the expected noise distribution over a cohort of healthy plasma samples (panel-of-normal, PON). Mainly the same process described above in the case of analysis of SNV markers may be performed to detect the patient specific pattern in healthy plasma samples (PON) or other patients (cross-patient analysis). These detections represent the background noise mode for which the mean and standard-deviation (μ,σ) of artefactual mutation detection rate is calculated. Confidence tumor detection and tumor fraction estimation achieved if the patient detected tumor fraction is higher than a threshold value (e.g., artefactual tumor fraction that correspond to 1.5*σ in error rate above the mean).

It may also be possible to infer tumor fraction from directional genome-wide depth coverage skew in sP_CNV, e.g., using converse methods as described above in the Workflow. Finally, orthogonal features may be integrated into this calculation model to improve the robustness, accuracy, sensitivity or specificity of the algorithms and methods. In some embodiments, the methods of the disclosure include estimation of TF based on detection of a plurality of SNV markers. Herein, estimated TF (eTF[SNV]) is computed by integrating process-quality metrics comprising estimated genomic coverage and sequencing noise with patient specific parameters comprising mutation load (N). Preferably, the method comprises computing an estimated tumor fraction (eTF) for SNV markers, wherein eTF[SNV]=1−[1−(M−E(σ)*R)/N]\hat{ }(1/cov), wherein M is the number of tumor-specific compendium detections in the patient sample, a is a measure of empirically-estimated noise, R is the total number of unique reads in a region of interest (ROI), N is tumor mutation load, and cov is the average number of unique reads per site in the ROI.

In some embodiments, the methods of the disclosure include estimation of TF based on detection of a plurality of CNV markers. Herein, estimated TF (eTF[CNV]) is computed by integrating directional depth of coverage skewed in concordance with tumor CNV directionality wherein amplification of copy number is skewed positively and deletion of copy number is skewed negatively. Preferably, the method comprises computing an estimated tumor fraction (eTF) for CNV markers, wherein eTF[CNV]=(sum_{i}[(P(i)−N(i))*sign[T(i)−N(i)]]−E(sigma))/(sum_{i}[abs(T(i)−N(i))]−E(σ)), wherein P is a median depth value in a genomic window indexed by {i} representing plasma depth coverage, T is a median depth value in a genomic window indexed by {i} representing tumor depth coverage, and N is a median depth value in a genomic window indexed by {i} representing normal depth coverage.

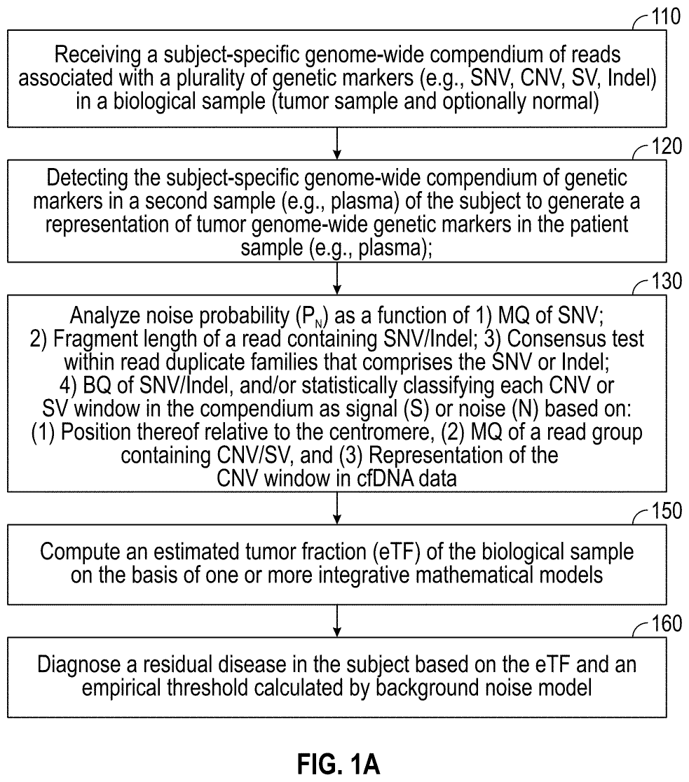

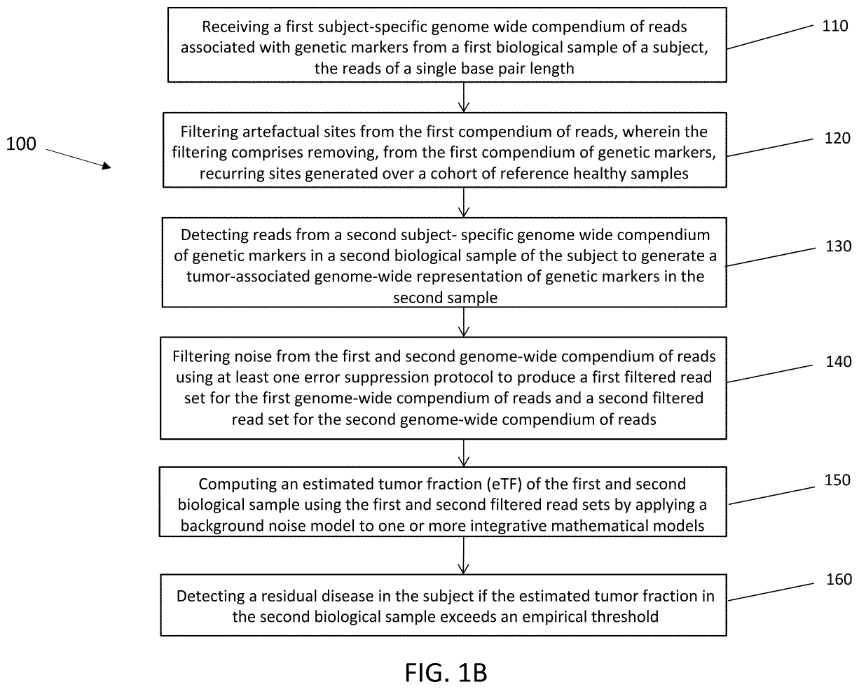

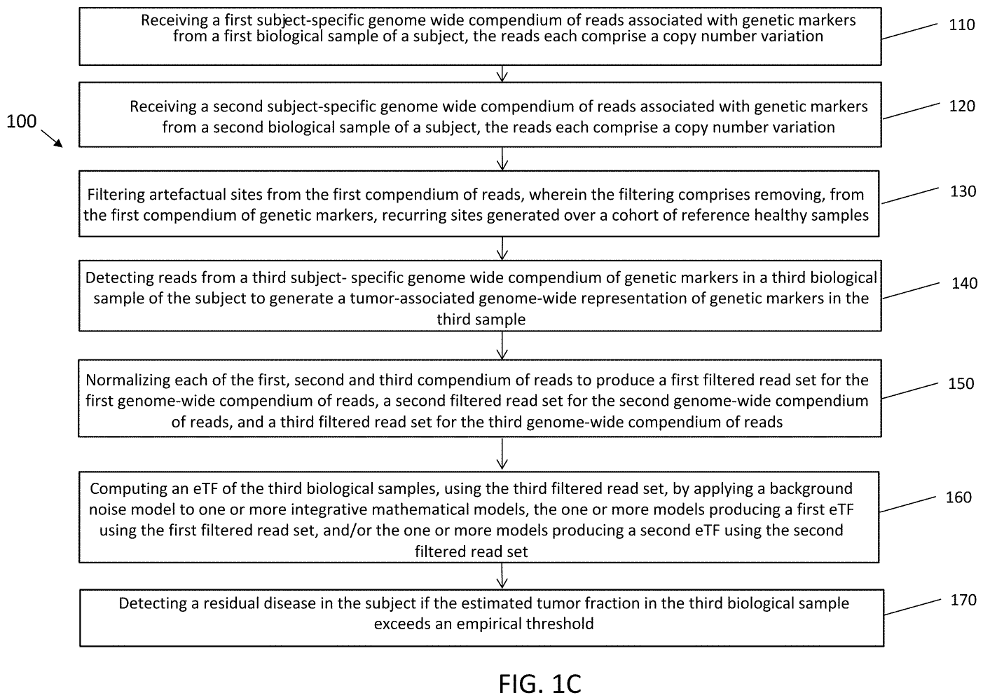

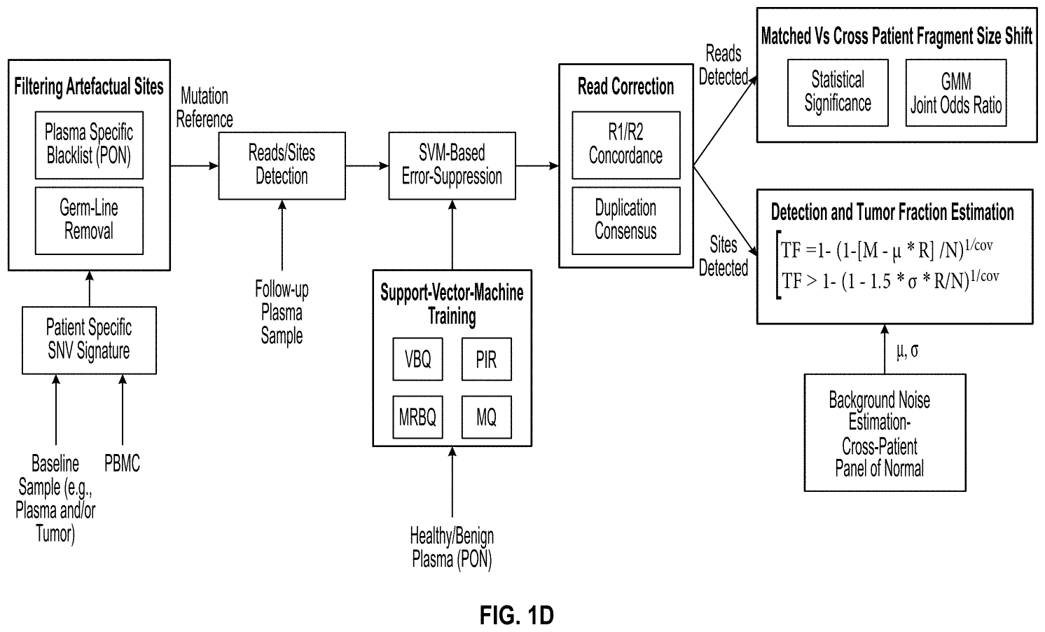

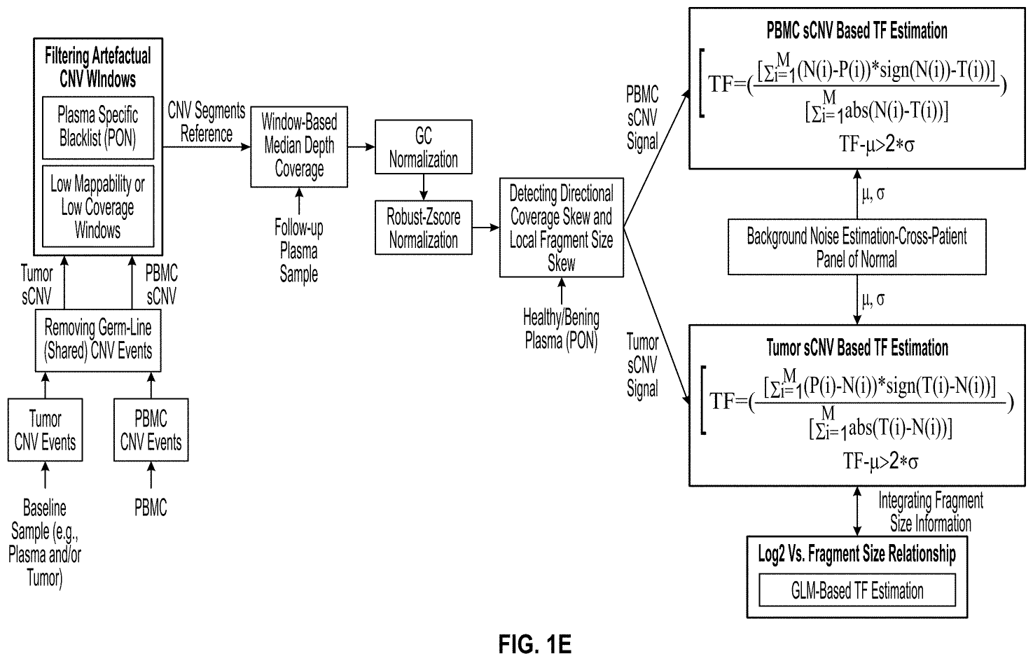

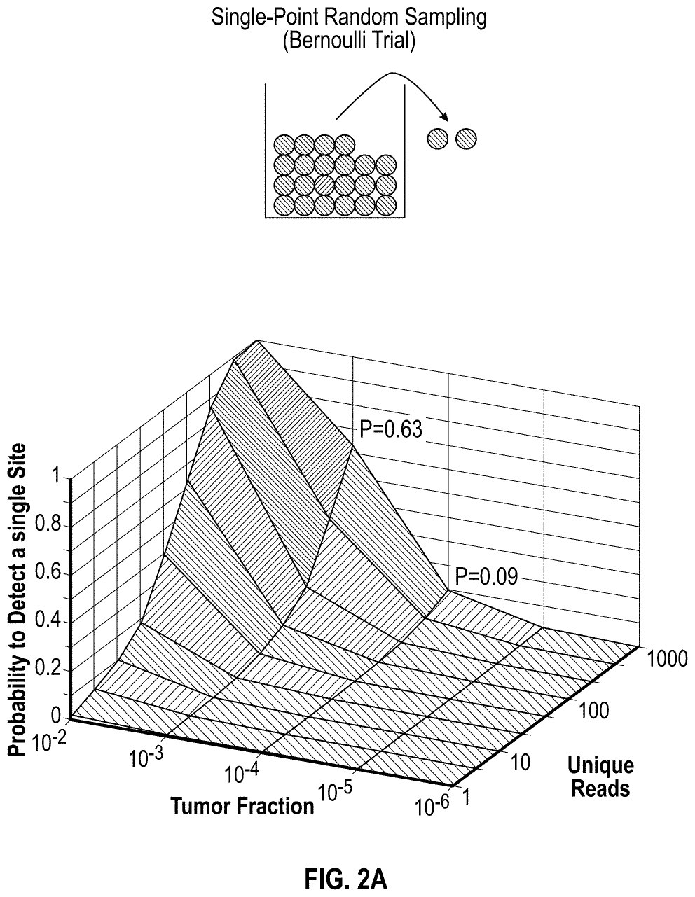

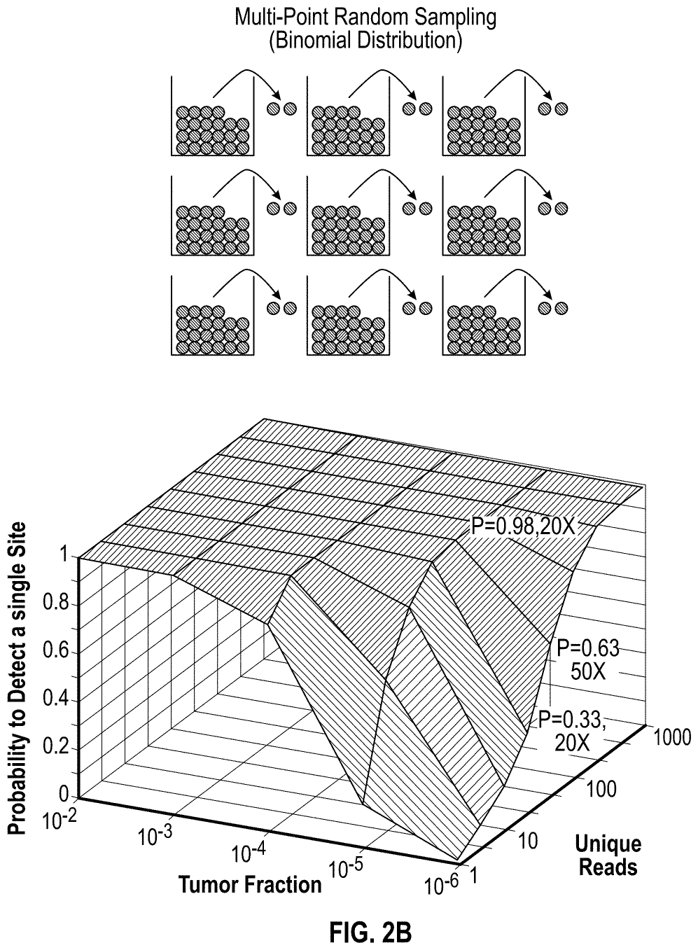

In one aspect, determining TF score may comprise building an optimized base/mapping quality filtration, using optimal receiver operating point to filter SNV noise and analyzing the filtered SNV signals using integrative mathematical models as described above. A representative method is provided in Example 2 and the results are shown in FIG. 2. Error rate distributions may be evaluated across multiple replicates using control samples and also tumor samples. Theoretical threshold values for cutoff may be established using statistical models (e.g., binomial models), against which, empirical measurements are plotted and means/confidence intervals for each measurement are calculated. Noise levels are identified in the distribution using statistical modeling. Baseline tumor fractions (TF), which permit diagnosis of tumors, are established on the basis of statistical measurements. As can be seen in the data of FIGS. 3D to 3G, a tumor fraction above a baseline TF value of about 1×10⁻⁵ is indicative of minimal residual disease for most solid tumors, including, melanoma, lung and breast tumors.

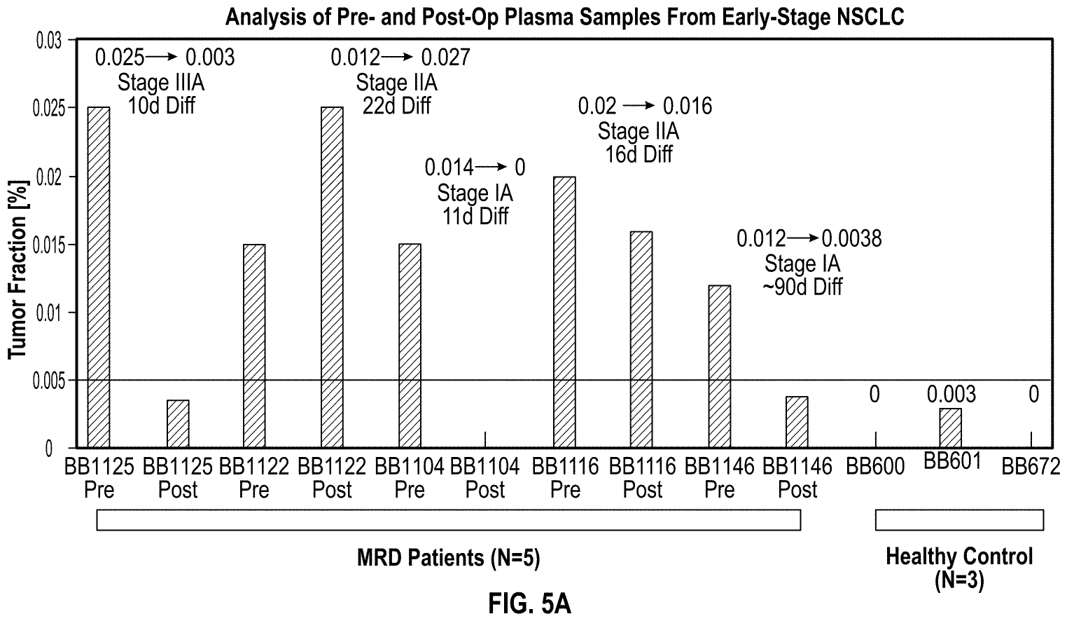

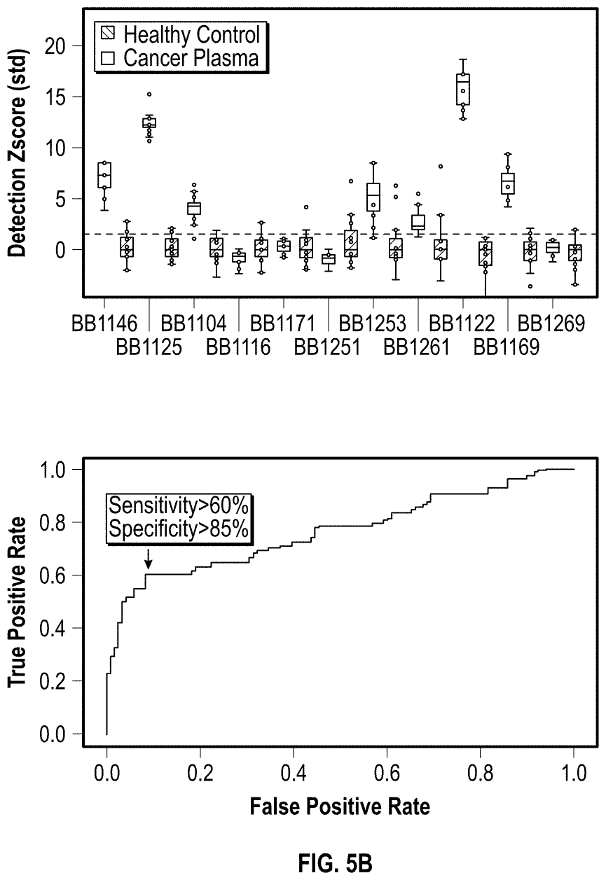

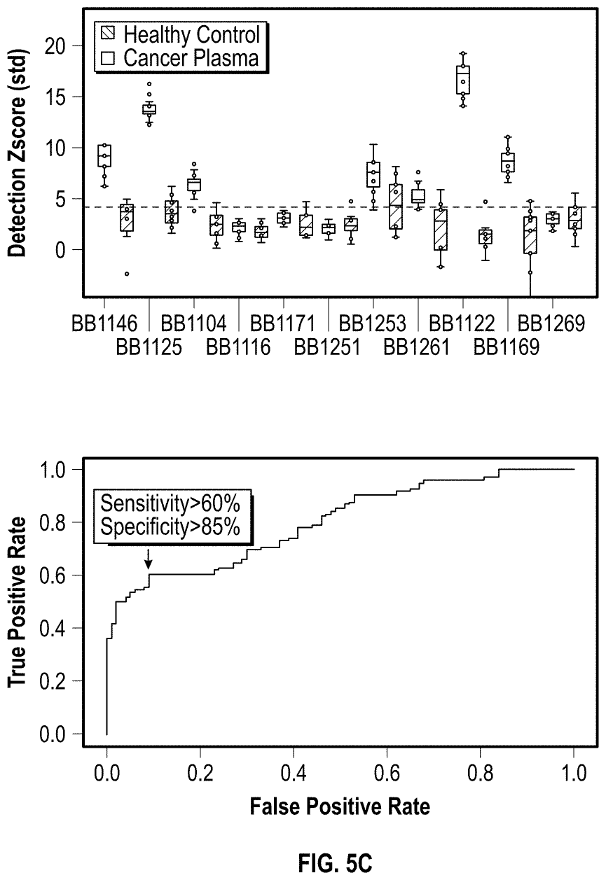

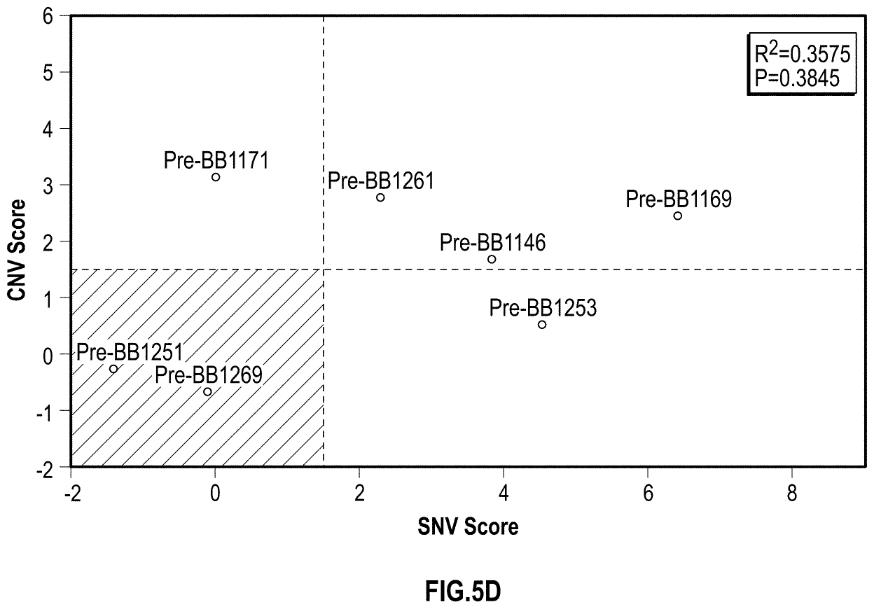

In one aspect, determining TF score may comprise building appropriate filters for filtering CNV noise and analyzing the filtered CNV signals using integrative mathematical models as described above. A representative method is provided in Example 3 and the results are shown in FIG. 5. First, genetic data of resected tumors, germline (e.g., PBMC), and pre-surgery biological sample (preferably, cfDNA) is obtained. A profile of the tumor read-depth, germline read-depth and pre-surgery plasma cfDNA read-depth in a representative amplified segment (e.g., 500 kb; preferably 100 kb) is generated. Depth coverage is normalized across all samples to minimize bias. An integrative mathematical model, which integrates read depth skews across the genome as described above, is employed to evaluate differences between the three sample genomes. The results demonstrate a high detection sensitivity of detection when genome-wide CNV pattern was integrated using the aforementioned methods. More specifically, the methods described above permit a surprising and unexpected ability to detect tumors down to TF of about 1/100,000. This feature is evident from the signal-to noise (SNR) for each TF, where all TFs above 10⁻⁵ show positive (>0) detection of signal compared to noise.

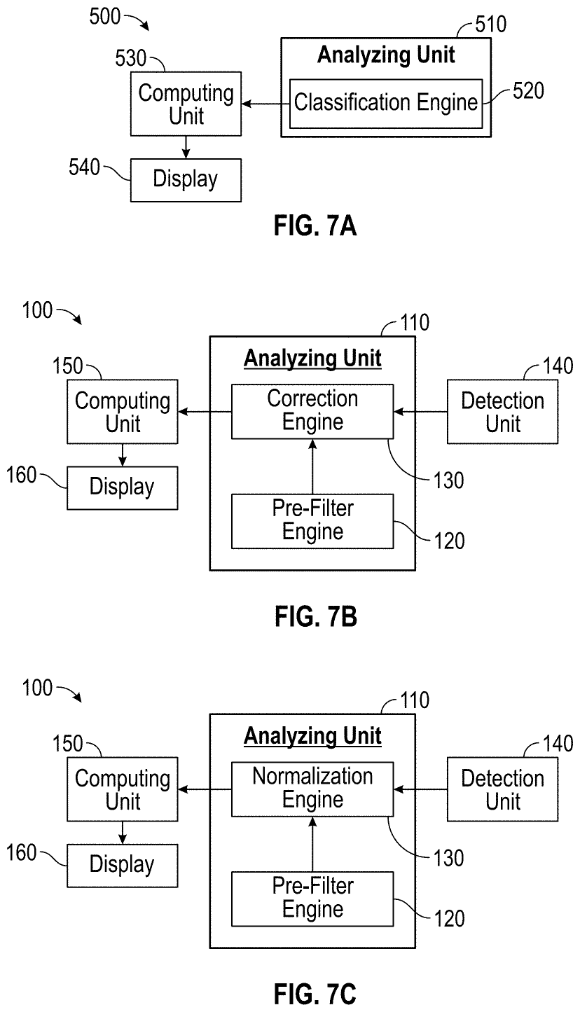

Exemplary systems for use of the methods of the disclosure are shown in FIG. 7A-FIG. 7C. Herein, a compendium of genetic markers is received from a subject (e.g., a cancer patient). The compendium of genetic markers comprises, for example, tumor DNA (e.g., obtained from a resected tumor) and control DNA (e.g., PMBC). The genetic data are analyzed using a mutation caller, and the somatic SNV (sSNV) is set as reference for downstream analysis. In some aspects, this reference standard may be personalized, e.g., to a particular subject. In some aspects, this reference standard may be used together with a cohort of additional reference standards.

Preferably, in order to utilize a very clean and a high-quality reference set, the outputs of three different mutation callers, MUTECT, LOFREQ, and STRELKA, are intersected. MUTECT permits reliable and accurate identification of somatic point mutations in next generation sequencing data of cancer genomes (Cibulskis et al, Nature Biotechnology, 31, 213-219, 2013); LOFREQ models sequencing run-specific error rates to accurately call variants occurring in <0.05% of a population (Wilm et al., Nucleic Acids Res., 40(22): 11189-11201, 2012); STRELKA is an analytical package designed to detect somatic SNVs and small indels from the aligned sequencing reads of matched tumor-normal samples (Saunders et al., Bioinformatics, 28(14):1811-7, 2012).

Typically, mutation caller intersection comprises use of a plurality of art-known callers. In some embodiments, three mutation callers (MUTECT, LOFREQ, and STRELKA) are used on the patient tumor and normal sequencing reads, the intersected variant list is defined as the variant that show the detection of the exact same substitution (same genomic coordinate and nucleotide change) in all callers.

Next, reads from the patient-specific mutation sites are collected and filtered. In some embodiments, the collecting and/or filtering step comprises removing low mapping quality reads. For instance, any read that has a mapping quality score of less than 29 (ROC optimized) is filtered. Additionally or alternately, filtering may involve building duplication families. For instance, duplication may include multiple PCR/sequencing copies of the same DNA fragment (i.e., duplication of markers and region of interest that are not unique). Lastly, a corrected read based on a consensus test may be generated. Filtering step may include removing low base quality reads. For instance, any read that has a base quality score of less than 21 (ROC optimized) may be filtered. Lastly, the filtering step may include removing high fragment size reads For instance, any read that has a fragment size of greater than 160 (ROC optimized) may be filtered. The rationale for this is that tumor DNA tend to be shorter than normal DNA, so low fragment size filter enriches for tumor DNA. See, Jiang et al., PNAS USA, 112.11 (2015): E1317-E1325; and Mouliere et al., bioRxiv, 134437, 2017.

The next step involves computing the number of patient-specific mutation sites that have at least one supporting read (in the filtered set) with the exact same substitution as in the tumor. In some aspects, wherein the marker is SNV, the computation step may include integrating a probabilistic model including: 1) integrated signal of plasma SNV detection, 2) process-quality metrics comprising estimated genomic coverage and sequencing noise model, 3) patient specific parameters comprising mutation load (N). More specifically, the integrated mathematical model may involve computing an estimated eTF[SNV]=1−[1−(M−E(σ)*R)/N]\hat{ }(1/cov), wherein M is the number of tumor-specific SNV compendium detections in the patient plasma sample, a is a measure of empirically-estimated error-rate, R is the total number of unique reads in the SNV compendium region of interest (ROI), N is tumor mutation load, and cov is the average number of unique reads per site in the SNV compendium ROI. Next, the estimated TF is checked against a detection threshold defined by empirically measured basal noise TF estimations from healthy samples. In some aspects, TF is defined as detected if it is above a threshold, e.g., 2 standard deviations of the noise TF distribution (e.g., FPR<2.5%).

In some embodiments, wherein the marker is CNV, the filtration step may include running CNV calling (e.g., analysis of amplification and/or deletions) on tumor and normal (e.g., PBMC) samples from the patient and generating a reference segmentation of all CNV segments that meet the threshold feature (e.g., length of greater than 5 Mega base pairs) along with the directionality of the variation (wherein, amplification is assigned a positive factor, e.g., +1 and deletion is assigned a negative factor, e.g., −1). Next, single base pair depth coverage information for plasma, tumor and PBMC samples covering the patient-specific CNV segmentation ROI is collected. Next, the patient-specific CNV segmentation ROI is normalized to 500 bp windows and the median value per window is calculated for all samples and windows (artifact suppression). Next, normalized depth coverage information for all 500 bp windows is generated.

In some embodiments, normalization may be carried out using (1) robust zscore normalization per sample and/or (2) robust principal component analysis (RPCA) method. For example, the Zscore method may include using the algebraic function preop_median=(preop_median−median(preop_median))./(1.4826*mad(preop_median,1)). Alternately, the robust principal component analysis (RPCA) method may involve solving the optimization problem for M=L+S, for removing noisy and high frequency artifacts (S matrix). A combination of the aforementioned methods may also be used.

Next, reads/windows from the patient-specific segmentation are filtered. In some embodiments, filtration steps may include removing low mapping quality reads (e.g., <29, ROC optimized); removing reads that are in proximity to centromere regions, for example, removing windows with normalized normal value above a threshold (e.g., 10). With respect to the centromere proximity filter, it was identified that ˜70%-80% of CNV noise hotspots co-localized with centromere regions and can be detected by abnormally high depth coverage values in the PBMC samples. These centromere hotspots can be removed in the filtration step.

Next, the non-represented regions in cfDNA are removed. For instance, windows that are not included in a cfDNA representation mask composed from multiple cfDNA samples may be removed. A rationale for this filtration step is that insofar as cfDNA are biased to show only nucleosome protected genomic regions and show non-represented gaps in accessible chromatin genomic regions, inclusion of these non-represented regions into the calculation is likely to cause bias and errors. Accordingly, a mask of the regions that are represented (>0 reads) in the cfDNA cohort are generated using a cohort of cfDNA samples.

Next, computational methods are used to integrate coverage parameters across plasma and normal samples. Accordingly, the directional depth of coverage skewed between plasma and normal (PBMC) patient samples may be integrated using the equation sumᵢ[(P(i)−N(i))*sign[T(i)−N(i)]]−E(sigma). Similarly, the cumulative depth of coverage skewed between tumor and normal (PBMC) patient samples may be integrated using the equation sumᵢ[abs(T(i)−N(i))]−E(σ)).

Next, a dilution ratio between the aforementioned signals, vis-à-vis, directional depth and cumulative depth of coverage, is computed, which corresponds to the estimated tumor fraction (eTF). In some aspects, the computation step may include computing an eTF for CNV markers by utilizing a probabilistic dilution model including: 1) integrating directional depth of coverage skewed between plasma and normal (PBMC) patient samples in concordance with tumor CNV directionality wherein amplification of copy number is skewed positively and deletion of copy number is skewed negatively; 2) integrating the cumulative depth of coverage skewed between tumor and normal (PBMC) patient samples; and 3) finding the dilution ratio between the above signals. More specifically, the integrated mathematical model may involve computing an estimated eTF [CNV]=(sum_{i}[(P(i)−N(i))*sign[T(i)−N(i)]]−E(sigma))/(sum_{i}[abs(T(i)−N(i))]−E(6)), wherein P is a median depth-coverage value in a genomic window indexed by {i} representing plasma depth coverage, normalized by either robust-zscore method or robust PCA compared to a cohort of normal samples; T is a median depth value in a genomic window indexed by {i} representing tumor depth coverage, normalized by either robust-zscore method or robust PCA compared to a cohort of normal samples; and N is a median depth value in a genomic window indexed by {i} representing normal depth coverage, normalized by either robust-zscore method or robust PCA compared to a cohort of normal samples. Next, the estimated TF (CNV) is checked against a detection threshold defined by empirically measured basal noise TF estimations from healthy samples. In some aspects, eTF (CNV) is defined as detected if it is above a threshold, e.g., 2 standard deviations of the noise TF distribution (e.g., FPR<2.5%).

In some embodiments, the probabilistic model is used to compute effective coverage per genomic site based on the mathematical operation A*PBMC_cov+B*tumor_cov, wherein the PBMC coverage and tumor coverage are not the same if a particular site is associated with an amplification or deletion, and A+B=1. In some embodiments, the A, B, for various samples are as follows: control (e.g., PBMC sample) A=1 and B=0; tumor sample B=purity and A=1-purity; plasma sample B=TF and A=1-TF. In some embodiments, the relation between the signal in plasma and tumor is linearly related to the dilution (or change in mixture proportion) between the purity and the TF. As is known in the art, the model is also subjected to noise, which may be included in the probabilistic model.

### Use of the Method in the Therapy of Patients Post-Surgery

The prognosis for cancer patients who have undergone surgical resection of tumors (e.g., breast tumor removal via mastectomy; lung tumor removal via pneumonectomy or lobectomy; or prostatectomy for prostate removal) is of critical importance. For instance, in the breast cancer setting, for women considering adjuvant therapy, a vast majority state a desire to be informed of their prognosis without adjuvant therapy (Ravdin et al., J Clin Oncol., 16(2):515-521, 1998). Adjuvant therapy is undesirable as it is unpleasant and inconvenient (Duric et al., Lancer Oncol., 2(11):691-697, 2001). It may only provide modest benefits in some instances (Simes et al., J Natl Cancer Inst Monogr., 30, 146-152, 2001). Whether or not to have it is a legitimate decision (Duric et al., supra). It may involve tradeoffs Wouters et al. (Ann Oncol., 24(9):2324-9, 2013). There have been calls for refinement of the determination of risk posed by the cancer (Kratz et al., Transl Lung Cancer Res., 2(3): 222-225, 2013).

Many studies note that tumor size is an important prognostic variable. However, in the MRD context, tumor size is not pertinent as the tumors are generally undetectable using traditional diagnostic tools such as CT scans. As such, a cutoff point in tumor size is problematic.

Accordingly, a computerized version of the prediction model would provide an important step in this direction and might be the most accurate prediction method currently available. FIG. 7 illustrates the model predictions in post-surgery patients based on estimated tumor fraction. For instance, an estimated tumor fraction above a threshold value (e.g., about 10⁻⁴ for SNV markers and/or about 10⁻⁵ for SNV markers) would indicate that an adjuvant therapy is needed for the subject.

In addition to its use simply for patient counseling, the model can be useful in a physician's decision regarding adjuvant therapy. Accordingly, the disclosed method provides a tool for physicians and clinicians to predict an outcome (e.g., metastasis or even death) in the absence of adjuvant therapy. Presumably, a patient with a very low baseline risk, as a function of the estimated tumor fraction (eTF) might wish to avoid the toxicity associated with adjuvant therapy. Thus, the prediction tool can be an effective decision aid. This prediction tool might also be useful as a benchmark for judging the predictive ability of any new therapy, such as chemotherapy, immunotherapy or targeted therapy, e.g., using investigational drugs.

### Systems

The disclosure further relates to systems for carrying out the methods of the disclosure. A representative system is provided in the schematic diagram of FIG. 7A, which illustrates an exemplary system for implementing the diagnostic method of the disclosure. As depicted herein, a system 500 is provided that can include analyzing unit 510, a classification unit 520, a computing unit 530, and a display 540 for outputting data and receiving user input via an associated input device (not pictured). Analyzing unit 510 typically comprises an input for genetic data, e.g., a VCF file containing reads from a subject's tumor sample, optionally a normal (e.g., PBMC) sample, and also a second biological sample, e.g., a plasma sample from the same subject (Note: the first and the second sample acquisition may be performed together or sequentially, i.e., temporally separated). Classification unit 520 can include one or more engines for classifying various types of markers, e.g., CNV/SV versus SNP/indels. It should be noted that FIG. 7A illustrates one configuration of a system. The orientation and configuration of these components can vary as needed. Moreover, additional components can be added to this system. These various components, their various operations, their various orientations, and various associations between each other will be discussed in detail below.

In some embodiments, the disclosure relates to a system for detecting residual disease in a subject in need thereof. The system may include an analyzing unit 510 configured and arranged to filter artefactual noise markers from a genome-wide compendium of markers, wherein the genome-wide compendium of markers is generated from a plurality of genetic markers from a biological sample of a subject, the biological sample comprising a tumor sample and a normal cell sample, wherein the compendium of genetic markers is selected from the group consisting of single nucleotide variation (SNV), indels, copy number variation (CNV), structural variant (SV) and combinations thereof, the analyzing unit further comprising detecting the subject-specific genome wide compendium of genetic markers in a second biological sample to generate a representation of tumor genome-wide genetic markers in the second sample, the analyzing unit further comprising a classification engine 520. In some embodiments, the classification engine 520 statistically classifies each marker in the compendium as signal or noise. For instance, wherein the marker is a SNV or indel (grouped together because of similar structural features but it is not necessary to use the same classification scheme), the classification engine classifies the SNV or indel as signal or noise on the basis of probability of detection of noise (P_{N}) as a function of 1) mapping-quality (MQ) of the read group comprises the SNV or Indel, 2) fragment size length of the read group comprises the SNV or Indel, 3) consensus test within read duplicate families that comprises the specific SNV; or 4) base-quality (BQ) of the SNV or Indel. Similarly, wherein the marker is a SNV or indel (grouped together because of similar structural features but it is not necessary to use the same classification scheme), the classification engine classifies the SNV or indel as signal or noise on the basis of 1) position thereof relative to the centromere, 2) mapping-quality (MQ) of the read group comprises the CNV or SV window, or 3) representation of the CNV or SV window in cfDNA data.

In some embodiments, the SNV/indel classification unit 520 statistically classifies each SNV/indel in the compendium as signal or noise on the basis of probability of detection of noise (P_{N}) as a function of base-quality (BQ) of the SNV/indel and mapping-quality (MQ) of the SNV/indel. In some embodiments, the CNV/SV classification unit 520 statistically classifies each CNV/SV in the compendium as signal or noise on the basis of position thereof relative to the centromere, non-representation thereof in a given depth of coverage and read capability thereof. In some embodiments, the classification unit 520 is classifies both SNV/indel markers as well as CNV/SV markers based on one or more of the aforementioned parameters.

In some embodiments, the systems of the disclosure contain a computing unit 530 configured and arranged to calculate estimated tumor fraction (eTF) of the sample on the basis of one or more integrative mathematical models. For example, the computing unit may be configured and arranged to calculate estimated tumor fraction (eTF) of the sample on the basis of one or more integrative mathematical models that is specific to SNV/indel markers or specific to CNV/SV markers. In such embodiments, wherein the marker is SNV/indel, the computing unit may integrate process-quality metrics comprising estimated genomic coverage and sequencing noise with patient specific parameters comprising mutation load (N). Likewise, wherein the marker is CNV or SV, the computing unit may compute an eTF for CNV markers by integrating directional depth of coverage skewed in concordance with tumor CNV directionality wherein amplification of copy number is skewed positively and deletion of copy number is skewed negatively.

The systems of the disclosure further contain a display unit 540 that outputs a residual disease profile of the subject based on the estimated tumor fraction, wherein a residual disease in the subject is output in the residual disease profile if the estimated tumor fraction exceeds an empirical threshold calculated by a background noise model. In some embodiments, in the systems of the disclosure, the classification engine unit and/or the computing unit may be separately or collectively coupled to a display unit that outputs a residual disease profile of the subject based on the estimated tumor fraction.

In some embodiments, the systems 500 of the disclosure comprise an analyzing unit 510 comprising a classification unit 520, which comprises at least one engine selected from the group consisting of an SNV classification engine 520-1, a CNV classification engine 520-2, an indel classification unit 520-3, a structural variant (SV) classification unit 520-4 or a combination thereof 520-5, wherein: the SNV/indel classification engine statistically classifies each SNV in the compendium as signal or noise on the basis of probability of detection of noise (P_{N}) as a function of base-quality (BQ) of the SNV and mapping-quality (MQ) of the SNV; and/or the CNV/SV classification engine statistically classifies each CNV/SV in the compendium as signal or noise on the basis of position thereof relative to the centromere, non-representation thereof in a given depth of coverage and read capability thereof. The system 500 may further comprise a computing unit 530 configured to compute an estimating tumor fraction (eTF) of the sample on the basis of one or more of integrative mathematical models that are specific to the type of marker. For instance, wherein the marker is an SNV, the computing unit 530 may be configured to compute an eTF on the basis of the mathematical model eTF[SNV]=1−[1−(M−E(σ)^{R})/N]\hat{ }(1/cov), wherein M is the number of tumor-specific compendium detections in the patient sample, a is a measure of empirically-estimated noise, R is the total number of unique reads in a region of interest (ROI), N is tumor mutation load, and cov is the average number of unique reads per site in the ROI. Likewise, wherein the marker is an CNV, the computing unit 530 may be configured to compute an eTF on the basis of the mathematical model eTF[CNV]=(sum_{i}[(P(i)−N(i))*sign[T(i)−N(i)]]−E(sigma))/(sum_{i}[abs(T(i)−N(i))]−E(σ)), wherein, P is a median depth value in a genomic window indexed by {i} representing plasma depth coverage, T is a median depth value in a genomic window indexed by {i} representing tumor depth coverage, and N is a median depth value in a genomic window indexed by {i} representing normal depth coverage.

In some embodiments, the computing unit 530 may be configured to compute an eTF on the basis of a mathematical model that is specific to indel (generally similar to or identical to the mathematical model for computing eTF for SNP). In some embodiments, the computing unit 530 may be configured to compute an eTF on the basis of a mathematical model that is specific to SV (generally similar to or identical to the mathematical model for computing eTF for CNV). In some embodiments, the computing unit 530 may be configured to compute an eTF on the basis of a mathematical model that is specific to SNP comprising the equation eTF[SNV]=1−[1−(M−E(σ)^{R})/N]\hat{ }(1/cov), wherein M is the number of tumor-specific compendium detections in the patient sample, a is a measure of empirically-estimated noise, R is the total number of unique reads in a region of interest (ROI), N is tumor mutation load, and cov is the average number of unique reads per site in the ROI and a mathematical model specific to CNV comprising the equation eTF[CNV]=(sum_{i}[(P(i)−N(i))*sign[T(i)−N(i)]]−E(sigma))/(sum_{i}[abs(T(i)−N(i))]−E(σ)), wherein, P is a median depth value in a genomic window indexed by {i} representing plasma depth coverage, T is a median depth value in a genomic window indexed by {i} representing tumor depth coverage, and N is a median depth value in a genomic window indexed by {i} representing normal depth coverage.

In some embodiments, the computing unit 530 is configured to compute an eTF for SNV or Indel markers by integrating a probabilistic model, wherein the probabilistic model comprises 1) integrated signal of plasma SNV or Indel detection, 2) process-quality metrics comprising estimated genomic coverage and sequencing noise model, and/or 3) patient specific parameters comprising mutation load (N); and/or computing an eTF for CNV or SV markers by utilizing a probabilistic mixture model, wherein the probabilistic dilution model comprises 1) integrating directional depth of coverage skewed between plasma and normal patient samples in concordance with tumor CNV or SV directionality wherein amplification of copy number is skewed positively and deletion of copy number is skewed negatively; 2) integrating the cumulative depth of coverage skewed between tumor and normal patient samples; and/or 3) finding a dilution ratio between the above signals.

In accordance with various embodiments herein, a computer readable medium is provided, the computer readable medium comprising computer-executable instructions, which, when executed by a processor, cause the processor to carry out a method or a set of steps for filtering noise in a compendium of genetic markers received from a subject's sample, wherein the genetic markers comprise SNVs (preferably sSNVs), CNVs (preferably sCNVs), indels, and/or SV (preferably translocations, gene fusions or combinations thereof) in a genomic read. Preferably, the filter removes artefactual noise markers from the genome-wide compendium of markers by statistically classifying each SNV or Indel in the compendium as signal or noise on the basis of probability of detection of noise (P_{N}) as a function of 1) mapping-quality (MQ) of a read group comprising the SNV, 2) fragment size length of a read group comprising the SNV, 3) consensus test within read duplicate families that comprises the SNV or Indel, 4) base-quality (BQ) of the SNV or Indel; and/or by statistically classifying each CNV or SV window in the compendium as signal or noise on the basis of 1) position thereof relative to the centromere, 2) mapping-quality (MQ) of the read group comprising a CNV or SV window, 3) representation of the CNV window in cfDNA data. The computer readable medium may further comprise computer-executable instructions, which, when executed by a processor, cause the processor to carry out a method or a set of steps for computing an estimated tumor fraction (eTF) of the biological sample on the basis of one or more integrative mathematical models; and diagnosing a residual disease in the subject based on the estimated tumor fraction and an empirical threshold calculated by background noise model.

In some embodiments, the system comprises a computing unit 530 comprising computer-executable instructions, which, when executed by a processor, cause the processor to carry out a method or a set of steps for estimating tumor fraction (eTF) based on one or more of the aforementioned mathematical models for computing eTF; and a diagnosing unit that makes a qualified diagnosis based on the computed eTF (e.g., if eTF≥2 std above a noise-threshold, then a positive diagnosis is made). The system may further comprise a display 540 for outputting data and receiving user input via an associated input device (e.g., mouse). In some embodiments, the results may be displayed on display 540 in the form of a binary output (i.e., “+ve for MRD” or “−ve for MRD”) or an ordinal score, e.g., in a scale of 1 to 5; wherein a score of 1 indicates that it is unlikely that the subject has MRD and a score of 5 indicates that it is likely that the subject has MRD.

As illustrated in FIG. 7B, an example system 100 is provided that is configured and arranged to detect residual disease in a subject in need thereof. Referring to FIG. 7B, system 100 can comprise an analyzing unit 110 and a computing unit 150. Analyzing unit 110 can comprise a pre-filter engine 120 and a correction engine 130. These system components and associate engines will be discussed in more detail below.

Referring again to FIG. 7B, pre-filter engine 120, of analyzing unit 110, can be configured and arranged to receive a first subject-specific genome wide compendium of reads associated with genetic markers from a first biological sample of a subject. As discussed with regards to workflows herein, and in accordance with various embodiments, the first biological sample can comprise a baseline sample; the first compendium of reads can each comprise reads of a single base pair length; the baseline sample can comprises a tumor sample or a plasma sample.

Pre-filter engine 120 in FIG. 7B can also be configured and arranged to filter artefactual sites from the first compendium of reads. As discussed with regards to workflows herein, and in accordance with various embodiments, the filtering can comprise removing, from the first compendium of genetic markers, recurring sites generated over a cohort of reference healthy samples, and/or identifying germ line mutations in peripheral blood mononuclear cells of the normal cell sample and removing said germ line mutations from the from the first compendium of genetic markers.

In FIG. 7B, correction engine 130, of analyzing unit 110, can be configured and arranged to receive output from engine 120. Correction engine 130 can also be configured and arranged to receive reads from a second subject-specific genome wide compendium of genetic markers in a second biological sample of the subject to generate a tumor-associated genome-wide representation of genetic markers in the second sample. As illustrated in to FIG. 7B, the reads for the second biological sample can be detected using a detection unit 140. Said detection unit 140 can be part of system 100 or not part of system 100, in which case, the reads can be simply received by correction engine 130 from outside system 100. Moreover, these reads can be received into analyzing unit 110 at any point in the system prior to noise filtering as will be discussed below. Moreover, these reads can even be received after noise filtering if the reads are provided to system 110 with noise already filtered out. Moreover, detection unit 140 can be integrated into analyzing unit 110 or be separate from analyzing unit 110, as illustrated in to FIG. 7B.

Correction engine 130 can also be configured and arranged to filter noise from the first and second genome-wide compendium of reads using at least one error suppression protocol to produce a first filtered read set for the first genome-wide compendium of reads and a second filtered read set for the second genome-wide compendium of reads.

As discussed with regards to workflows herein, and in accordance with various embodiments, the at least one error suppression protocol can comprise calculating the probability that any single nucleotide variation in the first and second compendium is an artefactual mutation, and removing said mutation.

As discussed with regards to workflows herein, and in accordance with various embodiments, the probability can be calculated as a function of features selected from the group consisting of mapping-quality (MQ), variant base-quality (MBQ), position-in-read (PIR), mean read base quality (MRBQ), and combinations thereof.

As discussed with regards to workflows herein, and in accordance with various embodiments, the at least one error suppression protocol can include removing artefactual mutations using discordance testing between independent replicates of the same DNA fragment generated from polymerase chain reaction or sequencing processing, and/or duplication consensus wherein artefactual mutations are identified and removed when lacking concordance across a majority of a given duplication family.

Computing unit 150, of system 100, can be configured and arranged to receive output from correction engine 130, and compute an estimated tumor fraction (eTF) of the first and second biological sample using the first and second filtered read sets by applying a background noise model to one or more integrative mathematical models. Computing unit 150 can be further configured and arranged to detect a residual disease in the subject if the estimated tumor fraction in the second biological sample exceeds an empirical threshold. The background noise model, integrative mathematical models, and empirical threshold are discussed in detail herein.

System 100 can also include display 160, as illustrated in to FIG. 7B. The display can be configured and arranged to receive output from computing unit 150. Output can include data related to detection of residual disease in the subject/user. Alternatively, system 100 may exclude a display and can instead send data output from computing unit 150 to any form of storage or display device or location external to system 100. As also discussed herein, the components of system 100 can be integrated into one single unit or can be split up into more separate physical units than that which is illustrated in to FIG. 7B. Moreover, system 100 can be part of a distributed network of systems each performing substantially similar tasks and transmit data from each system to a hub.

As illustrated in FIG. 7C, an example system 100 is provided that is configured and arranged to detect residual disease in a subject in need thereof. Much like the example system of FIG. 7C, system 100 can comprise an analyzing unit 110 and a computing unit 150. As opposed to the system of FIG. 7B, analyzing unit 110 of FIG. 7C can comprise a pre-filter engine 120 and a normalization engine 130. These system components and associate engines will be discussed in more detail below.

Referring again to FIG. 7C, pre-filter engine 120, of analyzing unit 110, can be configured and arranged to receive a first subject-specific genome wide compendium of reads associated with genetic markers from a first biological sample of a subject. As discussed with regards to workflows herein, and in accordance with various embodiments, the first biological sample can comprise a baseline sample; the first compendium of reads can each comprise reads of a single base pair length; the baseline sample can comprises a tumor sample or a plasma sample.

Pre-filter engine 120 can also be configured and arranged to receive a second subject-specific genome wide compendium of reads associated with genetic markers from a second biological sample of a subject. As discussed with regards to workflows herein, and in accordance with various embodiments, the second biological sample can comprise a peripheral blood mononuclear cell sample (PBMC); the second compendium of genetic markers can each comprise a copy number variation (CNV).

Pre-filter engine 120 can also be configured and arranged to filter artefactual sites from the first and second compendium of reads. As discussed with regards to workflows herein, and in accordance with various embodiments, the filtering can comprise removing, from the first and second compendium of reads, recurring sites generated over a cohort of reference healthy samples; identifying shared CNVs between the first and second compendium as germ line mutations and removing said mutations from the first and second compendium of reads.

Normalization engine 130, of analyzing unit 110, can be configured and arranged to receive output from engine 120. Normalization engine 130 can also be configured and arranged to receive reads from a third subject-specific genome wide compendium of genetic markers in a third biological sample of the subject to generate a tumor-associated genome-wide representation of genetic markers in the second sample.

As illustrated in FIG. 7C, the reads for the third biological sample can be detected using a detection unit 140. Said detection unit 140 can be part of system 100 or not part of system 100, in which case, the reads can be simply received by normalization engine 130 from outside system 100. Moreover, these reads can be received into analyzing unit 110 at any point in the system prior to noise filtering as will be discussed below. Moreover, these reads can even be received after noise filtering if the reads are provided to system 110 with noise already filtered out. Moreover, detection unit 140 can be integrated into analyzing unit 110 or be separate from analyzing unit 110, as illustrated in FIG. 7C.

Normalization engine 130 can also be configured and arranged to normalize each of the first, second and third compendium of reads to produce a first filtered read set for the first genome-wide compendium of reads, a second filtered read set for the second genome-wide compendium of reads, and a third filtered read set for the third genome-wide compendium of reads. Normalization methods are discussed in detail herein and can be used in any contemplated combination to normalize reads as discussed.

Computing unit 150 of system 100 in FIG. 7C can be configured and arranged to receive output from normalization engine X30, and compute an estimated tumor fraction (eTF) of the third biological samples, using the third filtered read set, by, for example, applying a background noise model to one or more integrative mathematical models, the one or more models producing a first eTF using the first filtered read set, and/or the one or more models producing a second eTF using the second filtered read set. Computing unit 150 can be further configured and arranged to detect a residual disease in the subject if the estimated tumor fraction in the third biological sample exceeds an empirical threshold. The background noise model, integrative mathematical models, and empirical threshold are discussed in detail herein.

System 100 can also include display 160, as illustrated in FIG. 7C. The display can be configured and arranged to receive output from computing unit 150. Output can include data related to detection of residual disease in the subject/user. Alternatively, system 100 may exclude a display and can instead send data output from computing unit 150 to any form of storage or display device or location external to system 100. As also discussed herein, the components of system 100 can be integrated into one single unit or can be split up into more separate physical units than that which is illustrated in FIG. 7C. Moreover, system 100 can be part of a distributed network of systems each performing substantially similar tasks and transmit data from each system to a hub.

### Other Related Embodiments

Estimation of Transplant Rejection

The disclosure further relates to estimation of transplant rejection estimation using the aforementioned systems, methods and algorithms. Preferably, transplant rejection may be estimated using the SNV/indel-based workflows outlined in FIG. 1B and FIG. 1D.

In some embodiments, estimation of transplant rejection is based on a protocol that utilizes a reference of SNPs that are specific only to the donor (and which do not appear in the recipient). Based on the detection rate of these donor-specific SNPs in the recipient's blood (e.g., post-transplantation), donor-DNA fractions may be calculated using the methods and systems of the disclosure. The donor-DNA fractions are expected to be correlated with the apoptosis rate or rejection rate of the transplanted tissue. For e.g., high donor-DNA fraction is associated with high rejection phenotype; low donor-DNA fraction is associated with low rejection phenotype.

In some embodiments, a differential SNP between donor and recipient, as measured using the methods of the disclosure, may be used to estimate fraction of donor DNA (eDF) in the recipient's blood sample. The odds/likelihood that the transplant is going to be rejected is calculated based on the eDF. For e.g., if the eDF is greater than a certain threshold, then it indicates that the transplanted tissue is going to be rejected by or be incompatible with the host. Conversely, if the eDF is at or below the threshold level, then it indicates that the transplanted tissue is going to be accepted by or be compatible with the host.

Noninvasive Prenatal Testing (NIPT) of Chromosomal Aberration

The disclosure further relates to noninvasive prenatal testing (NIPT) of chromosomal aberration using the aforementioned systems, methods and algorithms. Preferably, NIPT may be carried out using the CNV/SV-based workflows outlined in FIG. 1C and FIG. 1E. Herein, known amplifications and deletions are used as the CNV reference set against which a subject's sample (e.g., amniotic fluid or blood obtained from a pregnant female carrying a fetus suspected of having chromosomal aberration) is measured against. The workflows in FIG. 1C and FIG. 1E are designed to detect changes in copy-number-variation even if the signal is low and sparse, assuming that the segment and directionality (amplification, deletion) of interest are known. In the NIPT context, assuming a test for trisomy 21 in the mother blood is of interest, the segment of interest (chromosome 21) and the direction of change (amplification) are both known.

### EXAMPLES

The structures, materials, compositions, and methods described herein are intended to be representative examples of the disclosure, and it will be understood that the scope of the disclosure is not limited by the scope of the examples. Those skilled in the art will recognize that the disclosure may be practiced with variations on the disclosed structures, materials, compositions and methods, and such variations are regarded as within the ambit of the disclosure.

### Example 1: Methods and Systems for Detection and Validation of Tumor-Specific Low-Abundance Tumor Markers and Use of the Same in Cancer Diagnostics

The systems and methods of the disclosure are useful in the detection of minimal residual disease. As is known in the art, in contrast to metastatic cancer (which is characterized by a high disease burden and significantly elevated ctDNA), in the setting of residual disease detection, ctDNA abundance limits the use of targeted sequencing technology. Given the known limited amount of cfDNA in the setting of low tumor burden, firstly, the potential of optimization of cfDNA extraction was investigated. First, to reduce variation derived from sample acquisition and inter-individual variation, commercially-available extraction kits and methods were compared using uniform cfDNA material generated through large-volume plasma collections (about 300 cc) through plasmapheresis of healthy subjects and cancer patients undergoing hematopoietic stem cell collection. The large volume of plasma allows the testing of multiple methods and protocol parameters on the same cfDNA input, enabling accurate measurement of subtle differences in yield and quality.

Kits and/or extraction methods from Capital Biosciences (Gaithersburg, MD, USA; Catalog #CFDNA-0050), Qiagen (Germantown, MD, USA), Zymo (Irvine, CA, USA; Catalog #D4076), Omega BIO-TEK (Norcross, GA, USA; Catalog #M3298), and NEOGENESTAR (Somerset, NJ, USA, Catalog #NGS-cfDNA-WPR) were used in this comparative study. These kits and reagents were uniformly utilized as per the manufacturer's instructions to perform extraction on 1 ml of the large-volume plasma sample. Multiple plasma aliquots were processed in parallel to assess both inter- and intra-method variability. The yield and purity of each recovered cfDNA sample was determined using fluorescence quantification (total mass), UV absorbance (detection of salt and protein contaminants), and on-chip electrophoresis (size distribution and gDNA contamination).

The results demonstrate that the MAG-BIND cfDNA Extraction Kit from Omega BIO-TEK outperformed all the other tested methods. A systematic optimization of each step of the manufacturer's protocol was further performed so as to reduce contaminant carryover and to improve the recovery of the cfDNA. Even then, cfDNA yield in early stage NSCLC (n=21) remained low and highly variable (median 5 ng/ml (<1000 genomic equivalents); range 3-30 ng/ml).

The above data supports the notion that detection of a single point mutation in a patient's plasma sample results from two consecutive statistical sampling processes: (i) the probability that the mutated fragment is sampled in the limited number of genomic equivalents present in a typical plasma sample, and (ii) the probability of detecting the mutated fragment in the sample given its abundance, sequencing depth and sequencing error (signal-to-noise). While the latter process has been at the focus of intensive investigation and technology development by the scientific community (e.g., ultra-deep error free sequencing protocols), the former stochastic process is infrequently addressed. Nevertheless, in low disease burden ctDNA detection, both processes play a critical role as shown in FIG. 2. If no physical fragments are present that contain the targeted point mutation, even ideal ultra-deep targeted sequencing will fail to discover the cancer signal. In practice this problem is further compounded by the fact that a single observation (mutated sequencing read) is rarely sufficient for confident detection.

Thus, the genomic equivalents present in a plasma sample constitute a random sampling of the entire pool of cfDNA fragments in the patient's circulation, which can be formulated by the Bernoulli trial random sampling model. This model predicts that the detection probability in TFs relevant to the early stage cancer regime (TF<1%), will exhibit a rapid decrease for low TF. Even at a frequency of 0.1% ( 1/1000), detection probability is predicted to be lower than 0.65 (FIG. 2A). However, introducing breadth of sequencing can compensate for the limited coverage per site (a function of limited genomic equivalents), by virtue of repeating the Bernoulli trial on large number of sites. Utilizing this model, it was found that integrating over 20,000 point mutations (˜10 mutations/mb found in 17% of human cancer) 11 can provide a high detection probability (up to 0.98) even at TF of 1:100,000, at a modest sequencing effort (e.g., 20× coverage, FIG. 2B), such that can be readily achieved with standard whole genome sequencing (WGS).

The optimized extraction protocol was then applied to patient samples. This cohort includes 6 post-surgery (˜14 d) plasma samples from the same patients for minimal residual disease (MRD) estimation, and 4 plasma samples from benign patients (control). Despite optimized extraction, cfDNA yield in the low disease burden samples remained low and showed high variability between patients ranging between 0.13 ng/mL to 1.6 ng/mL. These data confirm the low and variable number of DNA molecules available for cfDNA sequencing.

Collectively, these results demonstrate that in the setting of MRD detection, limited input material constitutes a major barrier to the effective application of ultra-deep targeted sequencing (minimal ctDNA frequency of 0.1-1%) given that number of genomic equivalents is well below the depth of sequencing applied.

### Example 2: Genome Wide Integration Allows Sensitive WGS Based NSCLC ctDNA Detection of Residual Post-Surgical Disease for Adjuvant Therapy Stratification and Therapeutic Optimization

Ultra-sensitive identification of MRD with cfDNA may have fundamental prognostic implications and allow the stratification of patients for follow-up adjuvant chemotherapy. Current approaches largely seek to extend the paradigm of mutation detection of driver hotspots through increasing the depth sequencing to counter the low fraction of ctDNA in cfDNA. Nevertheless, these approaches are inherently limited by the ceiling of genomic equivalents. To overcome this limitation, genome-wide information was integrated, reasoning that pooling information across the genome will allow capitalizing on the high mutation rate in lung cancer. Accordingly, instead of relying on deeper sequencing of few sites, the breadth of mutation detection was extended across the genome to increase sensitivity. Thus, WGS was applied to base sensitive detection on the cumulative signal provided by 10,000-30,000 somatic mutations observed in a substantial proportion of NSCLC. Notably, the majority of these mutations is thought to occur prior to transformation and therefore they are likely present even in early stage NSCLC. To evaluate this approach for residual disease detection in NSCLC patients after surgery for curative intent, five early-stage lung cancer patient samples were analyzed (full clinical details are provided in Table 1).

TABLE 1
Clinical information for the currently sequenced patients.

| | | | | | Cigarette | | TUMOR | | |
|---|---|---|---|---|---|---|---|---|---|
| | | | | | Pack | CANCER | SIZE | | Days between |
| Histology | AGE | SEX | ETHNICITY | SMOKER | Years | STAGE | (CM) | Comments | blood samples |
| Adenocarcinoma | 72 | F | Unknown | Former | 14 | IA | 2.6 | | 11 |
| Adenocarcinoma | 69 | M | White | Former | 38 | IIA | 2.1 | | 22 |
| Adenocarcinoma | 87 | F | Unknown | Former | 15 | IIIA | 4.4 | | 10 |
| Adenocarcinoma | 79 | M | White | Former | 30 | IIA | 2.3 | | 16 |
| Adenocarcinoma | 73 | F | | Never | 0 | IA | 3 | | 90 |
| Benign process | 75 | M | Unknown | Current | 50 | NA | NA | COPD | |
| Benign process | 70 | F | Unknown | Former | 20 | NA | NA | Bronchiectasis | |
| Benign process | 64 | M | Unknown | Former | 30 | NA | NA | ILD. | |

First WGS was performed on matched tumor DNA and germline DNA from peripheral blood mononuclear cells (PBMC) to generate patient-specific genome-wide sSNV compendiums. In addition, plasma samples were collected before surgery and at about 14 days after surgical resection. cfDNA was extracted according to the optimized MAG-BIND cfDNA Extraction Kit and library was prepared from only 1 ng of patient cfDNA according to the kit.

Next, the MRD was detected using point mutation pattern matching. To this, robust mathematical models were built to estimate tumor fractions for SNV markers as well as CNV markers. The mathematical models indicate that increasing the number of sites will result in a significant increase of detection probability. To validate this prediction, detection of cfDNA was simulated using in silico mixtures of tumor and normal WGS data from multiple lung adenocarcinoma patients, by admixing tumor and normal WGS reads in varying proportions to obtain virtual plasma samples of different TFs (10⁻² to 10⁻⁶, n=5 replicates each). To simulate noise and possibly false detections a complementary dataset of sequencing reads was generated from matched normal germline WGS without admixture of tumor reads (TF=0, n=20 replicates). To simulate detection in the residual disease setting, somatic mutation calling was performed on the original tumor and germline WGS data, and obtained a patient-specific compendium of somatic SNVs. Then the number of tumor-associated mutated sites in the in silico plasma simulation mixtures was measured through detection of at least one supporting read for the patient-specific SNV compendium. By analyzing simulated plasma with and without ctDNA, it was identified that sequencing noise is the major barrier for sensitive detection. To reduce the impact of sequencing artifacts, errors associated with lower Base-Quality (BQ) and Mapping-Quality (MQ) markers were filtered. A joint BQ and MQ optimized filter was developed through optimal receiver point analysis (ROC, FIG. 3A), which reduced the measured error rate by −10 fold (to about 2/10,000, FIG. 3B). Collectively, this optimized SNV detection method show high agreement between our proposed mathematical method (red line, FIG. 3C) and measured empirical data (mean+/−confidence interval, FIG. 3C), as well as high sensitivity approaching TF= 1/100,000. Moreover, the high agreement between the experimental results and the mathematical model enabled us to accurately transform empirical SNV detection to TF estimates (FIG. 3D), allowing for quantitative MRD monitoring. Moreover, in silico validation of TF estimation shows that accurate and specific estimations were obtained to all TF above 5×10⁻⁵ (FIG. 3E, FIG. 3F and FIG. 3G). Here, a high correlation (R²=0.999) was observed between input mixture TF (x-axis) and TF estimated from the mutation pattern (y-axis) in three different samples, e.g., melanoma (FIG. 3E), lung (FIG. 3F) and breast (FIG. 3G) tumor samples.

The data show that the filters reduce noise in the sample. For instance, pre-filter noise occurs at a rate of ˜2×10⁻³ for both lung and melanoma cancer types, post filter noise rate decrease to ˜2×10⁻⁴ for both cancer types (FIG. 3C). Application of a joint Base Quality (BQ) and Mapping-Quality (MQ) optimized filter with alleviated 35× coverage permitted detection of markers in samples having a TF as low as 1/20,000. Here, red line represents theoretical (binomial model) expectation and empirical measurements are shown in black (mean & confidence interval for 5 independent replicates (FIG. 3D). Noise level is represented by the gray area according to TF=0 detection distribution. Further, in in silico validation of TF estimation in melanoma samples, accurate and specific estimations were obtained to all TF above 5×10⁻⁵ (FIG. 3E).

Analytical validation of markers using synthetic plasma mixtures further demonstrates the validity of both somatic SNVs and somatic cCNVs in tumor fraction estimation at all TF>5×10⁻⁵, and especially at TF>5×10⁻⁴. Data are shown in FIG. 3H and FIG. 3I.

Further analytical validation of the methods using synthetic samples shows a very good correlation between SNV and CNV method of detection (R²=83.5%). See, FIG. 3J.

Comparative assessment of the methods of the present disclosure compared to ICHOR shows that the ICHOR method provides correlation between inputted tumor fraction and output tumor fraction only when TF>5×10⁻³ (FIG. 3K).

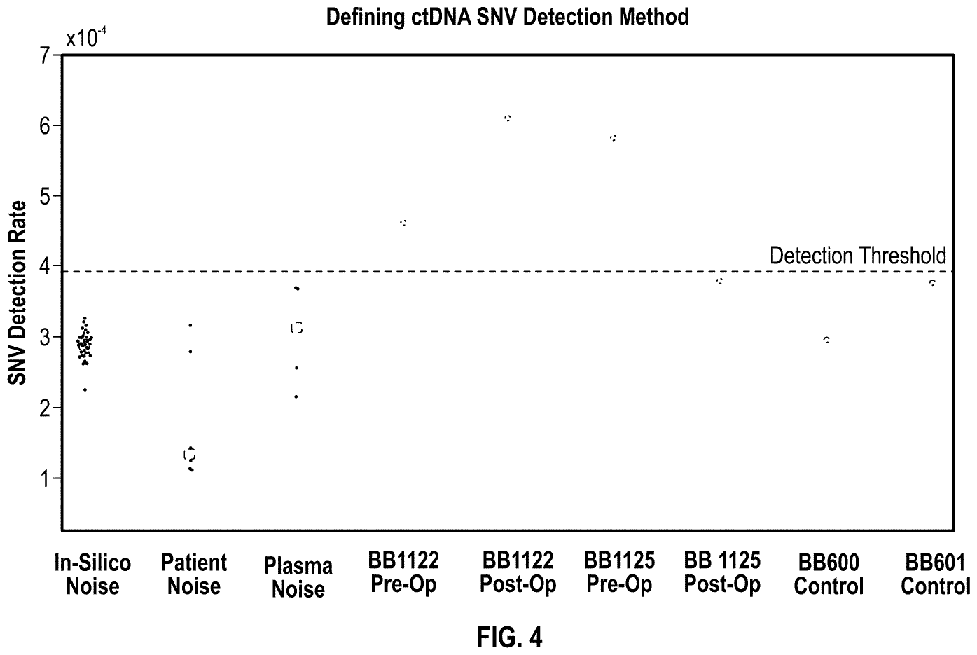

A graph showing SNV detection rates in ctDNA samples obtained in silico or from control subjects (BB601) or cancer patients (BB1122 or BB1125) using the methods and systems of the disclosure is presented in FIG. 4.

To evaluate the approach for residual disease detection in NSCLC patients after surgery for curative intent, five early-stage lung cancer samples were collected (Table 1). First WGS was performed on matched tumor and germ line DNA (PBMC) to generate patient-specific genome-wide SNV compendiums. In addition, plasma sample was collected from subjects before surgery and at about 14 days after surgical resection. CfDNA was extracted and sequenced through the optimized WGS protocol, followed by analysis of SNV detection in all plasma samples based on their patient-specific genome-wide SNV compendiums.

The results are presented in FIG. 5A. The data shows genome-wide SNV detection above the noise threshold in all 5 pre-operative plasma samples of early stage NSCLC adenocarcinoma cases (FIG. 5A). Moreover, post-operative plasma detection was noted in 2 out of 5 patients, in correlation with clinical outcome (recurrence or death) for these patients (FIG. 5A). Specifically, only two patients show post-surgery TF above the noise threshold of 5×10⁻⁵. However, all healthy control samples show TF below the detection threshold. N.D. denotes not detected. The data shows concordant results with the SNV method in terms of plasma detection and TF correlation.

To clinically validate this innovative approach and facilitate its implementation in clinical practice, the aforementioned methodology is applied in 30 cases of early stage lung cancer (stage I and II). First WGS is performed on matched previously collected tumor and PBMC DNA for these patients, as well as pre and post-operative plasma samples. SNV based detection algorithm is used to quantify the pre- and post-operative TF. Clinical variables that are associated with high pre- or post-operative plasma TF (e.g., stage of disease, lymph node involvement, pathological features, and demographic information of the patient) are identified. The impact of positive post-operative plasma sample on the progression-free survival of these patients is specifically examined. Data from a representative cohort of 11 patients are shown in FIG. 5B (adenocarcinoma against healthy plasma control) and FIG. 5C (adenocarcinoma against cross-patient negative control), indicating sensitivity of >60% and specificity of >85%. Concordance between sSNV and sCNV detection is shown in FIG. 5D.

Post-surgery tumor DNA detection can be used as a prognostic marker for aggressive disease that require adjuvant therapy. For instance, in a post-surgery (plasma collected 2 weeks after surgery) analysis of the outcome of 11 patients, relapse-free time was found to be inversely associated with sSNV-based zscore detection (FIG. 1111).

### Example 3A: Orthogonal Integration of Fragment Size Features in SNV-Based Methods

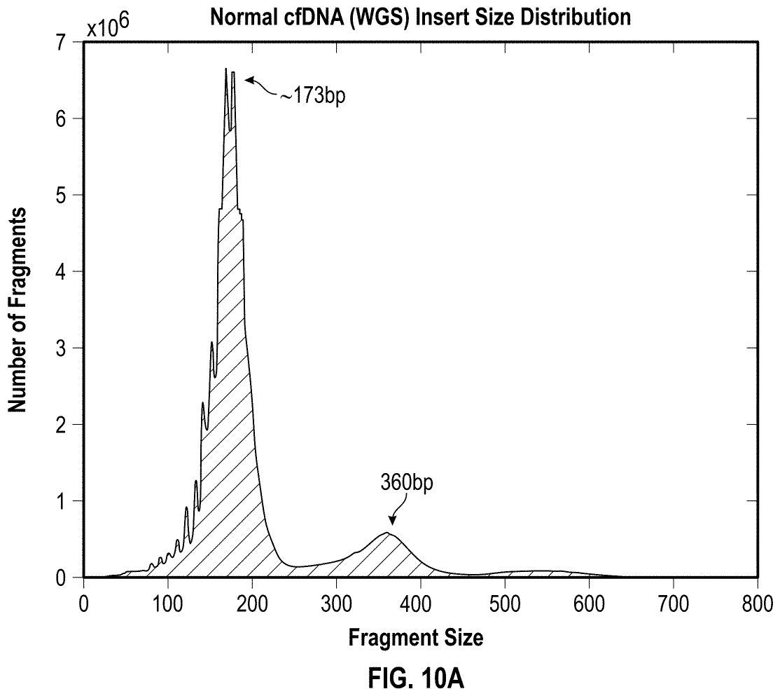

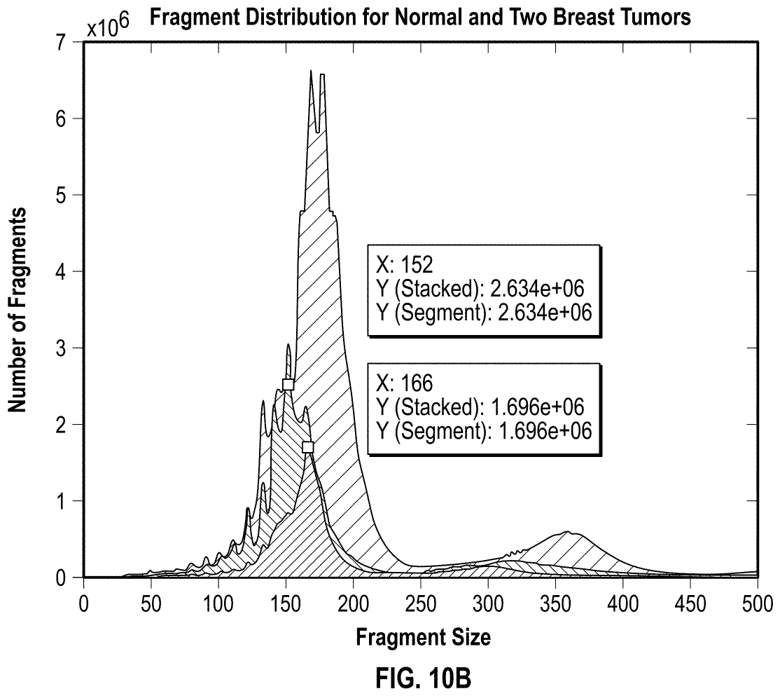

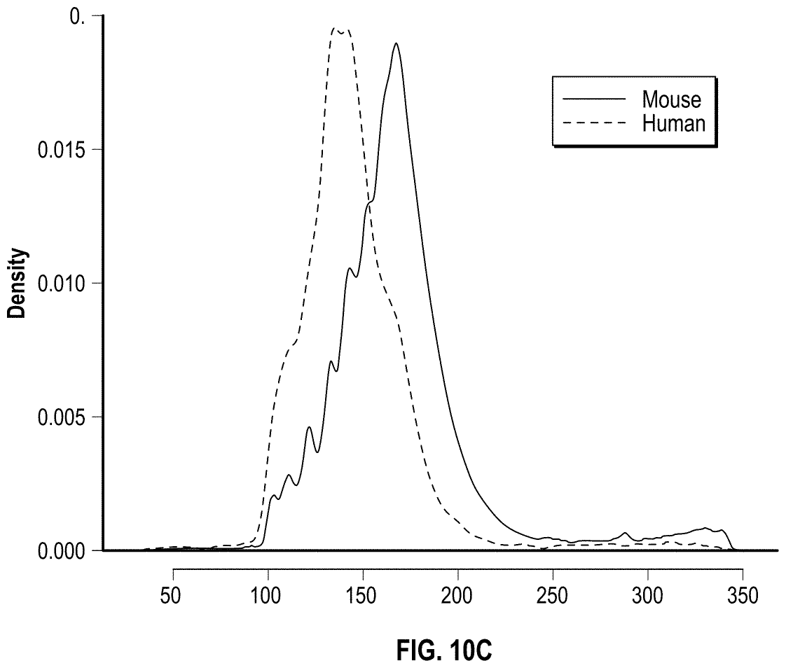

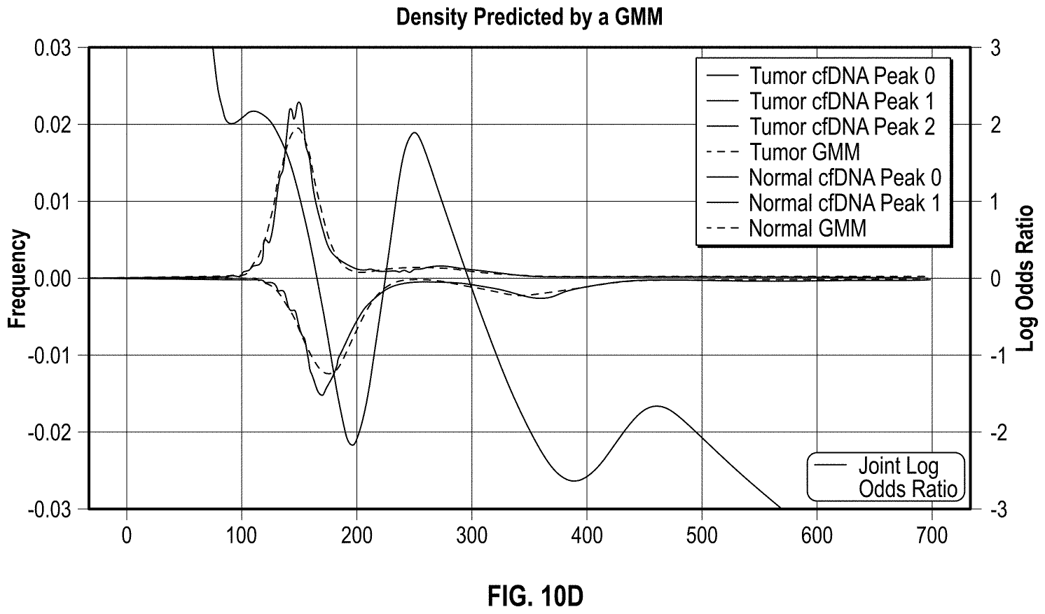

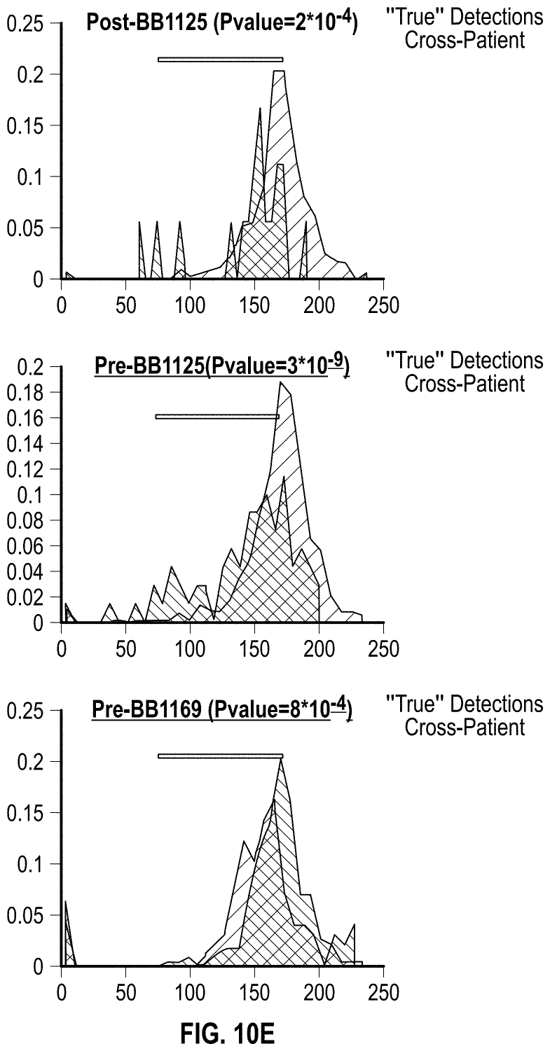

cfDNA fragment distribution have a unique profile due to the DNA degradation during blood circulation. Healthy normal cfDNA sample show the fragment size distribution shown in FIG. 10A. Circulating DNA fragments that originate from the tumor show shorter fragment size in comparison to “normal” DNA fragments that originate mainly from apoptosis of hematopoietic cells (immune cells). Breast tumor cfDNA (red and purple) show a fragment size shift compared to normal cfDNA sample (FIG. 10B). Calculating the center-of-mass (COM) of the first nucleosome (the peak around 170 bp) show a shift to lower COM that correspond linearly to the TF. Using human tumor xenograft models (PDX) in mice show that circulating DNA that is from the tumor origin (red, aligned to human) is significantly shorter than circulating DNA that is from normal origin (black, aligned to mouse). See FIG. 10C.

To generate a robust model that can quantify the probability of a single DNA fragment to be from tumor or normal origin we used a joint gaussian mixture model (GMM) to characterize the fragment size distribution of circulating DNA. Circulating tumor DNA model (red dashed line) was estimated by applying the GMM analysis to circulating tumor DNA extracted from our PDX samples, using only circulating DNA that is aligned to the human genome. Circulating normal DNA model (gray dashed line) was estimated by applying the GMM analysis to circulating DNA from plasma samples of healthy human volunteers. The joint log odds ratio (yellow line) was then used to estimate the probability of a fragment size of a specific circulating DNA to be from tumor or normal origin. Data are shown in FIG. 10D.

Patient specific mutation detections can be used to check if these DNA fragments correspond with tumor origin based on their fragment size distribution and the GMM joint log odds ratio. To increase confidence and decrease batch effect bias, an intra-patient control was developed using the cross-patient detection. For example, in the specific patient shown below the detected tumor mutation (gray, matched detections) are in and show tendency for a fragment size shift towards low fragment size. On the same patient sample, mutations that are associated with other patients were detected (red, cross-patient detection), these artefactual detections share the same Tobacco signature context-information patterns but are not true detection. Interestingly these cross-patient detections do not show the tendency for low fragment size shift, and their fragment size distribution is significantly different from the true tumor detections (Wilcoxon rank-sum, Pvalue 3*10⁻⁹). Using the GMM joint log odds ratio confirms that the patient specific mutation detection is from tumor origin (joint log odds ratio=0.3) while the artefactual mutations from the same patient sample are coming from normal origin (joint log odds ratio=−0.35). Representative data for three patients are shown in FIG. 10E.

### Example 3B: Orthogonal Integration of Fragment Size in the Context of CNV Markers

cfDNA fragment distribution have a unique profile due to the DNA degradation during blood circulation. Healthy normal cfDNA sample show a variation in the distribution of the fragment sizes (see, above, FIG. 10A and FIG. 10B). Here, in the context of analyzing center-of-mass (COM) distributions, calculation of the COM of first nucleosome (the peak around 170 bp) indicates a shift to lower COM that correspond linearly to the TF.

Comparative analysis of fragment size center-of-mass (COM) between patients may be limited with respect to sensitivity and may also be prone to batch effects. Intra-patient local fragment size COM can change due to epigenetic signatures or due to copy-number-events. Indeed, in amplification segments there is a local increase in tumor fraction (due to the increase in the proportion of tumor DNA) and therefore decrease in the local fragment size center-of-mass (COM). On the other end, in deletion segments there is a local decrease in tumor fraction (due to the decrease in the proportion of tumor DNA) and therefore increase in the local fragment size center-of-mass (COM).

Validating this concept on a plasma sample from a cancer patient, a clear negative correlation between the log 2 of the depth coverage (log 2>0.5=amplification, log 2<−0.5=deletion) and the local fragment size center-of-mass (COM) in that segment was identified. See, FIG. 11B. Further validation across plasma samples from 12 different cancer patients show a clear relationship between depth coverage based CNV detection and fragment size center-of-mass (COM) based CNV detection (FIG. 11C), which relationship does not evident in normal (healthy) plasma samples (FIG. 11D).

Multiple quantitative features can be extracted from this depth coverage (Log 2) and fragment size (COM) relationship per sample. More specifically, center-of-mass of the neutral regions (Log 2=0), slope of Log 2/COM relationship, and R² of Log 2/COM relationship. These features show a dynamic response to changes in the patient tumor fraction after surgery or during therapy, e.g., below is a cancer patient that is progressing during therapy showing a decrease in COM and increase in absolute slope value and in R² (FIG. 11E and FIG. 11F). Changes can be distinguished even in minute amounts of tumor DNA, e.g., a second patient during therapy.

Using a multiple linear regression or GLM allows conversion of the log 2/COM features to tumor fraction in order to monitor patients post-surgery and during treatment (FIG. 11G). For instance, outcomes of patients undergoing therapy were monitored over a 6 week (42-days) period. The estimated tumor fractions (FIG. 11I) and normalized CNV scores (FIG. 11J) were tabulated and presented in comparative bar charts for residual disease monitoring. The data show that patient 4, but not patients 1-3, responded to treatment over time, as evidenced by the fact that eTF for this patient at 42 days post-treatment with the drug was markedly lower compared to eTF at the time of therapy (FIG. 11I). Analysis of normalized CNV scores also leads to similar conclusions, vis-à-vis, a positive response in patient 4 who is undergoing a combination of immunotherapy and chemotherapy, which contrasts with patients 1-3 undergoing monotherapy (either chemotherapy or immunotherapy alone). The treatment response outcome was confirmed by imaging and long term clinical follow-up and was shown to be concordant with the eTF predictions.

### Example 4: Sensitive ctDNA Detection Using Genome-Wide Integration of Large Somatic Copy Number Variations (sCNVs)

In addition to somatic point mutations, cancer genomes are characterized by substantial aneuploidy. Through this process, large swaths of the genome undergo amplifications and deletions, yielding potentially robust signals for ctDNA detection. This is mainly because the WGS coverage depth is a function of the DNA content at each site. Other prominent examples include the shorter fragment length of ctDNA compared with normal cfDNA and nucleosome positioning information.

Thus, WGS offers the added advantage over targeted sequencing due to the abundance of orthogonal information sources to increase detection. To leverage this orthogonal genome-wide signal provided by WGS, a similar approach was developed to utilize differential read depth coverage in large amplification and deletion genomic segments. This read depth detection method is designed to integrate millions of small genomic windows in order to sensitively detect minute depth changes in areas of patient specific sCNV, thus allowing sensitive discrimination between low TF plasma and healthy (TF=0) controls.

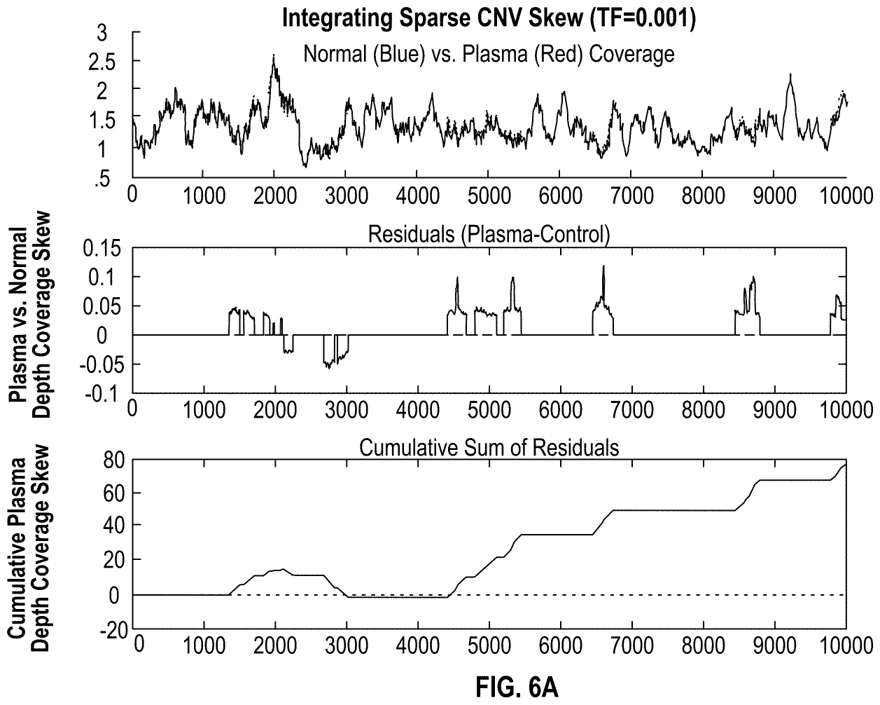

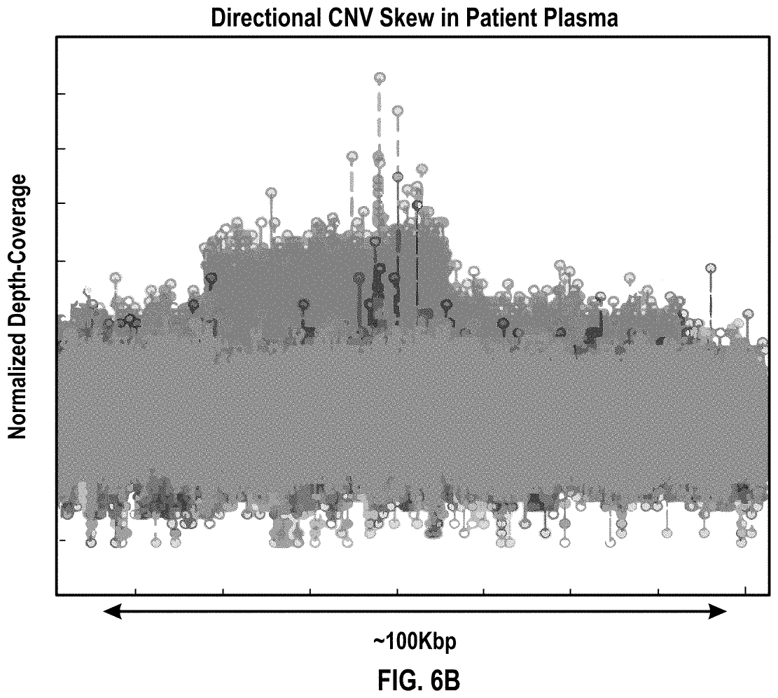

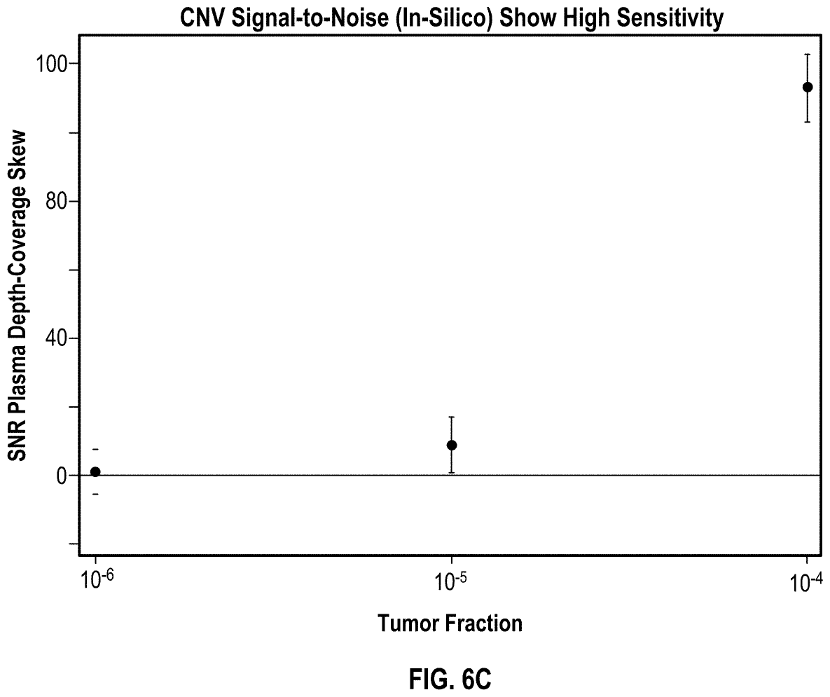

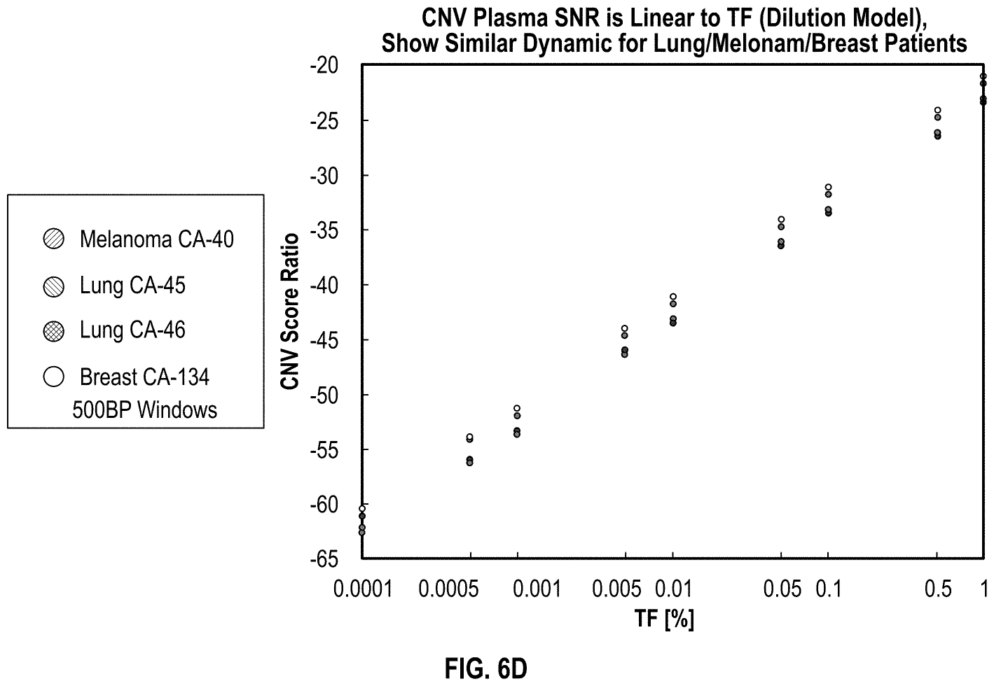

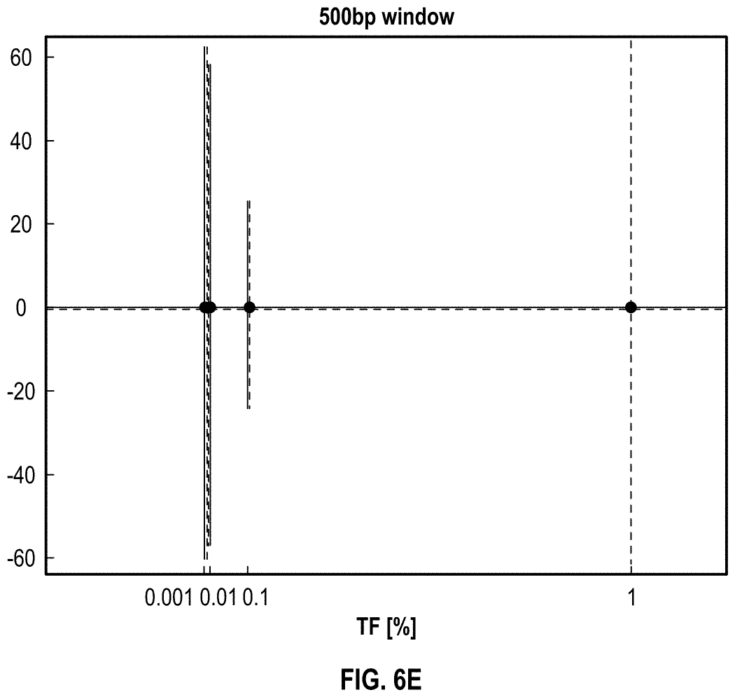

The disclosure therefore provides an analytic approach to integrate large number of directional depth coverage skews across large genomic CNV segments (FIG. 6A). Testing this on our NSCLC virtual plasma samples, a high detection sensitivity down to TF 1/100,000 was achieved via the integration of genome-wide CNV pattern (FIG. 6B). Moreover, comparing between the detected signal and TF show a linear (R²=1, P value=2*10⁻²⁴) relationship, indicating an adequate modeling by a simple dilution model, where the tumor local depth-coverage difference (amplification, deletion) is diluted by the proportional mixing with normal reads. This clear relationship enables the calculation of TF from the empirical patient measurements. This approach as well as the SNV approach will be validated side-by-side on the same patient cohort described above and will serve to build a joint classification model to synergistically improve sensitivity by integrating these orthogonal signals.

It is to be noted that the instant method provides complementary sensitive detection for patients with low SNV mutation load but high CNV load. Alternately, the methods described herein can be integrated with the SNV based method to further improve the detection independently of cfDNA abundance. Integration of the two methods on illustrative samples show potential detection of minimal residual disease. The data demonstrate that genome-wide sSNV integration offers sensitive MRD detection through the application of mutational inference signatures, even in the absence of a matched tumor sample.

The methods of the disclosure are not limited to the types of markers exemplified herein. For instance, residual disease detection/diagnosis may be performed by analyzing insertion or deletions (indels) in the genomic compendium of reads in a manner similar to SNV analysis (exemplified above in Example 2). Similarly, residual disease detection/diagnosis may be performed by analyzing structural variants (SV) in the genomic compendium of reads in a manner similar to CNV analysis (exemplified above in Example 3).

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

### Example 5: Comparative Assessment

The systems and methods of the disclosure were compared to art-known callers.

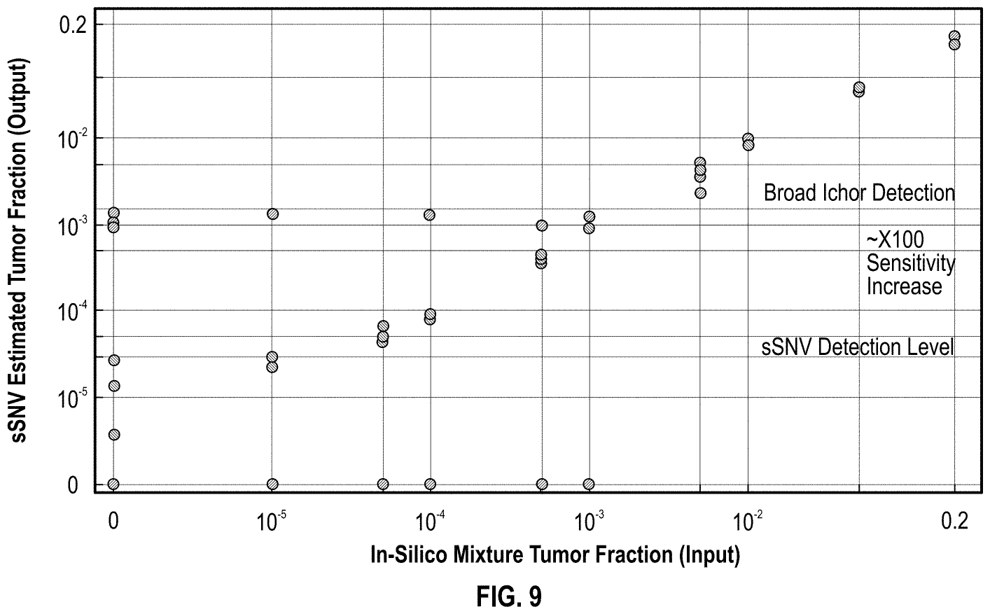

Current mutation callers do not work in low-TF regime. More, specifically MUTECT does not work below 1% TF. Applicable alternative methods for identifying ctDNA markers include high-coverage targeted sequencing with error-suppression (e.g., duplex sequencing). An example of an art method is given in Phallen et al. entitled “Direct Detection of Early Stage Cancers Using Circulating Tumor DNA” (Science Translational Medicine, 9, 203, 2017). The methods described in Phallen and other publications have limited sensitivity in low-TF (i.e., there is little to no detection below 1/1000 TF). A second art method from the Broad institute (called ICHOR) has similar limitations. ICHOR (see, Adalsteinsson et al. “Scalable whole-exome sequencing of cell-free DNA reveals high concordance with metastatic tumors,” Nature communications 8.1, 1324, 2017) attempts to integrate CNV information across WGS; however, the ICHOR method is completely different in approach than the present method. As can be seen from the comparative results presented in FIG. 9, the Broad ICHOR method has significantly lower sensitivity when compared to the present methods. Particularly, the 100-fold increase in sensitivity attained with the methods and systems of the disclosure are significantly superior and unexpectedly advantageous over ICHOR method.

The disclosure therefore relates to the following non-limiting embodiments:
Embodiment 1. A method for detecting residual disease in a subject in need thereof, comprising, (A) receiving a subject-specific genome wide compendium of genetic markers from a plurality of genetic markers from a first biological sample of a subject, the biological sample comprising a tumor sample and optionally a normal cell sample, wherein the compendium of genetic markers is selected from the group consisting of single nucleotide variation (SNV), short insertions and deletions (Indels), copy number variation, structural variants (SV) and combinations thereof; (B) detecting the subject-specific genome wide compendium of genetic markers in a second biological sample of the subject to generate a tumor-associated genome-wide representation of genetic markers in the second sample; (C) filtering artefactual noise markers from the genome-wide compendium of markers in the first and second biological samples, wherein the filtering comprises (a) statistically classifying each SNV or Indel in the compendium as signal or noise on the basis of probability of detection of noise (P_{N}) as a function of 1) mapping-quality (MQ) of a read group comprising the SNV, 2) fragment size length of a read group comprising the SNV, 3) consensus test within read duplicate families that comprises the SNV or Indel, and/or 4) base-quality (BQ) of the SNV or Indel; and/or (b) by statistically classifying each CNV or SV window in the compendium as signal or noise on the basis of 1) position thereof relative to the centromere, 2) mapping-quality (MQ) of the read group comprising a CNV or SV window, and/or 3) overlap with a cfDNA mask (blacklist); (D) computing an estimated tumor fraction (eTF) of the first and second biological sample on the basis of one or more integrative mathematical models; and (E) detecting a residual disease in the subject if the estimated tumor fraction exceeds an empirical threshold calculated using a background noise model.
Embodiment 2. The method according to Embodiment 1, wherein, step (A) comprises receiving a subject-specific genome wide compendium of genetic markers from a plurality of genetic markers from a biological sample comprises a tumor sample of a subject and a normal cell sample.
Embodiment 3. The method according to any one of Embodiments 1 and 2, wherein the read group comprises a set of reads that cover a specific SNV or indel site, or a set of reads that are included in a specific CNV or SV genomic window.
Embodiment 4. The method according to any one of Embodiments 1 to 3, wherein the tumor sample comprises a resected tumor or FNA, including snap frozen tissue, OCT embedded tissue or FFPE.
Embodiment 5. The method according to any one of Embodiments 1 to 4, wherein the normal sample comprises peripheral blood mononuclear cells (PMBC), or saliva or skin sample.
Embodiment 6. The method according to any one of Embodiments 1 to 5, wherein the plurality of genetic markers are received by whole-genome sequencing the subject's biological sample.
Embodiment 7. The method according to any one of Embodiments 1 to 6, wherein the compendium of genetic markers from the plurality of genetic markers from the first biological sample of the subject comprises high mutation rate and/or high number of CNVs or SVs.
Embodiment 8. The method according to Embodiment 7, wherein the high mutation rate comprises a mutation rate of at least 1 somatic single nucleotide polymorphism or indel per mega base pair and wherein a high copy number variation comprises somatic CNVs or SVs of at least 5 mega base pair in cumulative size.
Embodiment 9. The method according to any one of Embodiments 1 to 8, wherein the background noise model comprises measuring the error rate of detection in normal healthy samples and translating the error rate to basal noise eTF estimation model.
Embodiment 10. The method according to Embodiment 9, wherein a threshold calculated by eTF estimation model is between 10⁻⁴ to 10⁻⁶.
Embodiment 11. The method according to any one of Embodiments 1 to 11, wherein step (A) comprises receiving a subject-specific genome wide compendium of somatic genetic markers from a plurality of genetic markers from a biological sample of a subject, the biological sample comprising a tumor sample and a normal cell sample; and step (B) comprises subsequently detecting the subject-specific genome wide compendium of genetic markers in the second biological sample comprising plasma sample of the subject to generate a temporally updated tumor-associated genome-wide representation of the genetic markers in the patient plasma.
Embodiment 12. The method according to any one of Embodiments 1 to 11, wherein the normal cell sample comprises PMBC, saliva sample, hair sample, or skin sample.
Embodiment 13. The method according to any one of Embodiments 1 to 12, wherein the subject is a human and the subject's second biological sample is a biological material selected from the group consisting of blood, cerebral spinal fluid, pleural fluid, ocular fluid, stool, urine, and a combination thereof.
Embodiment 14. A method for quantitative estimation of the patient minimal residual disease burden during patient therapy, during patient observation or during a follow up period, comprising implementing (A) receiving a subject-specific genome wide compendium of genetic markers from a plurality of genetic markers from a first biological sample of a subject, the biological sample comprising a tumor sample and optionally a normal cell sample, wherein the compendium of genetic markers is selected from the group consisting of single nucleotide variation (SNV), short insertions and deletions (Indels), copy number variation, structural variants (SV) and combinations thereof; (B) detecting the subject-specific genome wide compendium of genetic markers in a second biological sample of the subject to generate a tumor-associated genome-wide representation of genetic markers in the second sample; (C) filtering artefactual noise markers from the genome-wide compendium of markers in the first and second biological samples, wherein the filtering comprises (a) statistically classifying each SNV or Indel in the compendium as signal or noise on the basis of probability of detection of noise (P_{N}) as a function of 1) mapping-quality (MQ) of a read group comprising the SNV, 2) fragment size length of a read group comprising the SNV, 3) consensus test within read duplicate families that comprises the SNV or Indel, and/or 4) base-quality (BQ) of the SNV or Indel; and/or (b) by statistically classifying each CNV or SV window in the compendium as signal or noise on the basis of 1) position thereof relative to the centromere, 2) mapping-quality (MQ) of the read group comprising a CNV or SV window, and/or 3) overlap with a cfDNA mask (blacklist); (D) computing an estimated tumor fraction (eTF) of the first and second biological sample on the basis of one or more integrative mathematical models; and (E) detecting a residual disease in the subject if the estimated tumor fraction exceeds an empirical threshold calculated using a background noise model.
Embodiment 15. The method according to Embodiment 14, wherein the (E) further comprises detection a residual disease in the subject after resective surgery; detection of residual disease during or after therapy; detection of residual disease to monitor effectiveness of therapy; detection of residual disease to monitor recurrent or relapse of cancer; or a combination thereof.
Embodiment 16. The method according to Embodiment 15, wherein resective surgery comprises lymph node biopsy; head or neck surgery; uterus or endometrial biopsy; bladder biopsy; mastectomy; prostatectomy; skin lesion removal; small bowel resection; gastrectomy; thoracotomy; adrenalectomy; colectomy; oophorectomy; thyroidectomy; hysterectomy; glossectomy; or colon polypectomy.
Embodiment 17. The method according to Embodiment 15, wherein the therapy comprises chemotherapy, immunotherapy, targeted therapy, radiation therapy or a combination thereof.
Embodiment 18. The method according to any one of Embodiments 14 to 17, wherein the BQ, MQ and fragment size parameters of the marker are optimized using an ROC curve.
Embodiment 19. The method according to any one of Embodiments 14 to 18, comprising employing a combined base quality mapping quality (BQ MQ) parameter.
Embodiment 20. The method according to any one of Embodiments 14 to 19, further comprising receiving a plurality of genetic markers from a biological sample of a subject, the biological sample comprising a tumor sample and a normal cell sample, and generating a subject-specific genome wide compendium of genetic markers from the received plurality of genetic markers.
Embodiment 21. The method according to any one of Embodiments 14 to 20, further comprising detecting the subject-specific genome wide compendium of genetic markers in a third biological sample of the subject to compare to the subject-specific genome wide compendium of genetic markers generated in the subject's first biological sample.
Embodiment 22. The method according to Embodiment 21, wherein the third biological sample is a plasma sample of the subject obtained to generate a temporally updated representation of tumor genome-wide genetic markers in the patient plasma.
Embodiment 23. The method according to any one of Embodiments 14 to 22, further comprising empirically determining a background noise threshold, wherein a tumor fraction above the background noise threshold provides a quantitative estimation of tumor burden.
Embodiment 24. The method according to any one of Embodiments 14 to 23, wherein a tumor fraction below the noise threshold is considered non-detected (N.D.).
Embodiment 25. The method according to any one of Embodiments 14 to 24, wherein detecting comprises quantitative monitoring over time.
Embodiment 26. The method according to any one of Embodiments 14 to 25, wherein the tumor is brain cancer, lung cancer, skin cancer, nose cancer, throat cancer, liver cancer, bone cancer, lymphomas, pancreatic cancer, skin cancer, bowel cancer, rectal cancer, thyroid cancer, bladder cancer, kidney cancer, mouth cancer, stomach cancer, melanoma, osteosarcoma or solid state tumor which is heterogeneous or homogeneous in nature.
Embodiment 27. The method according to any one of Embodiments 14 to 26, wherein the tumor is tumor is lung adenocarcinoma, ductal adenocarcinoma, non-small-cell lung carcinoma lung adenocarcinoma (NSCLC LUAD), cutaneous melanoma, urothelial carcinoma or osteosarcoma.
Embodiment 28. The method according to any one of Embodiments 14 to 27, wherein the computing step further comprises: computing an eTF for SNV or indel markers by integrating a probabilistic model, wherein the probabilistic model comprises 1) integrated signal of plasma SNV or indel detection, 2) process-quality metrics comprising estimated genomic coverage and sequencing noise model, and/or 3) patient specific parameters comprising mutation load (N); and/or computing an eTF for CNV or SV markers by utilizing a probabilistic dilution model, wherein the probabilistic dilution model comprises 1) integrating directional depth of coverage skewed between plasma and normal patient samples in concordance with tumor CNV or SV directionality wherein amplification of copy number is skewed positively and deletion of copy number is skewed negatively; 2) integrating the cumulative depth of coverage skewed between tumor and normal (PBMC) patient samples; and/or 3) finding the dilution ratio between the above signals.
Embodiment 29. A system for detecting residual disease in a subject in need thereof, comprising, (A) an analyzing unit configured and arranged to filter artefactual noise markers from a genome-wide compendium of markers, wherein the genome-wide compendium of markers is generated from a plurality of genetic markers from a biological sample of a subject, the biological sample comprising a tumor sample and a normal cell sample, wherein the compendium of genetic markers is selected from the group consisting of single nucleotide variation (SNV), indels, copy number variation, SV and combinations thereof, the analyzing unit further comprising detecting the subject-specific genome wide compendium of genetic markers in a second biological sample to generate a representation of tumor genome-wide genetic markers in the second sample, the analyzing unit further comprising a classification engine, wherein the classification engine: (a) statistically classifies each SNV in the compendium as signal or noise on the basis of probability of detection of noise (P_{N}) as a function of 1) mapping-quality (MQ) of the read group comprises the SNV or Indel, 2) fragment size length of the read group comprises the SNV or Indel, 3) consensus test within read duplicate families that comprises the specific SNV, 4) base-quality (BQ) of the SNV or Indel, and/or (b) statistically classifies each CNV or SV window in the compendium as signal or noise on the basis of—1) position thereof relative to the centromere, 2) mapping-quality (MQ) of the read group comprises the CNV or SV window, 3) Representation of the CNV or SV window in cfDNA data; (B) a computing unit configured and arranged to calculate estimated tumor fraction (eTF) of the sample on the basis of one or more integrative mathematical models; and (C) a display unit that outputs a residual disease profile of the subject based on the estimated tumor fraction, wherein a residual disease in the subject is output in the residual disease profile if the estimated tumor fraction exceeds an empirical threshold calculated by a background noise model.
Embodiment 30. The system or method according to any one of the foregoing embodiments, wherein the computing unit is further configured and arranged to: compute an eTF for SNV or Indel markers by integrating a probabilistic model, wherein the probabilistic model comprises 1) integrated signal of plasma SNV or Indel detection, 2) process-quality metrics comprising estimated genomic coverage and sequencing noise model, and/or 3) patient specific parameters comprising mutation load (N); and/or computing an eTF for CNV or SV markers by utilizing a probabilistic mixture model, wherein the probabilistic dilution model comprises 1) integrating directional depth of coverage skewed between plasma and normal patient samples in concordance with tumor CNV or SV directionality wherein amplification of copy number is skewed positively and deletion of copy number is skewed negatively; 2) integrating the cumulative depth of coverage skewed between tumor and normal patient samples; and/or 3) finding a dilution ratio between the above signals.
Embodiment 31. The system or method according to Embodiment 30, wherein, the computing unit (B) comprises a processor, the processor configured to execute the computer-readable instructions, which when executed, estimates tumor fraction (eTF) of the sample on the basis of one or more of the following integrative mathematical models (1) eTF[SNV]=1−[1−(M−E(σ)*R)/N]\hat{ }(1/cov), wherein M is the number of tumor-specific SNV compendium detections in the patient plasma sample, a is a measure of empirically-estimated error-rate, R is the total number of unique reads in the SNV compendium region of interest (ROI), N is tumor mutation load, and cov is the average number of unique reads per site in the SNV compendium ROI; and/or (2) eTF [CNV]=(sum_{i}[(P(i)−N(i))*sign[T(i)−N(i)]]−E(sigma))/(sum_{i}[abs(T(i)−N(i))]−E(6)), wherein P is a median depth-coverage value in a genomic window indexed by {i} representing plasma depth coverage, normalized by either robust-zscore method or robust PCA compared to a cohort of normal samples; T is a median depth value in a genomic window indexed by {i} representing tumor depth coverage, normalized by either robust-zscore method or robust PCA compared to a cohort of normal samples; N is a median depth value in a genomic window indexed by {i} representing normal depth coverage, normalized by either robust-zscore method or robust PCA compared to a cohort of normal samples; and {i} is a discrete index counting all the genomic windows that cover the patient tumor-specific amplification and deletion genomic segments.
Embodiment 32. A computer readable medium comprising computer-executable instructions, which, when executed by a processor, cause the processor to carry out a method or a set of steps for detection of residual disease, the method or steps comprising, (A) receiving a subject-specific genome wide compendium of genetic markers from a plurality of genetic markers from a biological sample of a subject, the biological sample comprising a tumor sample and optionally a normal cell sample, wherein the compendium of genetic markers is selected from the group consisting of single nucleotide variation (SNV), short insertions and deletions (Indels), copy number variation, structural variants (SV) and combinations thereof; (B) detecting the subject-specific genome wide compendium of genetic markers in a second biological sample of the subject to generate a tumor-associated genome-wide representation of genetic markers in the second sample; (C) filtering artefactual noise markers from the genome-wide compendium of markers by statistically classifying each SNV or Indel in the compendium as signal or noise on the basis of probability of detection of noise (P_{N}) as a function of 1) mapping-quality (MQ) of a read group comprising the SNV, 2) fragment size length of a read group comprising the SNV, 3) consensus test within read duplicate families that comprises the SNV or Indel, 4) base-quality (BQ) of the SNV or Indel; and/or by statistically classifying each CNV or SV window in the compendium as signal or noise on the basis of 1) position thereof relative to the centromere, 2) mapping-quality (MQ) of the read group comprising a CNV or SV window, 3) overlap with a cfDNA mask (blacklist); (D) computing an estimated tumor fraction (eTF) of the biological sample on the basis of one or more integrative mathematical models; and (E) diagnosing a residual disease in the subject based on the estimated tumor fraction and an empirical threshold calculated by background noise model.
Embodiment 33. A method for detecting minimal residual disease in a subject, comprising (A) receiving a genome-wide compendium of reads in genetic data sequenced from a plurality of biological samples received from the subject, the plurality of biological samples comprising a tumor sample, a normal sample and a plasma sample; (B) performing mutation calling on tumor and peripheral blood mononuclear cells (PBMC) samples from the subject, wherein the mutation calling comprises MUTECT, LOFREQ and/or STRELKA mutation calling to generate subject-specific reads of somatic SNV (sSNV) or indels as a personalized reference set; (C) collecting and filtering the reads from the subject-specific somatic SNV (sSNV) or indels, the collecting and filtering comprising (1) removing low mapping quality reads (e.g., <29, ROC optimized); (2) building duplication families (represent multiple PCR/sequencing copies of the same DNA fragment) and producing corrected read based on a consensus test; (3) removing low base quality reads (e.g., <21, ROC optimized); and (4) removing high fragment size reads (e.g., >160, ROC optimized); (D) computing the number of subject-specific mutation sites that have at least one supporting read (in the filtered set) with the exact same substitution as in the tumor; (F) estimating a tumor fraction for SNV based on the mathematical model eTF[SNV]=1−[1−(M−E(σ)*R)/N]\hat{ }(1/cov) . . . . (Equation 1), wherein M is the number of tumor-specific compendium detections in the patient sample, a is a measure of empirically-estimated noise, R is the total number of unique reads in a region of interest (ROI), N is tumor mutation load, and cov is the average number of unique reads per site in the ROI; (G) comparing eTF[SNV] against a detection threshold which comprises an empirically measured basal noise TF estimation from healthy samples, wherein an eTF[SNV] that is above a threshold level (e.g., 2 standard deviations of the noise TF distribution (FPR<2.5%)) is indicative of positive detection; and (K) detecting the residual disease in the subject based on the eTF estimation exceeding the detection threshold level.
Embodiment 34. A method for detecting a minimal residual disease in a subject, comprising (A) receiving a genome-wide compendium of reads in genetic data sequenced from a plurality of biological samples received from the subject, the plurality of biological samples comprising a tumor sample, a normal sample and a plasma sample; (B) performing CNV or SV calling on tumor and peripheral blood mononuclear cells (PBMC) samples from the subject, generating a reference segmentation of a plurality of CNV or SV segments or SV which exceed a threshold length (e.g., >2 Mbp, preferably >5 Mbp), and annotating a directionality of the segment, wherein amplification is annotated positively and deletion is annotated negatively; (C) collecting single-bp depth coverage information for the plasma, tumor and PBMC samples covering a patient specific CNV or SV segmentation region of interest (ROI); (D) dividing the patient specific CNV or SV segmentation ROI to 500 bp windows and calculating a median value per window (artifact suppression) for all samples and window; (E) generating normalized depth coverage information for all 500 bp windows using (a) Robust zscore normalization per sample; and/or (2) Robust Principal Component Analysis (RPCA); (F) filtering windows from the patient-specific segmentation, wherein filtration comprises: (1) removing low mapping quality reads (e.g., <29, ROC optimized); and/or (2) removing centromere regions (e.g., removing windows with normalized normal value above 10); and/or (3) removing non-represented regions in cfDNA (e.g., removing windows that are not included in a cfDNA representation mask composed from multiple cfDNA samples); (G) Integrating directional depth of coverage skewed between plasma and normal (PBMC) patient samples using the mathematical model sumᵢ[(P(i)−N(i))*sign[T(i)−N(i)]]−E(σ) . . . . (Equation 2), wherein P is a median depth-coverage value in a genomic window indexed by {i} representing plasma depth coverage, normalized by either robust-zscore method or robust PCA compared to a cohort of normal samples; E(sigma) is a measure of empirically-estimated error-rate; T is a median depth value in a genomic window indexed by {i} representing tumor depth coverage, normalized by either robust-zscore method or robust PCA compared to a cohort of normal samples; and N is a median depth value in a genomic window indexed by {i} representing normal depth coverage, normalized by either robust-zscore method or robust PCA compared to a cohort of normal samples; (H) integrating the cumulative depth of coverage skewed between tumor and normal (PBMC) patient samples using the mathematical model sumᵢ[abs(T(i)−N(i))]−E(σ)) . . . . (Equation 3), wherein T, N and E(σ) are as provided above; (I) calculating a dilution ratio between directional depth coverage of (G) and cumulative depth coverage (H) which corresponds to the estimated tumor fraction for CNV or SV (eTF[CNV])=(sumᵢ[(P(i)−N(i))*sign[T(i)−N(i)]]−E(σ))/(sumᵢ[abs(T(i)−N(i))]−E(σ)) . . . . (Equation 4); (J) comparing eTF[CNV] against a detection threshold which comprises an empirically measured basal noise TF estimation from healthy samples, wherein an eTF[CNV] that is above a threshold level (e.g., 2 standard deviations of the noise TF distribution (FPR<2.5%)) is indicative of positive detection; and (K) detecting the residual disease in the subject based on the eTF estimation exceeding the detection threshold level.
Embodiment 35. A method for detecting residual disease in a subject in need thereof, comprising, (A) receiving a first subject-specific genome wide compendium of reads associated with genetic markers from a first biological sample of a subject, the first biological sample comprising a baseline sample and a normal cell sample, wherein the first compendium of reads each comprise reads of a single base pair length and wherein the baseline sample comprises a tumor sample or a plasma sample; (B) filtering artefactual sites from the first compendium of reads, wherein the filtering comprises removing, from the first compendium of genetic markers, recurring sites generated over a cohort of reference healthy samples, and/or identifying germ line mutations in peripheral blood mononuclear cells of the normal cell sample and removing said germ line mutations from the from the first compendium of genetic markers; (C) detecting reads from a second subject-specific genome wide compendium of genetic markers in a second biological sample of the subject to generate a tumor-associated genome-wide representation of genetic markers in the second sample; (D) filtering noise from the first and second genome-wide compendium of reads using at least one error suppression protocol to produce a first filtered read set for the first genome-wide compendium of reads and a second filtered read set for the second genome-wide compendium of reads, wherein the at least one error suppression protocol comprises (a) calculating the probability that any single nucleotide variation in the first and second compendium is an artefactual mutation, and removing said mutation, wherein the probability is calculated as a function of features selected from the group consisting of mapping-quality (MQ), variant base-quality (MBQ), position-in-read (PIR), mean read base quality (MRBQ), and combinations thereof, and/or (b) removing artefactual mutations using discordance testing between independent replicates of the same DNA fragment generated from polymerase chain reaction or sequencing processing, and/or duplication consensus wherein artefactual mutations are identified and removed when lacking concordance across a majority of a given duplication family; (E) computing an estimated tumor fraction (eTF) of the first and second biological sample using the first and second filtered read sets by applying a background noise model to one or more integrative mathematical models; and (F) detecting a residual disease in the subject if the estimated tumor fraction in the second biological sample exceeds an empirical threshold.
Embodiment 36. A method for detecting residual disease in a subject in need thereof, comprising, (A) receiving a first subject-specific genome wide compendium of reads associated with genetic markers from a first biological sample of a subject, the first biological sample comprising a baseline sample, wherein the first compendium of reads each comprise a copy number variation (CNV) or structural variations (SVs) and wherein the baseline sample comprises a tumor sample or a plasma sample; (B) receiving a second subject-specific genome wide compendium of reads associated with genetic markers from a second biological sample of a subject, the second biological sample comprising a peripheral blood mononuclear cell sample (PBMC), wherein the second compendium of genetic markers each comprise CNVs or SVs; (C) filtering artefactual sites from the first and second compendium of reads, wherein the filtering comprises removing, from the first and second compendium of reads, recurring sites generated over a cohort of reference healthy samples; identifying shared CNVs/SVs between the first and second compendium as germ line mutations and removing said mutations from the first and second compendium of reads; (D) detecting reads from a third subject-specific genome wide compendium of genetic markers in a third biological sample of the subject to generate a tumor-associated genome-wide representation of genetic markers in the third sample; (E) normalizing each of the first, second and third compendium of reads to produce a first filtered read set for the first genome-wide compendium of reads, a second filtered read set for the second genome-wide compendium of reads, and a third filtered read set for the third genome-wide compendium of reads; (F) computing an estimated tumor fraction (eTF) of the third biological samples, using the third filtered read set, by applying a background noise model to one or more integrative mathematical models, the one or more models producing a first eTF using the first filtered read set, and/or the one or more models producing a second eTF using the second filtered read set; and (G) detecting a residual disease in the subject if the estimated tumor fraction in the third biological sample exceeds an empirical threshold.
Embodiment 37. A system for detecting residual disease in a subject in need thereof, comprising, an analyzing unit, the analyzing unit comprising a pre-filter engine configured and arranged to receive a first subject-specific genome wide compendium of reads associated with genetic markers from a first biological sample of a subject, the first biological sample comprising a baseline sample and a normal sample, wherein the first compendium of reads each comprise reads of a single base pair length and wherein the baseline sample comprises a tumor sample or a plasma sample; and filter artefactual sites from the first compendium of reads, wherein the filtering comprises removing, from the first compendium of genetic markers, recurring sites generated over a cohort of reference healthy samples, and/or identifying germ line mutations in peripheral blood mononuclear cells of the normal cell sample and removing said germ line mutations from the from the first compendium of genetic markers; and a correction engine configured and arranged to receive reads from a second subject-specific genome wide compendium of genetic markers in a second biological sample of the subject to generate a tumor-associated genome-wide representation of genetic markers in the second sample; and filter noise from the first and second genome-wide compendium of reads using at least one error suppression protocol to produce a first filtered read set for the first genome-wide compendium of reads and a second filtered read set for the second genome-wide compendium of reads, wherein the at least one error suppression protocol comprises (a) calculating the probability that any single nucleotide variation in the first and second compendium is an artefactual mutation, and removing said mutation, wherein the probability is calculated as a function of features selected from the group consisting of mapping-quality (MQ), variant base-quality (MBQ), position-in-read (PIR), mean read base quality (MRBQ), and combinations thereof, and/or (b) removing artefactual mutations using discordance testing between independent replicates of the same DNA fragment generated from polymerase chain reaction or sequencing processing, and/or duplication consensus wherein artefactual mutations are identified and removed when lacking concordance across a majority of a given duplication family; and a computing unit configured and arranged to compute an estimated tumor fraction (eTF) of the first and second biological sample using the first and second filtered read sets by applying a background noise model to one or more integrative mathematical models; and detect a residual disease in the subject if the estimated tumor fraction in the second biological sample exceeds an empirical threshold.
Embodiment 38. A system for detecting residual disease in a subject in need thereof, comprising, a pre-filter engine configured and arranged to receive a first subject-specific genome wide compendium of reads associated with genetic markers from a first biological sample of a subject, the first biological sample comprising a baseline sample, wherein the first compendium of reads each comprise reads of a single base pair length and wherein the baseline sample comprises a tumor sample or a plasma sample; receive a second subject-specific genome wide compendium of reads associated with genetic markers from a second biological sample of a subject, the second biological sample comprising a peripheral blood mononuclear cell sample (PBMC), wherein the second compendium of genetic markers each comprise a copy number variation (CNV); and filter artefactual sites from the first and second compendium of reads, wherein the filtering comprises removing, from the first and second compendium of reads, recurring sites generated over a cohort of reference healthy samples; identifying shared CNVs between the first and second compendium as germ line mutations and removing said mutations from the first and second compendium of reads; and a correction engine configured and arranged to receive reads from a third subject-specific genome wide compendium of genetic markers in a second biological sample of the subject to generate a tumor-associated genome-wide representation of genetic markers in the third sample; and normalize each of the first, second and third compendium of reads to produce a first filtered read set for the first genome-wide compendium of reads, a second filtered read set for the second genome-wide compendium of reads, and a third filtered read set for the third genome-wide compendium of reads; and a computing unit configured and arranged to compute an estimated tumor fraction (eTF) of the third biological samples, using the third filtered read set, by applying a background noise model to one or more integrative mathematical models, the one or more models producing a first eTF using the first filtered read set, and/or the one or more models producing a second eTF using the second filtered read set; and detect a residual disease in the subject if the estimated tumor fraction in the third biological sample exceeds an empirical threshold.
Embodiment 39. The method of Embodiment 35, wherein the markers comprise single nucleotide variations (SNVs) or insertion/deletions (indels); preferably SNV.
Embodiment 40. The method of Embodiments 35 and 39 wherein filtering recurring sites generated over a cohort of reference healthy samples comprises generating a panel of normal (PON) blacklist or mask.
Embodiment 41. The method of any of Embodiments 35 and 39 to 40, wherein the normal sample comprises peripheral blood mononuclear cells (PBMC) and germ line mutations in PBMC are removed in the artefactual site filtration step (B).
Embodiment 42. The method of any of Embodiments 35 and 39 to 41, wherein in step (A), the first biological sample comprises plasma sample that is obtained from the subject pre-surgery or pre-therapy.
Embodiment 43. The method of any of Embodiments 35 and 39 to 42, wherein in step (C), the second biological sample comprises plasma sample which is obtained from the same subject post-therapy or post-surgery.
Embodiment 44. The method of any of Embodiments 35 and 39 to 43, wherein step (D) comprises employing a machine learning (ML) algorithm, e.g., deep convolutional neural network (CNN), recurrent neural network (RNN), random forest (RF), support vector machine (SVM), discriminant analysis, nearest neighbor analysis (KNN), ensemble classifier, or a combination thereof; preferably, support vector machine (SVM), to filter artefactual noise.
Embodiment 45. The method of any of Embodiments 35 and 39 to 44, wherein in step (D), the second error suppression step includes correction of artefactual mutations generated by PCR or sequencing using the comparison of independent replicates of the same original nucleic acid fragment.
Embodiment 46. The method of Embodiment 45, wherein in step (D), the second error suppression step includes correction of artefactual mutations generated by paired-end 150 bp sequencing, resulting in overlapping paired reads (R1 and R2), and discordance between R1 and R2 pairs are corrected back to the corresponding reference genome.
Embodiment 47. The method of any of Embodiments 35 and 39 to 46, wherein in step (D), the second error suppression step includes correction of duplication families generated during sequencing and/or PCR amplification, wherein the duplication families are recognized by 5′ and 3′ similarity as well as alignment position and wherein each duplication family is used to check the consensus of a specific mutation across independent replicates, thereby correcting artefactual mutations that do not show concordance in a majority of the duplication family.
Embodiment 48. The method of any of Embodiments 35 and 39 to 47, wherein in step (E), the mathematical model integrates a relationship between the coverage, mutation load, number of detected mutations and the tumor fraction (TF).
Embodiment 49. The method of any of Embodiments 35 and 39 to 48, wherein in step (E), the background noise calculation includes using patient specific mutation signature to calculate (1) the expected noise distribution over a cohort of healthy plasma samples (panel-of-normal or PON) or (2) the expected noise distribution across other patients (cross-patient analysis).
Embodiment 50. The method of Embodiment 49, wherein the background noise model provides an estimated mean and standard-deviation (μ, σ) of artefactual mutation detection rate.
Embodiment 51. The method of any of Embodiments 35 to 50, further comprising orthogonal integration of a secondary feature comprising fragment size shift.
Embodiment 52. The method of Embodiment 51, wherein intra-patient fragment size shifts in the list of tumor-specific markers and random markers are analyzed using statistical methods, e.g., tests for significance or Guassian mixture model (GMM).
Embodiment 53. The method of Embodiment 36, wherein the markers comprise copy number variations (CNVs).
Embodiment 54. The method of any one of Embodiments 36 and 37, wherein filtering recurring sites generated over a cohort of reference healthy samples comprises generating a panel of normal (PON) blacklist or mask.
Embodiment 55. The method of any of Embodiments 36 and 53 to 54, wherein germ line events in PBMC are removed in the artefactual site filtration step (C).
Embodiment 56. The method of any of Embodiments 36 and 53 to 55, wherein in step (A), the first biological sample comprises plasma sample that is obtained from the subject pre-surgery or pre-therapy and the second biological sample comprises PBMCs obtained from the same subject pre-surgery or pre-therapy.
Embodiment 57. The method of any of Embodiments 36 and 53 to 56, wherein in step (C), the third biological sample comprises plasma sample which is obtained from the same subject post-therapy or post-surgery.
Embodiment 58. The method of any of Embodiments 36 and 53 to 57, wherein in step (C) comprises binning (to ≥500 bp windows) a region-of-interest (ROI) containing all the genomic segments of the somatic tumor CNV (sT_CNV) and somatic PBMC CNV (sP_CNV); estimating the depth coverage (read count) in each window from a follow-up plasma sample; and calculating median depth coverage per window.
Embodiment 59. The method of any of Embodiments 36 and 53 to 58, wherein the follow-up plasma sample is obtained after surgery, during treatment, or at follow-up.
Embodiment 60. The method of any of Embodiments 36 and 53 to 59, wherein the normalization step includes normalizing depth coverage values to correct for GC-content and mappability biases by performing two LOESS regression curve-fitting on the bin-wise GC-fraction and mappability score.
Embodiment 61. The method of any of Embodiments 36 and 53 to 60, wherein the normalization step includes batch-effect correction using a robust-zscore normalization, which is applied to each sample separately.
Embodiment 62. The method of Embodiment 62, wherein the zscore normalization includes calculation of median and median-absolute-deviation (MAD) based on the neutral regions of each sample and normalizing all CNV bins are normalized by subtracting the median value and dividing the differential by MAD.
Embodiment 63. The method of any of Embodiments 36 and 53 to 62, wherein step (E) includes calculating depth coverage skew and/or fragment size center-of-mass (COM) skew in the third sample in comparison to a panel of normal (PON) healthy plasma samples.
Embodiment 64. The method of any of Embodiments 36 and 53 to 63, wherein step (E) includes calculation of tumor fraction by checking a linear dilution ratio between the cumulative signal detected at the follow-up plasma sample in comparison to the cumulative signal detected in the tumor sample.
Embodiment 65. The method of any of Embodiments 36 and 53 to 64, wherein in step (F), the background noise calculation includes using patient specific CNV/SV signature to calculate (1) the expected noise distribution over a cohort of healthy plasma samples (panel-of-normal or PON) or (2) the expected noise distribution across other patients (cross-patient analysis).
Embodiment 66. The method of Embodiment 65, wherein the background noise model provides an estimated mean and standard-deviation (μ, σ) of artefactual SNV/SV detection rate.
Embodiment 67. The method of any of Embodiments 36 and 53 to 66, further comprising orthogonal integration of a secondary feature comprising fragment size shift.
Embodiment 68. The method of Embodiment 67, wherein correlation between depth coverage skew and fragment size skew in CNV segments are analyzed to infer tumor fraction, e.g., using a generalized linear model (GLM).

For convenience, certain terms employed in the specification, examples and claims are collected here. Unless defined otherwise, all technical and scientific terms used in this disclosure have the same meanings as commonly understood by one of ordinary skill in the art to which this disclosure belongs.

Throughout this disclosure, various patents, patent applications and publications are referenced. The disclosures of these patents, patent applications, accessioned information (e.g., as identified by PUBMED, PUBCHEM, NCBI, UNIPROT, or EBI accession numbers) and publications in their entireties are incorporated into this disclosure by reference in order to more fully describe the state of the art as known to those skilled therein as of the date of this disclosure. This disclosure will govern in the instance that there is any inconsistency between the patents, patent applications and publications cited and this disclosure.

1. A method for detecting residual disease in a subject in need thereof,
comprising,
(A) receiving a first subject-specific genome wide compendium of reads associated with genetic markers from a first biological sample of a subject, the first biological sample comprising a baseline sample and a normal cell sample, wherein the first compendium of reads each comprise reads of a single base pair length and wherein the baseline sample comprises a tumor sample or a plasma sample;
(B) filtering artefactual sites from the first compendium of reads, wherein the filtering comprises removing, from the first compendium of genetic markers, recurring sites generated over a cohort of reference healthy samples, and/or identifying germ line mutations in peripheral blood mononuclear cells of the normal cell sample and removing said germ line mutations from the from the first compendium of genetic markers;
(C) detecting reads from a second subject-specific genome wide compendium of genetic markers in a second biological sample of the subject to generate a tumor-associated genome-wide representation of genetic markers in the second sample;
(D) filtering noise from the first and second genome-wide compendium of reads using at least one error suppression protocol to produce a first filtered read set for the first genome-wide compendium of reads and a second filtered read set for the second genome-wide compendium of reads, wherein the at least one error suppression protocol comprises (a) calculating the probability that any single nucleotide variation in the first and second compendium is an artefactual mutation, and removing said mutation, wherein the probability is calculated as a function of features selected from the group consisting of mapping-quality (MQ), variant base-quality (MBQ), position-in-read (PIR), mean read base quality (MRBQ), and combinations thereof; and/or (b) removing artefactual mutations using discordance testing between independent replicates of the same DNA fragment generated from polymerase chain reaction or sequencing processing, and/or duplication consensus wherein artefactual mutations are identified and removed when lacking concordance across a majority of a given duplication family;
(E) computing an estimated tumor fraction (eTF) of the first and second biological sample using the first and second filtered read sets by applying a background noise model to one or more integrative mathematical models;
and
(F) detecting a residual disease in the subject if the estimated tumor fraction in the second biological sample exceeds an empirical threshold.

2. A method for detecting residual disease in a subject in need thereof,
comprising,
(A) receiving a first subject-specific genome wide compendium of reads associated with genetic markers from a first biological sample of a subject, the first biological sample comprising a baseline sample, wherein the first compendium of reads each comprise a copy number variation (CNV) or structural variations (SVs) and wherein the baseline sample comprises a tumor sample or a plasma sample;
(B) receiving a second subject-specific genome wide compendium of reads associated with genetic markers from a second biological sample of a subject, the second biological sample comprising a peripheral blood mononuclear cell sample (PBMC), wherein the second compendium of genetic markers each comprise CNVs or SVs;
(C) filtering artefactual sites from the first and second compendium of reads, wherein the filtering comprises removing, from the first and second compendium of reads, recurring sites generated over a cohort of reference healthy samples; identifying shared CNVs/SVs between the first and second compendium as germ line mutations and removing said mutations from the first and second compendium of reads;
(D) detecting reads from a third subject-specific genome wide compendium of genetic markers in a third biological sample of the subject to generate a tumor-associated genome-wide representation of genetic markers in the third sample;
(E) normalizing each of the first, second and third compendium of reads to produce a first filtered read set for the first genome-wide compendium of reads, a second filtered read set for the second genome-wide compendium of reads, and a third filtered read set for the third genome-wide compendium of reads;
(F) computing an estimated tumor fraction (eTF) of the third biological samples, using the third filtered read set, by applying a background noise model to one or more integrative mathematical models, the one or more models producing a first eTF using the first filtered read set, and/or the one or more models producing a second eTF using the second filtered read set;
and
(G) detecting a residual disease in the subject if the estimated tumor fraction in the third biological sample exceeds an empirical threshold.

3. A system for detecting residual disease in a subject in need thereof,
comprising,
an analyzing unit, the analyzing unit comprising
a pre-filter engine configured and arranged to
receive a first subject-specific genome wide compendium of reads associated with genetic markers from a first biological sample of a subject, the first biological sample comprising a baseline sample and a normal sample, wherein the first compendium of reads each comprise reads of a single base pair length and wherein the baseline sample comprises a tumor sample or a plasma sample; and
filter artefactual sites from the first compendium of reads, wherein the filtering comprises removing, from the first compendium of genetic markers, recurring sites generated over a cohort of reference healthy samples, and/or identifying germ line mutations in peripheral blood mononuclear cells of the normal cell sample and removing said germ line mutations from the from the first compendium of genetic markers; and
a correction engine configured and arranged to
receive reads from a second subject-specific genome wide compendium of genetic markers in a second biological sample of the subject to generate a tumor-associated genome-wide representation of genetic markers in the second sample; and
filter noise from the first and second genome-wide compendium of reads using at least one error suppression protocol to produce a first filtered read set for the first genome-wide compendium of reads and a second filtered read set for the second genome-wide compendium of reads, wherein the at least one error suppression protocol comprises (a) calculating the probability that any single nucleotide variation in the first and second compendium is an artefactual mutation, and removing said mutation, wherein the probability is calculated as a function of features selected from the group consisting of mapping-quality (MQ), variant base-quality (MBQ), position-in-read (PIR), mean read base quality (MRBQ), and combinations thereof; and/or (b) removing artefactual mutations using discordance testing between independent replicates of the same DNA fragment generated from polymerase chain reaction or sequencing processing, and/or duplication consensus wherein artefactual mutations are identified and removed when lacking concordance across a majority of a given duplication family;
and
a computing unit configured and arranged to
compute an estimated tumor fraction (eTF) of the first and second biological sample using the first and second filtered read sets by applying a background noise model to one or more integrative mathematical models; and
detect a residual disease in the subject if the estimated tumor fraction in the second biological sample exceeds an empirical threshold.

4. A system for detecting residual disease in a subject in need thereof,
comprising,
a pre-filter engine configured and arranged to
receive a first subject-specific genome wide compendium of reads associated with genetic markers from a first biological sample of a subject, the first biological sample comprising a baseline sample, wherein the first compendium of reads each comprise reads of a single base pair length and wherein the baseline sample comprises a tumor sample or a plasma sample;
receive a second subject-specific genome wide compendium of reads associated with genetic markers from a second biological sample of a subject, the second biological sample comprising a peripheral blood mononuclear cell sample (PBMC), wherein the second compendium of genetic markers each comprise a copy number variation (CNV); and
filter artefactual sites from the first and second compendium of reads, wherein the filtering comprises removing, from the first and second compendium of reads, recurring sites generated over a cohort of reference healthy samples;
identifying shared CNVs between the first and second compendium as germ line mutations and removing said mutations from the first and second compendium of reads;
and
a correction engine configured and arranged to
receive reads from a third subject-specific genome wide compendium of genetic markers in a second biological sample of the subject to generate a tumor-associated genome-wide representation of genetic markers in the third sample; and
normalize each of the first, second and third compendium of reads to produce a first filtered read set for the first genome-wide compendium of reads, a second filtered read set for the second genome-wide compendium of reads, and a third filtered read set for the third genome-wide compendium of reads;
and
a computing unit configured and arranged to
compute an estimated tumor fraction (eTF) of the third biological samples, using the third filtered read set, by applying a background noise model to one or more integrative mathematical models, the one or more models producing a first eTF using the first filtered read set, and/or the one or more models producing a second eTF using the second filtered read set; and
detect a residual disease in the subject if the estimated tumor fraction in the third biological sample exceeds an empirical threshold.

5. (canceled)

6. The method of claim 1, wherein filtering recurring sites generated over a cohort of reference healthy samples comprises generating a panel of normal (PON) blacklist or mask.

7. The method of claim 1, wherein the normal sample comprises peripheral blood mononuclear cells (PBMC) and germ line mutations in PBMC that are removed in the artefactual site filtration step (B).

8. (canceled)

9. (canceled)

10. The method of claim 1, wherein step (D) comprises employing a machine learning (ML) algorithm, e.g., deep convolutional neural network (CNN), recurrent neural network (RNN), random forest (RF), support vector machine (SVM), discriminant analysis, nearest neighbor analysis (KNN), ensemble classifier, or a combination thereof; preferably, support vector machine (SVM), to filter artefactual noise.

11. The method of claim 1, wherein in step (D), the second error suppression step includes correction of artefactual mutations generated by PCR or sequencing using the comparison of independent replicates of the same original nucleic acid fragment.

12. The method of claim 11, wherein in step (D), the second error suppression step includes correction of artefactual mutations generated by paired-end 150 bp sequencing, resulting in overlapping paired reads (R1 and R2), and discordance between R1 and R2 pairs are corrected back to the corresponding reference genome.

13. The method of claim 1, wherein in step (D), the second error suppression step includes correction of duplication families generated during sequencing and/or PCR amplification, wherein the duplication families are recognized by 5′ and 3′ similarity as well as alignment position and wherein each duplication family is used to check the consensus of a specific mutation across independent replicates, thereby correcting artefactual mutations that do not show concordance in a majority of the duplication family.

14. The method of claim 1, wherein in step (E), the mathematical model integrates a relationship between the coverage, mutation load, number of detected mutations and the tumor fraction (TF).

15. The method of claim 1, wherein in step (E), the background noise calculation includes using patient specific mutation signature to calculate (1) the expected noise distribution over a cohort of healthy plasma samples (panel-of-normal or PON) or (2) the expected noise distribution across other patients (cross-patient analysis).

16. The method of claim 15, wherein the background noise model provides an estimated mean and standard-deviation (μ, σ) of artefactual mutation detection rate.

17. The method of claim 1 further comprising orthogonal integration of a secondary feature comprising fragment size shift.

18. The method of claim 17, wherein intra-patient fragment size shifts in the list of tumor-specific markers and random markers are analyzed using statistical methods, e.g., tests for significance or Gaussian mixture model (GMM).

19. (canceled)

20. The method of claim 2, wherein filtering recurring sites generated over a cohort of reference healthy samples comprises generating a panel of normal (PON) blacklist or mask.

21. The method of claim 2, wherein germ line events in PBMC are removed in the artefactual site filtration step (C).

22. (canceled)

23. (canceled)

24. The method of claim 2, wherein in step (C) comprises binning (to ≥500 bp windows) a region-of-interest (ROI) containing all the genomic segments of the somatic tumor CNV (sT_CNV) and somatic PBMC CNV (sP_CNV); estimating the depth coverage (read count) in each window from a follow-up plasma sample; and calculating median depth coverage per window.

25. (canceled)

26. The method of claim 2, wherein the normalization step includes normalizing depth coverage values to correct for GC-content and mappability biases by performing two LOESS regression curve-fitting on the bin-wise GC-fraction and mappability score.

27. The method of claim 2, wherein the normalization step includes batch-effect correction using a robust-zscore normalization, which is applied to each sample separately.

28. The method of claim 27, wherein the zscore normalization includes calculation of median and median-absolute-deviation (MAD) based on the neutral regions of each sample and normalizing all CNV bins are normalized by subtracting the median value and dividing the differential by MAD.

29. The method of claim 2, wherein step (E) includes calculating depth coverage skew and/or fragment size center-of-mass (COM) skew in the third sample in comparison to a panel of normal (PON) healthy plasma samples.

30. The method of claim 2, wherein step (E) includes calculation of tumor fraction by checking a linear dilution ratio between the cumulative signal detected at the follow-up plasma sample in comparison to the cumulative signal detected in the tumor sample.

31. The method of claim 2, wherein in step (F), the background noise model includes using patient specific CNV/SV signature to calculate (1) the expected noise distribution over a cohort of healthy plasma samples (panel-of-normal or PON) or (2) the expected noise distribution across other patients (cross-patient analysis).

32. The method of claim 31, wherein the background noise model provides an estimated mean and standard-deviation (μ, σ) of artefactual SNV/SV detection rate.

33. The method of claim 2, further comprising orthogonal integration of a secondary feature comprising fragment size shift.

34. The method of claim 33, wherein correlation between depth coverage skew and fragment size skew in CNV segments are analyzed to infer tumor fraction.
